SEQUENCE LISTING (69 sequences)

SEQ ID NO: 1 - protein, 450 aa, Artificial Sequence
  Synthetic
MRRLRFSPRSSFARTLLLIVTLLFASLVTTYLVVLNFAILPSLQQFNKVLAYEVRMLMTDKLQLEDGTQLVVPPAFRREIYRELGISLYSNEAAEEAGLRWAQHYEFLSHQMAQQLGGPTEVRVEVNKSSPVVWLKTWLSPNIWVRVPLTEIHQGDFSPLFRYTLAIMLLAIGGAWLFIRIQNRPLVDLEHAALQVGKGIIPPPLREYGASEVRSVTRAFNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVANMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 2 - protein, 450 aa, Artificial Sequence
  Synthetic
MRRLRFSPRSSFARTLLLIVTLLFASLVTTYLVVLNFAILPSLQQFNKVLAYEVRMLMTDKLQLEDGTQLVVPPAFRREIYRELGISLYSNEAAEEAGLRWAQHYEFLSHQMAQQLGGPTEVRVEVNKSSPVVWLKTWLSPNIWVRVPLTEIHQGDFSPLFRYTLAIMLLAIGGAWLFIRIQNRPLVDLEHAALQVGKGIIPPPLREYGASEVRSVTRAFNHMAAGVKQLADDRTLLMAGVSHALRTPLTRIRLATEMMSEQDGYLAESINKDIEECNAIIEQFIDYLRTGQEMPMEMADLNAVLGEVIAAESGYEREIETALYPGSIEVKMHPLSIKRAVANMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 3 - protein, 450 aa, Artificial Sequence
  Synthetic
MRRLRFSPRSSFARTLLLIVTLLFASLVTTYLVVLNFAILPSLQQFNKVLAYEVRMLMTDKLQLEDGTQLVVPPAFRREIYRELGISLYSNEAAEEAGLRWAQHYEFLSHQMAQQLGGPTEVRVEVNKSSPVVWLKTWLSPNIWVRVPLTEIHQGDFSPLFRYTLAIMLLAIGGAWLFIRIQNRPLVDLEHAALQVGKGIIPPPLREYGASEVRSVTRAFNHMAAGVKQLADDRTLLMAGVSHDLRAPLTRIRLATEMMSEQDGYLAESINKDIEECNAIIEQFIDYLRTGQEMPMEMADLNAVLGEVIAAESGYEREIETALYPGSIEVKMHPLSIKRAVANMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 4 - protein, 67 aa, Artificial Sequence
  Synthetic
MAAGVKQLADDRTLLMAGVSHDLRAPLTRIRLATEMMSEQDGYLAESINKDIEECNAIIEQFIDYLR

SEQ ID NO: 5 - protein, 303 aa, Artificial Sequence
  Synthetic
MAAGVKQLADDRTLLMAGVSHDLRTPLTRIRLATEMMSEQDGYLAESINKDIEECNAIIEQFIDYLRGGSIGGSIMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVANMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 6 - protein, 450 aa, Artificial Sequence
  Synthetic
MRRLRFSPRSSFARTLLLIVTLLFASLVTTYLVVLNFAILPSLQQFNKVLAYEVRMLMTDKLQLEDGTQLVVPPAFRREIYRELGISLYSNEAAEEAGLRWAQHYEFLSHQMAQQLGGPTEVRVEVNKSSPVVWLKTWLSPNIWVRVPLTEIHQGDFSPLFRYTLAIMLLAIGGAWLFIRIQNRPLVDLEHAALQVGKGIIPPPLREYGASEVRSVTRAFNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 7 - protein, 229 aa, Artificial Sequence
  Synthetic
HMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 8 - protein, 230 aa, Artificial Sequence
  Synthetic
NHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 9 - protein, 231 aa, Artificial Sequence
  Synthetic
FNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 10 - protein, 232 aa, Artificial Sequence
  Synthetic
AFNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 11 - protein, 233 aa, Artificial Sequence
  Synthetic
RAFNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 12 - protein, 234 aa, Artificial Sequence
  Synthetic
TRAFNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 13 - protein, 235 aa, Artificial Sequence
  Synthetic
VTRAFNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 14 - protein, 236 aa, Artificial Sequence
  Synthetic
SVTRAFNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 15 - protein, 237 aa, Artificial Sequence
  Synthetic
RSVTRAFNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 16 - protein, 238 aa, Artificial Sequence
  Synthetic
VRSVTRAFNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 17 - protein, 267 aa, Artificial Sequence
  Synthetic
MGSHMKQLEDKVEELLSKNYHLENEVARLVRSVTRAFNHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 18 - protein, 420 aa, Artificial Sequence
  Synthetic
ISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGGGGSGSHMKQLEDKVEELLSKNYHLENEVARLHMAAGVKQLADDRTLLMAGVSHDLRTPLTRIRLATEMMSEQDGYLAESINKDIEECNAIIEQFIDYLRTGQEMPMEMADLNAVLGEVIAAESGYEREIETALYPGSIEVKMHPLSIKRAVAKMVVNAARYGNGWIKVSSGTEPNRAWFQVEDDGPGIAPEQRKHLFQPFVRGDSARTISGTGLGLAIVQRIVDNHNGMLELGTSERGGLSIRAWLPVPVTRAQGTTKEG

SEQ ID NO: 19 - protein, 598 aa, Artificial Sequence
  Synthetic
MLKRCLSPLTLVNQVALIVLLSTAIGLAGMAVSGWLVQGVQGSAHAINKAGSLRMQSYRLLAAVPLSEKDKPLIKEMEQTAFSAELTRAAERDGQLAQLQGLQDYWRNELIPALMRAQNRETVSADVSQFVAGLDQLVSGFDRTTEMRIETVVLVHRVMAVFMALLLVFTIIWLRARLLQPWRQLLAMASAVSHRDFTQRANISGRNEMAMLGTALNNMSAELAESYAVLEQRVQEKTAGLEHKNQILSFLWQANRRLHSRAPLCERLSPVLNGLQNLTLLRDIELRVYDTDDEENHQEFTCQPDMTCDDKGCQLCPRGVLPVGDRGTTLKWRLADSHTQYGILLATLPQGRHLSHDQQQLVDTLVEQLTATLALDRHQERQQQLIVMEERATIARELHDSIAQSLSCMKMQVSCLQMQGDALPESSRELLSQIRNELNASWAQLRELLTTFRLQLTEPGLRPALEASCEEYSAKFGFPVKLDYQLPPRLVPSHQAIHLLQIAREALSNALKHSQASEVVVTVAQNDNQVKLTVQDNGCGVPENAIRSNHYGMIIMRDRAQSLRGDCRVRRRESGGTEVVVTFIPEKTFTDVQGDTHE

SEQ ID NO: 20 - protein, 349 aa, Artificial Sequence
  Synthetic
MATGTQPDAGQILNSLINSILLIDDNLAIHYANPAAQQLLAQSSRKLFGTPLPELLSYFSLNIELMQESLEAGQGFTDNEVTLVIDGRSHILSVTAQRMPDGMILLEMAPMDNQRRLSQEQLQHAQQVAARDLVRGLAHEIKNPLGGLRGAAQLLSKALPDPSLLEYTKVIIEQADRLRNLVDRLLGPQLPGTRVTESIHKVAERVVTLVSMELPDNVRLIRDYDPSLPELAHDPDQIEQVLLNIVRNALQALGPEGGEIILRTRTAFQLTLHGERYRLAARIDVEDNGPGIPPHLQDTLFYPMVSGREGGTGLGLSIARNLIDQHSGKIEFTSWPGHTEFSVYLPIRK

SEQ ID NO: 21 - protein, 449 aa, Artificial Sequence
  Synthetic
MKFTQRLSLRVRLTLIFLILASVTWLLSSFVAWKQTTDNVDELFDTQLMLFAKRLSTLDLNEINAADRMAQTPNRLKHGHVDDDALTFAIFTHDGRMVLNDGDNGEDIPYSYQREGFADGQLVGEDDPWRFVWMTSPDGKYRIVVGQEWEYREDMALAIVAGQLIPWLVALPIMLIIMMVLLGRELAPLNKLALALRMRDPDSEKPLNATGVPSEVRPLVESLNQLFARTHAMMVRERRFTSDAAHELRSPLTALKVQTEVAQLSDDDPQARKKALLQLHSGIDRATRLVDQLLTLSRLDSLDNLQDVAEIPLEDLLQSSVMDIYHTAQQAKIDVRLTLNAHSIKRTGQPLLLSLLVRNLLDNAVRYSPQGSVVDVTLNADNFIVRDNGPGVTPEALARIGERFYRPPGQTATGSGLGLSIVQRIAKLHGMNVEFGNAEQGGFEAKVSW

SEQ ID NO: 22 - protein, 363 aa, Artificial Sequence
  Synthetic
MHFLRRPISLRQRLILTIGAILLVFELISVFWLWHESTEQIQLFEQALRDNRNNDRHIMREIREAVASLIVPGVFMVSLTLFICYQAVRRITRPLAELQKELEARTADNLTPIAIHSATLEIEAVVSALNDLVSRLTSTLDNERLFTADVAHELRTPLAGVRLHLELLAKTHHIDVAPLVARLDQMMESVSQLLQLARAGQSFSSGNYQHVKLLEDVILPSYDELSTMLDQRQQTLLLPESAADITVQGDATLLRMLLRNLVENAHRYSPQGSNIMIKLQEDDGAVMAVEDEGPGIDESKCGELSKAFVRMDSRYGGIGLGLSIVSRITQLHHGQFFLQNRQETSGTRAWVRLKKDQYVANQI

SEQ ID NO: 23 - protein, 457 aa, Artificial Sequence
  Synthetic
MIGSLTARIFAIFWLTLALVLMLVLMLPKLDSRQMTELLDSEQRQGLMIEQHVEAELANDPPNDLMWWRRLFRAIDKWAPPGQRLLLVTTEGRVIGAERSEMQIIRNFIGQADNADHPQKKKYGRVELVGPFSVRDGEDNYQLYLIRPASSSQSDFINLLFDRPLLLLIVTMLVSTPLLLWLAWSLAKPARKLKNAADEVAQGNLRQHPELEAGPQEFLAAGASFNQMVTALERMMTSQQRLLSDISHELRTPLTRLQLGTALLRRRSGESKELERIETEAQRLDSMINDLLVMSRNQQKNALVSETIKANQLWSEVLDNAAFEAEQMGKSLTVNFPPGPWPLYGNPNALESALENIVRNALRYSHTKIEVGFAVDKDGITITVDDDGPGVSPEDREQIFRPFYRTDEARDRESGGTGLGLAIVETAIQQHRGWVKAEDSPLGGLRLVIWLPLYKRS

SEQ ID NO: 24 - protein, 452 aa, Artificial Sequence
  Synthetic
MKRLSITVRLTLLFILLLSVAGAGIVWTLYNGLASELKWRDDTTLINRTAQIKQLLIDGVNPDTLPVYFNRMMDVSQDILIIHGDSINKIVNRTNVSDGMLNNIPASETISAAGIYRSIINDTEIDALRINIDEVSPSLTVTVAKLASARHNMLEQYKINSIIICIVAIVLCSVLSPLLIRTGLREIKKLSGVTEALNYNDSREPVEVSALPRELKPLGQALNKMHHALVKDFERLSQFADDLAHELRTPINALLGQNQVTLSQTRSIAEYQKTIAGNIEELENISRLTENILFLARADKNNVLVKLDSLSLNKEVENLLDYLEYLSDEKEICFKVECNQQIFADKILLQRMLSNLIVNAIRYSPEKSRIHITSFLDTNSYLNIDIASPGTKINEPEKLFRRFWRGDNSRHSVGQGLGLSLVKAIAELHGGSATYHYLNKHNVFRITLPQRN

SEQ ID NO: 25 - protein, 486 aa, Artificial Sequence
  Synthetic
MKKLLRLFFPLSLRVRFLLATAAVVLVLSLAYGMVALIGYSVSFDKTTFRLLRGESNLFYTLAKWENNKLHVELPENIDKQSPTMTLIYDENGQLLWAQRDVPWLMKMIQPDWLKSNGFHEIEADVNDTSLLLSGDHSIQQQLQEVREDDDDAEMTHSVAVNVYPATSRMPKLTIVVVDTIPVELKSSYMVWSWFIYVLSANLLLVIPLLWVAAWWSLRPIEALAKEVRELEEHNRELLNPATTRELTSLVRNLNRLLKSERERYDKYRTTLTDLTHSLKTPLAVLQSTLRSLRSEKMSVSDAEPVMLEQISRISQQIGYYLHRASMRGGTLLSRELHPVAPLLDNLTSALNKVYQRKGVNISLDISPEISFVGEQNDFVEVMGNVLDNACKYCLEFVEISARQTDEHLYIVVEDDGPGIPLSKREVIFDRGQRVDTLRPGQGVGLAVAREITEQYEGKIVAGESMLGGARMEVIFGRQHSAPKDE

SEQ ID NO: 26 - protein, 480 aa, Artificial Sequence
  Synthetic
MVSKPFQRPFSLATRLTFFISLATIAAFFAFAWIMIHSVKVHFAEQDINDLKEISATLERVLNHPDETQARRLMTLEDIVSGYSNVLISLADSQGKTVYHSPGAPDIREFTRDAIPDKDAQGGEVYLLSGPTMMMPGHGHGHMEHSNWRMINLPVGPLVDGKPIYTLYIALSIDFHLHYINDLMNKLIMTASVISILIVFIVLLAVHKGHAPIRSVSRQIQNITSKDLDVRLDPQTVPIELEQLVLSFNHMIERIEDVFTRQSNFSADIAHEIRTPITNLITQTEIALSQSRSQKELEDVLYSNLEELTRMAKMVSDMLFLAQADNNQLIPEKKMLNLADEVGKVFDFFEALAEDRGVELRFVGDKCQVAGDPLMLRRALSNLLSNALRYTPTGETIVVRCQTVDHLVQVIVENPGTPIAPEHLPRLFDRFYRVDPSRQRKGEGSGIGLAIVKSIVVAHKGTVAVTSDARGTRFVITLPA

SEQ ID NO: 27 - protein, 16 aa, Artificial Sequence
  Synthetic
DFLVIYIEEAHASDGW

SEQ ID NO: 28 - protein, 12 aa, Artificial Sequence
  Synthetic
ADFLYIEAHDGW

SEQ ID NO: 29 - protein, 20 aa, Unknown
  HIV-1
QGPKEPFRDYVDRFYKTLRA

SEQ ID NO: 30 - protein, 25 aa, Artificial Sequence
  Synthetic
MKQLEDKVEELLSKNYHLENEVARL

SEQ ID NO: 31 - protein, 33 aa, Artificial Sequence
  Synthetic
GVTQLMREMLQLIKFQFSLNYQEESLSYQRLVT

SEQ ID NO: 32 - protein, 159 aa, Artificial Sequence
  Synthetic
MISLIAALAVDHVIGMENAMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 33 - protein, 245 aa, Artificial Sequence
  Synthetic
SLALSLTADQMVSALLDAEPPILYSEYDPTRPFSEASMMGLLTNLADRELVHMINWAKRVPGFVDLALHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSSKRMEHLYSMKCKNVVPLSDLLLEMLDAHRL

SEQ ID NO: 34 - protein, 108 aa, Artificial Sequence
  Synthetic
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 35 - protein, 15 aa, Artificial Sequence
  Synthetic
GGSGGSGGSGGSGGG

SEQ ID NO: 36 - protein, 8 aa, Artificial Sequence
  Synthetic
GGGSGGGS

SEQ ID NO: 37 - protein, 7 aa, Artificial Sequence
  Synthetic
GGGSGGG

SEQ ID NO: 38 - protein, 4 aa, Artificial Sequence
  Synthetic
GGGS

SEQ ID NO: 39 - protein, 4 aa, Artificial Sequence
  Synthetic
  MISC_FEATURE (1)..(4): may be repeated 1 - 5 times
GGGS

SEQ ID NO: 40 - protein, 4 aa, Artificial Sequence
  Synthetic
GSGG

SEQ ID NO: 41 - protein, 4 aa, Artificial Sequence
  Synthetic
  MISC_FEATURE (1)..(4): may be repeated 1 - 5 times
GSGG

SEQ ID NO: 42 - protein, 78 aa, Artificial Sequence
  Synthetic
APPTDVSLGDELHLDGEDVAMAHADALDDFDLDMLGDGDSPGPGFTPHDSAPYGALDMADFEFEQMFTDALGIDEYGG

SEQ ID NO: 43 - protein, 62 aa, Artificial Sequence
  Synthetic
EASGSGRADALDDFDLDMLGSDALDDFDLDMLGSDALDDFDLDMLGSDALDDFDLDMLINSR

SEQ ID NO: 44 - protein, 261 aa, Artificial Sequence
  Synthetic
SQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 45 - protein, 525 aa, Artificial Sequence
  Synthetic
RADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 46 - protein, 239 aa, Artificial Sequence
  Synthetic
MQENYKILVVDDDMRLRALLERYLTEQGFQVRSVANAEQMDRLLTRESFHLMVLDLMLPGEDGLSICRRLRSQSNPMPIIMVTAKGEEVDRIVGLEIGADDYIPKPFNPRELLARIRAVLRRQANELPGAPSQEEAVIAFGKFKLNLGTREMFREDEPMPLTSGEFAVLKALVSHPREPLSRDKLMNLARGREYSAMERSIDVQISRLRRMVEEDPAHPRYIQTVWGLGYVFVPDGSKA

SEQ ID NO: 47 - protein, 216 aa, Artificial Sequence
  Synthetic
MSNQEPATILLIDDHPMLRTGVKQLISMAPDITVVGEASNGEQGIELAESLDPDLILLDLNMPGMNGLETLDKLREKSLSGRIVVFSVSNHEEDVVTALKRGADGYLLKDMEPEDLLKALHQAAAGEMVLSEALTPVLAASLRANRATTERDVNQLTPRERDILKLIAQGLPNKMIARRLDITESTVKVHVKHMLKKMKLKSRVEAAVWVHQERIF

SEQ ID NO: 48 - protein, 469 aa, Artificial Sequence
  Synthetic
MQRGIVWVVDDDSSIRWVLERALAGAGLTCTTFENGAEVLEALASKTPDVLLSDIRMPGMDGLALLKQIKQRHPMLPVIIMTAHSDLDAAVSAYQQGAFDYLPKPFDIDEAVALVERAISHYQEQQQPRNVQLNGPTTDIIGEAPAMQDVFRIIGRLSRSSISVLINGESGTGKELVAHALHRHSPRAKAPFIALNMAAIPKDLIESELFGHEKGAFTGANTIRQGRFEQADGGTLFLDEIGDMPLDVQTRLLRVLADGQFYRVGGYAPVKVDVRIIAATHQNLEQRVQEGKFREDLFHRLNVIRVHLPPLRERREDIPRLARHFLQVAARELGVEAKLLHPETEAALTRLAWPGNVRQLENTCRWLTVMAAGQEVLIQDLPGELFESTVAESTSQMQPDSWATLLAQWADRALRSGHQNLLSEAQPELERTLLTTALRHTQGHKQEAARLLGWGRNTLTRKLKELGME

SEQ ID NO: 49 - protein, 229 aa, Artificial Sequence
  Synthetic
MARRILVVEDEAPIREMVCFVLEQNGFQPVEAEDYDSAVNQLNEPWPDLILLDWMLPGGSGIQFIKHLKRESMTRDIPVVMLTARGEEEDRVRGLETGADDYITKPFSPKELVARIKAVMRRISPMAVEEVIEMQGLSLDPTSHRVMAGEEPLEMGPTEFKLLHFFMTHPERVYSREQLLNHVWGTNVYVEDRTVDVHIRRLRKALEPGGHDRMVQTVRGTGYRFSTRF

SEQ ID NO: 50 - protein, 222 aa, Artificial Sequence
  Synthetic
MKILIVEDDTLLLQGLILAAQTEGYACDSVTTARMAEQSLEAGHYSLVVLDLGLPDEDGLHFLARIRQKKYTLPVLILTARDTLTDKIAGLDVGADDYLVKPFALEELHARIRALLRRHNNQGESELIVGNLTLNMGRRQVWMGGEELILTPKEYALLSRLMLKAGSPVHREILYNDIYNWDNEPSTNTLEVHIHNLRDKVGKARIRTVRGFGYMLVANEEN

SEQ ID NO: 51 - protein, 232 aa, Artificial Sequence
  Synthetic
MNKILLVDDDRELTSLLKELLEMEGFNVIVAHDGEQALDLLDDSIDLLLLDVMMPKKNGIDTLKALRQTHQTPVIMLTARGSELDRVLGLELGADDYLPKPFNDRELVARIRAILRRSHWSEQQQNNDNGSPTLEVDALVLNPGRQEASFDGQTLELTGTEFTLLYLLAQHLGQVVSREHLSQEVLGKRLTPFDRAIDMHISNLRRKLPDRKDGHPWFKTLRGRGYLMVSAS

SEQ ID NO: 52 - protein, 227 aa, Artificial Sequence
  Synthetic
MKLLIVEDEKKTGEYLTKGLTEAGFVVDLADNGLNGYHLAMTGDYDLIILDIMLPDVNGWDIVRMLRSANKGMPILLLTALGTIEHRVKGLELGADDYLVKPFAFAELLARVRTLLRRGAAVIIESQFQVADLMVDLVSRKVTRSGTRITLTSKEFTLLEFFLRHQGEVLPRSLIASQVWDMNFDSDTNAIDVAVKRLRGKIDNDFEPKLIQTVRGVGYMLEVPDGQ

SEQ ID NO: 53 - protein, 239 aa, Artificial Sequence
  Synthetic
MNTIVFVEDDAEVGSLIAAYLAKHDMQVTVEPRGDQAEETILRENPDLVLLDIMLPGKDGMTICRDLRAKWSGPIVLLTSLDSDMNHILALEMGACDYILKTTPPAVLLARLRLHLRQNEQATLTKGLQETSLTPYKALHFGTLTIDPINRVVTLANTEISLSTADFELLWELATHAGQIMDRDALLKNLRGVSYDGLDRSVDVAISRLRKKLLDNAAEPYRIKTVRNKGYLFAPHAWE

SEQ ID NO: 54 - protein, 223 aa, Artificial Sequence
  Synthetic
MKILLIEDNQRTQEWVTQGLSEAGYVIDAVSDGRDGLYLALKDDYALIILDIMLPGMDGWQILQTLRTAKQTPVICLTARDSVDDRVRGLDSGANDYLVKPFSFSELLARVRAQLRQHHTLNSTLEISGLRMDSVSQSVSRDNISITLTRKEFQLLWLLASRAGEIIPRTVIASEIWGINFDSDTNTVDVAIRRLRAKVDDPFPEKLIATIRGMGYSFVAVKK

SEQ ID NO: 55 - DNA, 21 bp, Artificial Sequence
  Synthetic
atttacattttgaaacatcta

SEQ ID NO: 56 - DNA, 16 bp, Artificial Sequence
  Synthetic
taccgctattgaggta

SEQ ID NO: 57 - DNA, 17 bp, Artificial Sequence
  Synthetic
tgcactaaaatggtgca

SEQ ID NO: 58 - DNA, 18 bp, Artificial Sequence
  Synthetic
  modified_base (9)..(9): n is A or T
  misc_feature (9)..(9): n is a, c, g, or t
  modified_base (11)..(11): n is A or T
  misc_feature (11)..(11): n is a, c, g, or t
  modified_base (18)..(18): n is C or T
  misc_feature (18)..(18): n is a, c, g, or t
ctgtcatananctgtcan

SEQ ID NO: 59 - DNA, 20 bp, Artificial Sequence
  Synthetic
tttgtattcagcccatatcg

SEQ ID NO: 60 - DNA, 21 bp, Artificial Sequence
  Synthetic
tttaattaaagacttcaagcg

SEQ ID NO: 61 - DNA, 20 bp, Artificial Sequence
  Synthetic
taattgtcaaatcagagtgc

SEQ ID NO: 62 - DNA, 20 bp, Artificial Sequence
  Synthetic
tttatgaggaatctctttgg

SEQ ID NO: 63 - DNA, 20 bp, Artificial Sequence
  Synthetic
ttcgaagtattccgcgtacg

SEQ ID NO: 64 - DNA, 22 bp, Artificial Sequence
  Synthetic
aacgatatgggctgaatacaaa

SEQ ID NO: 65 - DNA, 22 bp, Artificial Sequence
  Synthetic
ccgcttgaagtctttaattaaa

SEQ ID NO: 66 - DNA, 22 bp, Artificial Sequence
  Synthetic
aagcactctgatttgacaatta

SEQ ID NO: 67 - DNA, 22 bp, Artificial Sequence
  Synthetic
aaccaaagagattcctcataaa

SEQ ID NO: 68 - DNA, 22 bp, Artificial Sequence
  Synthetic
cacgtacgcggaatacttcgaa

SEQ ID NO: 69 - protein, 304 aa, Artificial Sequence
  Synthetic
MQENYKILVVDDDMRLRALLERYLTEQGFQVRSVANAEQMDRLLTRESFHLMVLDLMLPGEDGLSICRRLRSQSNPMPIIMVTAKGEEVDRIVGLEIGADDYIPKPFNPRELLARIRAVLRRQANELPGAPSQEEAVIAFGKFKLNLGTREMFREDEPMPLTSGEFAVLKALVSHPREPLSRDKLMNLARGREYSAMERSIDVQISRLRRMVEEDPAHPRYIQTVWGLGYVFVPDGSKAGSGEASGSGRADALDDFDLDMLGSDALDDFDLDMLGSDALDDFDLDMLGSDALDDFDLDMLINSR

(19) United States
(12) Patent Application Publication
(10) Pub. No.: US 2024/0018528 A1
(43) Pub. Date: Jan. 18, 2024

(54) A TUNABLE PHOSPHORYLATION-BASED FEEDBACK CONTROLLER OF MAMMALIAN GENE EXPRESSION

(71) Applicant: Massachusetts Institute of Technology, Cambridge, MA (US)

(72) Inventors: Ross D. Jones, Vancouver (CA); Yili Qian, Madison, WI (US); Domitilla Del Vecchio, Cambridge, MA (US); Ron Weiss, Newton, MA (US)

(73) Assignee: Massachusetts Institute of Technology, Cambridge, MA (US)

(21) Appl. No.: 18/039,583

(22) PCT Filed: Dec. 2, 2021
(86) PCT No.: PCT/US2021/061561

§ 371 Date: May 31, 2023

Related U.S. Application Data
(60) Provisional application No. 63/121,184, filed on Dec. 3, 2020.
(60) Provisional application No. 63/120,645, filed on Dec. 2, 2020.

(51) Int. Cl. C12N 15/63; C12N 9/12; A61K 48/00; A61K 38/45

(52) U.S. Cl. CPC C12N 15/635; C12N 9/12; C12Y 207/13003; A61K 48/0066; A61K 38/45; C12N 2830/001

(57) ABSTRACT

Provided herein are feedback controller circuits and cell state classifiers for tunable phosphorylation-based transcriptional regulation of gene expression in cells based on intracellular miRNA profiles and degradation by small molecules. Also provided are methods of using feedback controller circuits and cell state classifiers for determining the cell state, and methods of treating cells and subjects using feedback controller circuits and cell state classifiers encoding therapeutic output molecules.

### RELATED APPLICATIONS

This application claims the benefit under 35 U.S.C. § 119(e) of U.S. provisional Application No. 63/120,645 filed Dec. 2, 2020, and U.S. provisional Application No. 63/121,184 filed Dec. 3, 2020, each of which is incorporated by reference herein in its entirety.

### GOVERNMENT LICENSE RIGHTS

This invention was made with Government support under Grant No. FA9550-14-1-0060 awarded by the Air Force Office of Scientific Research, and under Grant No. MCB1840257 awarded by the National Science Foundation. The Government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Dec. 1, 2021, is named M065670519WO00-SEQ-NTJ, and is 120,125 bytes in size.

### BACKGROUND

Feedback control is a widespread engineering strategy for regulating processes such as vehicle speed, airplane altitude, and room temperature. Perturbations in the intracellular and extracellular environments, such as changes in the availability of a cell's resources, off-target gene regulation, and other changes in the cell state, can deleteriously affect gene expression in cells. Currently, intracellular feedback control for gene expression is not readily achievable in mammalian cells, and the few feedback controllers that do exist have limited tunability and do not provide high robustness to perturbations.

### SUMMARY

Provided herein are feedback controller circuits for expressing an output molecule under the control of an activable promoter that is activated by a phosphorylated exogenous activator, such that output molecule expression can be tuned by modulating the balance of kinase and phosphatase activity in the cell. Incorporating kinases, phosphatases, and response regulators of bacterial two-component signaling (TCS) systems, which do not occur in mammalian cells and are thus insulated from cross-talk by endogenous mammalian kinase and phosphatases, into feedback controller circuits allows output molecule expression to be tuned without interference from endogenous signaling pathways and without unduly affecting expression of endogenous mammalian genes. However, bacterial TCS systems include bifunctional histidine kinases with dual kinase and phosphatase activities, presenting a technical challenge to using such systems to tune gene expression to a desired level. To solve this problem, controller circuits of the present disclosure use separate kinase proteins and phosphatase regulators that are each derived from a bacterial TCS, such as the EnvZ-OmpR system, with the kinase protein favoring phosphorylation of the response regulator and the phosphatase regulator favoring dephosphorylation of the response regulator. Surprisingly, a strong phosphatase regulator was produced by (i) truncating an N-terminal portion of the wild-type sequence to remove the signaling domain and at least a portion of the HAMP domain; (ii) introducing a substitution corresponding to N343K of a wild-type EnvZ sequence to reduce ATP-binding, and thus kinase, activity; and (iii) assembling a dimer of two phosphatase monomers by fusing a dimerization domain to the truncated phosphatase domain, such that phosphatase monomers dimerized at an angle that would not interfere with phosphatase activity. This strong phosphatase regulator effectively dephosphorylated the response regulator, and therefore negatively regulated transcription of the output molecule, when incorporated into a feedback controller circuit. Furthermore, incorporation of a destabilization domain into the phosphatase regulator, which rendered the phosphatase regulator unstable unless a stabilizing small molecule was present, allowed the degree of negative regulation to be tuned by adding or withholding the small molecule.

Coupling expression of the output molecule and phosphatase regulator allowed expression of the output molecule, and thus phosphatase regulator abundance, to act as a negative feedback signal that decreased subsequent output molecule expression. This coupling imparted a robustness to the feedback controller circuit, such that expression of the output molecule was less sensitive to perturbations to cellular conditions. For example, off-target gene regulation of output molecule expression, such as suppression of translation by miRNAs or global up-regulation of other genes leading to sequestration of transcriptional resources, also reduced expression of the phosphatase regulator. Downregulation of phosphatase regulator expression shifted the kinase:phosphatase balance towards kinase activity, such that more activators were phosphorylated, allowing increased transcription of output molecule-encoding RNA to compensate for downregulation by perturbations mediated by other mechanisms. Therefore, feedback controllers of the present disclosure maintain a consistent level of output molecule expression in a cell, such that the output molecule's abundance is robust to changes in intracellular or extracellular conditions.

Accordingly, the present disclosure provides, in some aspects, a feedback controller circuit comprising:

(i) an input circuit comprising:
(a) a constitutive promoter operably linked to a nucleotide sequence encoding an activator; and
(b) a constitutive promoter operably linked to a nucleotide sequence encoding a kinase that phosphorylates the activator and produces a phosphorylated activator;

(ii) a tuning circuit comprising a promoter operably linked to a nucleotide sequence encoding a phosphatase regulator comprising a phosphatase domain and a degradation domain, wherein the phosphatase domain dephosphorylates the phosphorylated activator,

wherein the phosphatase domain comprises a mutation in a catalytic and ATP-binding domain and/or is truncated at its N-terminus relative to a phosphatase comprising the amino acid sequence of SEQ ID NO: 1; and

(iii) a signal circuit comprising an activatable promoter operably linked to a nucleotide sequence encoding an output molecule, wherein the activatable promoter is activatable by the phosphorylated activator. In some embodiments, the kinase, the phosphatase, and/or the activator are members of a bacterial two-component signaling system. In some embodiments, bacterial two-component system comprises a histidine kinase comprising an amino acid sequence motif of HEXXN, HEXXT, or HDXXXP, wherein X is any amino acid, and a response regulator.

In some embodiments, the phosphatase is a histidine kinase variant comprising an amino acid substitution in the E or D of the HEXXN, HEXXT, or HDXXXP motif. In some embodiments, the phosphatase comprises an alanine substitution in the E or D of the HEXXN, HEXXT, or HDXXXP motif. In some embodiments, the phosphatase comprises an amino acid substitution corresponding to a D244A substitution in SEQ ID NO: 1. In some embodiments, the phosphatase comprises the amino acid sequence of SEQ ID NO: 2. In some embodiments, the phosphatase comprises a dimerization and histidine phosphorylation (DHp) domain of EnvZ. In some embodiments, the phosphatase comprises the amino acid sequence of SEQ ID NO: 4.

In some embodiments, the phosphatase domain is a truncated EnvZ (EnvZt) phosphatase domain. In some embodiments, the phosphatase domain of the phosphatase regulator lacks a signaling domain and/or a HAMP domain of EnvZ. In some embodiments, the truncated EnvZ phosphatase domain comprises no more than 229, 230, 231, 232, 233, 234, 235, 236, 237, or 238 amino acids corresponding to the C-terminus of the amino acid sequence of SEQ ID NO: 6. In some embodiments, the phosphatase domain comprises a mutation in a catalytic and ATP-binding domain, wherein the binding affinity for ATP of a phosphatase comprising the mutation is at least 1.5, 2.0, 2.5, 3.0, 3.5, 4.0, 4.5, 5.0, 6.0, 7.0, 8.0, 9.0, or 10.0 times lower than binding affinity for ATP of a phosphatase comprising the amino acid sequence of SEQ ID NO: 1. In some embodiments, the phosphatase domain comprises a substitution at an amino acid corresponding to N343 of SEQ ID NO: 1. In some embodiments, the phosphatase domain comprises a lysine substitution at a position corresponding to N343 of SEQ ID NO: 1.

In some embodiments, the phosphatase regulator further comprises a leucine zipper domain that is covalently linked to the phosphatase domain. In some embodiments, the leucine zipper is a GCN4 domain. In some embodiments, the GCN4 domain comprises the amino acid sequence of SEQ ID NO: 30.

In some embodiments, the phosphatase regulator further comprises a degradation domain that is linked to the phosphatase domain or the leucine zipper domain. In some embodiments, the degradation domain is selected from the group consisting of PEST, DDd, DDe, and DDf. In some embodiments, the degradation domain is DDd.

In some embodiments, the phosphatase regulator comprises a plurality of monomers, wherein each monomer is a fusion protein comprising a phosphatase domain, a leucine zipper domain fused to the phosphatase domain, and one or more degradation domains fused to the phosphatase domain and/or leucine zipper domain. In some embodiments, each degradation domain is selected from the group consisting of PEST, DDd, DDe, and DDf. In some embodiments, each degradation domain is DDd. In some embodiments, the leucine zipper is a GCN4 domain. In some embodiments, the GCN4 domain comprises the amino acid sequence of SEQ ID NO: 30. In some embodiments, each monomer comprises an amino acid sequence with at least 80% sequence identity to the amino acid sequence of SEQ ID NO: 18. In some embodiments, each monomer comprises the amino acid sequence of SEQ ID NO: 18. In some embodiments, the phosphatase regulator comprises an amino acid sequence with at least 80% sequence identity to the amino acid sequence of SEQ ID NO: 18. In some embodiments, the phosphatase regulator comprises the amino acid sequence of SEQ ID NO: 18.

In some embodiments, the kinase is a variant of the histidine kinase an amino acid substitution in the N, T, or P of the HEXXN, HEXXT or HDXXXP motif. In some embodiments, the kinase comprises an alanine substitution in the N, T, or P of the HEXXN, HEXXT, or HDXXXP motif. In some embodiments, the histidine kinase is selected from the group consisting of: EnvZ, NarX, and PhoR. In some embodiments, the histidine kinase is an EnvZ or portion thereof. In some embodiments, the histidine kinase comprises the amino acid sequence of SEQ ID NO: 1. In some embodiments, the kinase comprises an amino acid substitution corresponding to a T247A substitution in SEQ ID NO: 1. In some embodiments, the kinase comprises the amino acid sequence of SEQ ID NO: 3. In some embodiments, the kinase comprises two DHp domains fused to a cytoplasmic domain of EnvZ. In some embodiments, the kinase comprises the amino acid sequence of SEQ ID NO: 5.

In some embodiments, the activator comprises a response regulator of the bacterial two-component system. In some embodiments, the activator comprises a response regulator of the bacterial two-component system fused to an activation domain. In some embodiments, the activation domain is selected from the group consisting of VP16, VP64, p65, and VPR. In some embodiments, the activation domain is VP64. In some embodiments, the response regulator is selected from the group consisting of OmpR, NarL, NtrC, and PhoB. In some embodiments, the response regulator is OmpR. In some embodiments, the activator comprises VP64 and OmpR. In some embodiments, the activator comprises an amino acid sequence with at least 80% sequence identity to the amino acid sequence of SEQ ID NO: 69. In some embodiments, the activatable promoter comprises one or more response elements that are capable of being bound by the activator. In some embodiments, the response element comprises one or more operators of the activator. In some embodiments, the activatable promoter further comprises a minimal promoter fused to the one or more response elements.

In some embodiments, the promoter of (ii) is the same as the activatable promoter of (iii). In some embodiments, the second sensor circuit and the signal circuit are comprised on the same nucleic acid, wherein the activatable promoter of (iii) controls transcription of the output molecule and the phosphatase regulator. In some embodiments, the promoter of (ii) is a constitutive promoter. In some embodiments, the tuning circuit and the signal circuit are comprised on different nucleic acids.

In some aspects, the present disclosure provides a cell state classifier comprising any of the feedback controller circuits provided herein, wherein the input circuit and the signal circuit each comprise one or more target sites for a first miRNA. In some embodiments, the one or more target sites for the first microRNA is located upstream and/or downstream of the nucleotide sequence encoding the activator and the nucleotide sequence encoding the kinase in the input circuit. In some embodiments, 4 target sites for the first microRNA are located upstream and/or downstream of the nucleotide sequence encoding the activator and the nucleotide sequence encoding the kinase in the input circuit. In some embodiments, the one or more target sites for the first microRNA is located upstream and/or downstream of the nucleotide sequence encoding the output molecule in the signal circuit. In some embodiments, 4 target sites for the first microRNA are located upstream and/or downstream of the nucleotide sequence encoding the output molecule in the signal circuit. In some embodiments, the tuning circuit comprises one or more target sequences for a second miRNA. In some embodiments, the one or more target sites for the second microRNA is located upstream and/or downstream of the nucleotide sequence encoding the phosphatase in the tuning circuit. In some embodiments, 4 target sites for the second microRNA are located upstream and/or downstream of the nucleotide sequence encoding the phosphatase in the tuning circuit.

In some embodiments of the feedback controller circuits or cell state classifiers provided herein, the output molecule is a detectable molecule. In some embodiments, the output molecule is a therapeutic molecule.

In some aspects, the present disclosure provides a cell comprising any of the feedback controller circuits or cell state classifiers provided herein. In some embodiments, the cell is a prokaryotic cell. In some embodiments, the cell is a bacterial cell. In some embodiments, the cell is a eukaryotic cell. In some embodiments, the eukaryotic cell is a plant cell, insect cell, fish cell, amphibian cell, reptilian cell, avian cell, or mammalian cell. In some embodiments, the mammalian cell is a human cell. In some embodiments, the cell is a diseased cell. In some embodiments, the cell is a cancer cell. In some embodiments, the cell does not express the first miRNA. In some embodiments, the cell expresses the second miRNA. In some embodiments, the cell expresses the first miRNA and the second miRNA. In some embodiments, the cell does not express the first miRNA and does not express the second miRNA.

In some aspects, the present disclosure provides a method comprising maintaining any of the cells provided herein. In some embodiments, the method further comprises detecting the output molecule. In some embodiments, the method further comprises classifying the cell based on expression of the output molecule.

In some aspects, the present disclosure provides a method comprising delivering any of the feedback controller circuits or cell state classifiers provided herein to a cell, and detecting the output molecule.

In some aspects, the present disclosure provides a method comprising delivering any of the feedback controller circuits or cell state classifiers provided herein to a cell, wherein the output molecule is a therapeutic molecule that is effective for treating the disease or disorder.

In some aspects, the present disclosure provides a method of determining the disease or disorder state of a cell, the method comprising delivering to the cell any of the feedback controller circuits or cell state classifiers provided herein. In some embodiments, the method further comprises detecting the output molecule. In some embodiments, detecting the output molecule in the cell indicates the disease or disorder. In some embodiments, lack of detection of the output molecule in the cell indicates the disease or disorder.

In some embodiments of the methods provided herein, the cell is a diseased cell. In some embodiments, the cell is a cancer cell.

In some aspects, the present disclosure provides a composition comprising any of the feedback controller circuits or cell state classifiers provided herein. In some aspects, the present disclosure provides a pharmaceutical composition comprising a pharmaceutically acceptable excipient, and a composition comprising any of the feedback controller circuits or cell state classifiers provided herein.

In some aspects, the present disclosure provides a method of treating a disease or disorder, the method comprising administering an effective amount of a composition provided herein to a subject in need thereof, wherein the output molecule is a therapeutic molecule that is effective for treating the disease or disorder.

In some aspects, the present disclosure provides a method of diagnosing a disease or disorder, the method comprising administering an effective amount of a composition provided herein to a subject in need thereof, and detecting the output molecule.

### BRIEF DESCRIPTION OF THE DRAWINGS

FIGS. 1A-1D illustrate a design of a phosphorylation-based feedback controller. FIG. 1A shows the futile cycle, which is a modular, composable basic building block of signaling pathways that can be exploited to build robust and tunable synthetic genetic circuits by manipulating the balance between a kinase and phosphatase acting on a transcription factor.

FIG. 1B illustrates the effects of phosphatase activity on the sensitivity of output molecule expression to kinase activity, such that output expression is less sensitive to kinase activity when phosphatase activities are higher. Brighter lines indicate higher levels of phosphatase activity. FIG. 1C illustrates how feedback control can make genetic circuits robust to environmental perturbations by using a sensor module, such as a phosphatase, to sense the output level and tune gene expression to bring actual output expression closer to a desired level. FIG. 1D shows how a closed loop (CL) genetic circuit, which responds to perturbations by modulating output expression, can render output expression more robust to perturbations than an open loop (OL) circuit that cannot respond to perturbations.

FIGS. 2A-2C illustrate bacterial two-component systems and their potential for use in phosphorylation-based regulation of gene expression. FIG. 2A illustrates signal transduction by two-component systems. Upon stimulation by an external signal, a transmembrane histidine kinase hydrolyzes ATP to phosphorylate a cytoplasmic response regulator. The response regulator is differentially active when phosphorylated, and so the external signal affects intracellular processes through downstream phosphorylation of the response regulator. FIG. 2B demonstrates the specificity of bacterial two-component systems. Histidine kinases are shown on the y-axis, and response regulators are shown on the x-axis. Square colors indicate the degree of luminescence observed in using two-component systems in which a given kinase was paired with a given response regulator, in cells containing an expression cassette with a promoter that was activatable by phosphorylated response regulator. Brighter squares indicate greater expression, and thus more efficient phosphorylation of the response regulator by the corresponding histidine kinase. FIG. 2C illustrates crosstalk between histidine kinases and response regulators in mammalian cells.

FIGS. 3A-3D illustrate design of kinases and phosphatases for use in a genetic circuit for tunable regulation of gene expression. FIG. 3A shows the bifunctional activity of many histidine kinases, which are capable of both phosphorylating and dephosphorylating a response regulator. Different domains of the histidine kinase EnvZ are shown, including the signaling domain, HAMP domain, dimerization and histidine phosphotransfer (DHp) domain, and catalytic and ATP-binding (CA) domain, are shown. Truncated variants lacking one or more domains, rearranged variants comprising desired combinations of one or more domains, and mutant variants with amino acid substitutions at catalytic sites, are shown. FIG. 3B shows the effects of truncations, rearrangements, and mutations on kinase and phosphatase activity in a genetic circuit that contains an OmpR-VP64 fusion protein as a response regulator, and an expression cassette that expresses an output molecule under the control of a promoter that is activated by phosphorylated OmpR. FIG. 3C shows the phosphatase activities of truncated and mutated EnvZ variants fused to a GCN4 leucine zipper domain to promote dimer formation. EnvZ-GCN4 fusion proteins lacked a signaling domain and some portion of the HAMP domain of EnvZ, and contained a T247A (m2) mutation, with some variants also containing an N343K (m3) mutation. FIG. 3D illustrates the effects of fusing one or more degradation domains (DDs) to the GCN4-EnvZ fusion proteins of FIG. 3C. DDd or DDe domains were fused to the N-terminus, C-terminus, or both N- and C-termini of GCN4-EnvZ fusion proteins, and the effects of adding a corresponding small molecule (DDd=TMP; DDe=4-OHT) to stabilize the degradation domain, were measured.

FIGS. 4A-4E show the design of a tunable input-output controller and sensitivity of the controller to tuning by different methods. FIG. 4A shows the design of a controller circuit, containing a constitutively active EnvZm2 kinase and GCN4-EnvZt 10m3 phosphatase, which together regulate phosphorylation of an OmpR-VP64 response regulator, with pOmpR-VP64 activating transcription of an output molecule. FIG. 4B shows the biochemical mechanisms by which inputs are converted into outputs, and the responsiveness of output expression to the ratio of kinase:phosphatase. FIG. 4C shows the responsiveness of the controller when both kinase and phosphatase activities are tuned by modulating DNA dosage. FIG. 4D shows the responsiveness of the controller when kinase activity is tuned by modulating DNA dosage, and phosphatase activity is tuned by including an miRNA target site on the input circuit encoding the GCN4-EnvZt10m3 fusion protein. FIG. 4E shows the responsiveness of the controller when DDd is fused to the N-terminus of the GCN4-EnvZt10m3 fusion protein, and increasing amounts of TMP are added to stabilize the phosphatase.

FIGS. 5A-5G show the implementation of a feedback controller to modulate a cell's responsiveness to changes in gene expression, such as through translational repression or indirect transcriptional repression. FIG. 5A shows a feedback controller in which a constitutively active kinase EnvZm2 phosphorylates an OmpR-VP64 response regulator, with pOmpR-VP64 driving expression of both an output molecule and a DDd-GCN4-EnvZt10m3 phosphatase regulator, which can dephosphorylate the pOmpR-VP64 response regulator, thereby downregulating output expression. Perturbations causing changes in gene expression are mediated by a) expressing a miRNA targeting a cognate miRNA target site on the mRNA encoding the output molecule and phosphatase regulator; and/or b) expressing Gal4-VPR, a potent transcriptional activator that sequesters a cell's transcriptional resources, indirectly inhibiting transcription of signal circuit mRNA via the squelching effect. FIG. 5B shows responsiveness to changes in kinase activity of OL circuits that are not regulated by a phosphatase, compared to responsiveness of the CL circuit shown in FIG. 5A. FIG. 5C shows the responsiveness of an OL circuit, the CL circuit of FIG. 5A when cells were incubated with increasing concentrations of TMP to stabilize the phosphatase regulator, and a control CL circuit lacking a degradation domain, in which the phosphatase regulator was stable even the absence of TMP. FIG. 5D shows the effects of increasing kinase activity and TMP concentration on the ability of the feedback controller of FIG. 5A to regulate output expression. FIG. 5E shows the variability, in terms of interquartile range, of output expression levels of CL circuits and a control OL circuit (Fluc2), across levels of kinase activity. FIG. 5F shows the interquartile ranges of output expression levels of OL circuits and a control CL circuit, across levels of kinase activity. FIG. 5G shows the interquartile ranges of CL circuits containing DDd-GCN4-EnvZt10m3 phosphatase regulators as a function of TMP concentration and kinase activity. FIG. 5H shows the interquartile ranges of FIGS. 5E-5G as functions of output expression.

FIGS. 6A-6C show the robustness and responsiveness of the feedback controller circuit shown in FIG. 5A to perturbations that affect translation of the phosphatase, such as changes in the abundance of miRNA. FIG. 6A shows the responsiveness of an OL circuit (left panel), a CL circuit expressing EnvZm3t10 in the presence of TMP (middle panel), and a CL circuit expressing EnvZm3t10 in the absence of TMP (right panel), as functions of the abundance of miR-FF4, an miRNA that prevents translation of the output molecule and phosphatase regulator. FIG. 6B shows the fold-change in output molecule expression as a function of kinase activity. FIG. 6C shows the robustness of each of the circuits tested in FIGS. 5A-5G at a given level of output.

FIGS. 7A-7C show the robustness and responsiveness of the feedback controller circuit shown in FIG. 5A to perturbations that affect transcription of the output molecule, such as upregulation of other genes sequestering transcriptional resources. FIG. 7A shows the responsiveness of an OL circuit (left panel), a CL circuit expressing EnvZm3t10 in the presence of TMP (middle panel), and a CL circuit expressing EnvZm3t10 in the absence of TMP (right panel), as functions of the abundance of Gal4-VPR, which potently activates many other genes, sequestering transcriptional resources and limiting expression of the output molecule and phosphatase regulator. FIG. 7B shows the fold-change in output molecule expression as a function of kinase activity. FIG. 7C shows the robustness of each of the circuits tested in FIGS. 5A-5G at a given level of output.

### DETAILED DESCRIPTION

Provided herein are feedback controller circuits for expressing an output molecule under the control of an activable promoter that is activated by a phosphorylated exogenous activator, such that output molecule expression can be tuned by modulating the balance of kinase and phosphatase activity in the cell. Incorporating kinases, phosphatases, and response regulators of bacterial two-component signaling (TCS) systems, which do not occur in mammalian cells and are thus insulated from cross-talk by endogenous mammalian kinase and phosphatases, into feedback controller circuits allows output molecule expression to be tuned without interference from endogenous signaling pathways and without unduly affecting expression of endogenous mammalian genes. However, bacterial TCS systems include bifunctional histidine kinases with dual kinase and phosphatase activities, presenting a technical challenge to using such systems to tune gene expression to a desired level. To solve this problem, controller circuits of the present disclosure use separate kinase proteins and phosphatase regulators that are each derived from a bacterial TCS, such as the EnvZ-OmpR system, with the kinase protein favoring phosphorylation of the response regulator and the phosphatase regulator favoring dephosphorylation of the response regulator. Surprisingly, a strong phosphatase regulator was produced by (i) truncating an N-terminal portion of the wild-type sequence to remove the signaling domain and at least a portion of the HAMP domain; (ii) introducing a substitution corresponding to N343K of a wild-type EnvZ sequence to reduce ATP-binding, and thus kinase, activity; and (iii) assembling a dimer of two phosphatase monomers by fusing a dimerization domain to the truncated phosphatase domain, such that phosphatase monomers dimerized at an angle that would not interfere with phosphatase activity. This strong phosphatase regulator effectively dephosphorylated the response regulator, and therefore negatively regulated transcription of the output molecule, when incorporated into a feedback controller circuit. Furthermore, incorporation of a destabilization domain into the phosphatase regulator, which rendered the phosphatase regulator unstable unless a stabilizing small molecule was present, allowed the degree of negative regulation to be tuned by adding or withholding the small molecule.

Coupling expression of the output molecule and phosphatase regulator allowed expression of the output molecule, and thus phosphatase regulator abundance, to act as a negative feedback signal that decreased subsequent output molecule expression. This coupling imparted a robustness to the feedback controller circuit, such that expression of the output molecule was less sensitive to perturbations to cellular conditions. For example, off-target gene regulation of output molecule expression, such as suppression of translation by miRNAs or global up-regulation of other genes leading to sequestration of transcriptional resources, also reduced expression of the phosphatase regulator. Downregulation of phosphatase regulator expression shifted the kinase:phosphatase balance towards kinase activity, such that more activators were phosphorylated, allowing increased transcription of output molecule-encoding RNA to compensate for downregulation by perturbations mediated by other mechanisms. Therefore, feedback controllers of the present disclosure maintain a consistent level of output molecule expression in a cell, such that the output molecule's abundance is robust to changes in intracellular or extracellular conditions.

### Phosphorylation-Based Feedback Controller Circuits

Aspects of the present disclosure relate to feedback controller circuits that express an output molecule under the control of an activatable promoter, which is activated by the phosphorylated form of an activator, such that transcription of mRNA encoding the output molecule depends on the presence of phosphorylated activator. Phosphorylation of the activator by the kinase produces a phosphorylated activator. A “kinase” is an enzyme that catalyzes the transfer of a phosphate group from ATP to a specified molecule (e.g., a protein), and the process is known as “phosphorylation.” During phosphorylation, the substrate (e.g., a protein) gains a phosphate group and the high-energy ATP molecule donates a phosphate group, producing a phosphorylated substrate and ADP. Kinases are part of the larger family of phosphotransferases. The phosphorylation state of a molecule, e.g., protein, lipid, or carbohydrate, can affect its activity, reactivity, and its ability to bind other molecules. Therefore, kinases are critical in metabolism, cell signaling, protein regulation, cellular transport, secretory processes, and many other cellular pathways. For kinases that phosphorylate proteins, the phosphate group may be transferred to a serine, a threonine, tyrosine, or a histidine residue in the protein. Non-limiting examples of kinases include serine kinases, threonine kinases, tyrosine kinase, and histidine kinases.

Feedback controller circuits of the present disclosure comprise:

(i) an input circuit comprising:
(a) a constitutive promoter operably linked to a nucleotide sequence encoding an activator; and
(b) a constitutive promoter operably linked to a nucleotide sequence encoding a kinase that phosphorylates the activator and produces a phosphorylated activator;

(ii) a tuning circuit comprising a promoter operably linked to a nucleotide sequence encoding a phosphatase regulator comprising a phosphatase domain and a degradation domain, wherein the phosphatase domain dephosphorylates the phosphorylated activator, wherein the phosphatase domain comprises a mutation in a catalytic and ATP-binding domain and/or is truncated at its N-terminus relative to a phosphatase comprising the amino acid sequence of SEQ ID NO: 1; and

(iii) a signal circuit comprising an activatable promoter operably linked to a nucleotide sequence encoding an output molecule, wherein the activatable promoter is activatable by the phosphorylated activator. The kinase of the input circuit phosphorylates the activator, producing a phosphorylated activator, which causes a conformational change in the activator, allowing it to bind to its target DNA sequence, e.g., a promoter. Thus, the extent of transcription, and thus output molecule expression, may be regulated by modulating the balance of phosphorylated and dephosphorylated forms of the activator in a cell. For example, expression of the kinase may be constitutive, while expression of the phosphatase regulator is sensitive to one or more intracellular (e.g., miRNA abundance) or extracellular (e.g., nutrient availability, receptor stimulation) conditions, such that the extent of phosphatase activity may fluctuate with changing conditions, and therefore tune the degree of output molecule expression in response to such changes. In another example, expression of the phosphatase is tightly linked to output molecule expression, such that expression of the output molecule is part of a negative feedback loop where activation of the activatable promoter encoding the output molecule and phosphatase regulator indirectly reduces subsequent output molecule expression. The strength of this feedback may be modulated by altering the stability of the phosphatase regulator, either inherently by altering the phosphatase amino acid sequence, or transiently by adding or withholding a stabilizing small molecule that stabilizes a degradation domain on the phosphatase regulator.

In some embodiments, the nucleotide sequences encoding kinase and the activator is placed under the control of one constitutive promoter. As such, the activator and the kinase is transcribed into one polycistronic mRNA containing two different coding sequences (or open reading frames (ORF)). In some embodiments, the activator and the kinase are translated from the polycistronic mRNA into a fusion protein, provided that the kinase is able to phosphorylate the activator when it is fused to the activator. Alternatively, in some embodiments, translation of the activator and the kinase can initiate and proceed independently on the two coding sequences, producing the activator and the kinase as individual proteins, e.g., by placing an internal ribosomal entry site (IRES) between the nucleotide sequence encoding the activator and the nucleotide sequence encoding the kinase. In some embodiments, the nucleotide sequence encoding the activator and the nucleotide sequence encoding the kinase are each placed under control of a constitutive promoter, and is transcribed and translated independently into individual proteins. In some embodiments, the two constitutive promoters are the same promoter. In other embodiments, the two constitutive promoters are different promoters.

In some embodiments, the kinase, the phosphatase, and/or the activator are members of a bacterial two-component system. A “bacterial two-component system” is a stimulus-response coupling mechanism that allows bacterial cells to sense and respond to changes in many different environmental conditions. Two-component systems typically consist of a membrane-bound histidine kinase (HK) that senses a specific environmental stimulus and a corresponding response regulator (RR) that mediates the cellular response, mostly transcriptional regulation (e.g., activation or repression) of target genes. The histidine kinase and its cognate RR typically functions orthogonally, and are referred to hear in as a “HK—RR pair.” In some embodiments, the kinase, the phosphatase, and the activator of the feedback controller circuit described herein are derived from the same HK—RR pair.

Without wishing to be bound by scientific theory, each HK—RR pair accomplishes signal transduction through the phosphorylation of the response regulator by the histidine kinase. Histidine kinases are typically homodimeric transmembrane proteins that contain a dimerization and histidine phosphorylation domain (DHp).

A “response regulator (RR)” is a protein that mediates a cell's response to changes in its environment as part of a two-component regulatory system. Response regulators are coupled to specific histidine kinases which serve as sensors of environmental changes. Many response regulators are transcriptional factors, and their binding to DNA is controlled by this conformational change. Response regulators typically consist of a receiver domain and one or more effector domains, although in some cases they possess only a receiver domain and exert their effects through protein-protein interactions.

In the absence of signal input, HKs act as a phosphatase on their cognate RR. Upon extracellular signal induction, the HK will auto-phosphorylate a conserved histidine residue in the dimerization and histidine phosphorylation (DHp) domain of itself. The phosphate group is then rapidly transferred to the HK's cognate RR protein on a conserved aspartate residue in the receiver domain of the protein. This phosphate group causes a conformational change in the RR that allows it to bind a target DNA sequence (e.g., a promoter) and activate/repress the expression of a gene.

A large number of bacterial two-component systems (e.g., HK and RR pairs) are known and may be used in accordance with the present disclosure. Information regarding bacterial two-component systems are available in the art, e.g., in public databases such as p2cs.org. Non-limiting examples of E. coli two-component systems include the EnvZ-OmpR system, the NarX-NarL system, the NtrB-NtrC system, and the PhoR-PhoB system.

In some embodiments, the histidine kinase in the bacterial two-components system comprises a conserved amino acid sequence motif of HEXXN, HEXXT, or HDXXXP, wherein X is any amino acid, such as any naturally occurring amino acid. In some embodiments, the bacterial two-component system comprises a response regulator and a histidine kinase comprising a conserved amino acid sequence motif of HEXXN, HEXXT, or HDXXXP, wherein X is any amino acid, such as any naturally occurring amino acid. The histidine (H) in the conserved motif can undergo autophosphorylation upon a signal input and became a phosphohistidine (H p). Non-limiting examples of histidine kinases from bacterial two-component systems that may be used in accordance with the present disclosure include: Osmolarity sensor protein (EnvZ), Nitrate/nitrite sensor protein (NarX), Nitrogen regulation protein NR(II) (NtrB), Phosphate regulon sensor protein PhoR (PhoR), BaeS, BasS, CpxA, CusS, KdpD, PhoQ, QseC, RstB, and YedV. In some embodiments, the histidine kinase is selected from the group consisting of BasS, CpxA, YedV, NarX, QseC, PhoQ, CusS, and EnvZ. In some embodiments, the histidine kinase is EnvZ, which comprises a conserved HEXXT motif. The histidine kinases may be from any bacterial species that genetically encodes them, e.g., Escherichia coli. Gene and protein sequences of the histidine kinases described herein are available in the art, e.g., in public databases such as the GENBANK®. Non-limiting examples of histidine kinases and their amino acid sequences are given in Table 1.

TABLE 1
Exemplary histidine kinases and phosphatases

| Histidine | |
|---|---|
| kinase | Amino Acid Sequence |
| EnvZ | MRRLRFSPRSSFARTLLLIVILLFASLVTTYLVVLNFAILPSLQQFNKVLAYEVRMLMTDKLQLE |
| | DGTQLVVPPAFRREIYRELGISLYSNEAAEEAGLRWAQHYEFLSHQMAQQLGGPTEVRVEVNKSS |
| | PVVWLKTWLSPNIWVRVPLTEIHQGDFSPLFRYTLAIMLLAIGGAWLFIRIQNRPLVDLEHAALQ |
| | VGKGIIPPPLREYGASEVRSVTRAFNHMAAGVKQLADDRILLMAGVSHDLRTPLTRIRLATEMMS |
| | EQDGYLAESINKDIEECNAIIEQFIDYLRTGQEMPMEMADLNAVLGEVIAAESGYEREIETALYP |
| | GSIEVKMHPLSIKRAVANMVVNAARYGNGWIKVSSGTEPNRAWFQVEDDGPGIAPEQRKHLFQPF |
| | VRGDSARTISGTGLGLAIVQRIVDNHNGMLELGTSERGGLSIRAWLPVPVTRAQGTTKEG (SEQ |
| | ID NO: 1) |
| EnvZ D244A | MRRLRFSPRSSFARTLLLIVILLFASLVTTYLVVLNFAILPSLQQFNKVLAYEVRMLMTDKLQLE |
| (Phosphatase) | DGTQLVVPPAFRREIYRELGISLYSNEAAEEAGLRWAQHYEFLSHQMAQQLGGPTEVRVEVNKSS |
| | PVVWLKTWLSPNIWVRVPLTEIHQGDFSPLFRYTLAIMLLAIGGAWLFIRIQNRPLVDLEHAALQ |
| | VGKGIIPPPLREYGASEVRSVTRAFNHMAAGVKQLADDRILLMAGVSHALRTPLTRIRLATEMMS |
| | EQDGYLAESINKDIEECNAIIEQFIDYLRTGQEMPMEMADLNAVLGEVIAAESGYEREIETALYP |
| | GSIEVKMHPLSIKRAVANMVVNAARYGNGWIKVSSGTEPNRAWFQVEDDGPGIAPEQRKHLFQPF |
| | VRGDSARTISGTGLGLAIVQRIVDNHNGMLELGTSERGGLSIRAWLPVPVTRAQGTTKEG (SEQ |
| | ID NO: 2) |
| EnvZ T247A | MRRLRFSPRSSFARTLLLIVILLFASLVTTYLVVLNFAILPSLQQFNKVLAYEVRMLMTDKLQLE |
| (Kinase) | DGTQLVVPPAFRREIYRELGISLYSNEAAEEAGLRWAQHYEFLSHQMAQQLGGPTEVRVEVNKSS |
| | PVVWLKTWLSPNIWVRVPLTEIHQGDFSPLFRYTLAIMLLAIGGAWLFIRIQNRPLVDLEHAALQ |
| | VGKGIIPPPLREYGASEVRSVTRAFNHMAAGVKQLADDRILLMAGVSHDLRAPLTRIRLATEMMS |
| | EQDGYLAESINKDIEECNAIIEQFIDYLRTGQEMPMEMADLNAVLGEVIAAESGYEREIETALYP |
| | GSIEVKMHPLSIKRAVANMVVNAARYGNGWIKVSSGTEPNRAWFQVEDDGPGIAPEQRKHLFQPF |
| | VRGDSARTISGTGLGLAIVQRIVDNHNGMLELGTSERGGLSIRAWLPVPVTRAQGTTKEG (SEQ |
| | ID NO: 3) |
| EnvZ DHp | MAAGVKQLADDRILLMAGVSHDLRAPLTRIRLATEMMSEQDGYLAESINKDIEECNAIIEQFIDY |
| (Phosphatase) | LR (SEQ ID NO: 4) |
| EnvZ DHp- | MAAGVKQLADDRILLMAGVSHDLRTPLTRIRLATEMMSEQDGYLAESINKDIEECNAIIEQFIDY |
| DHp-CA | LRGGSIGGSIMAAGVKQLADDRILLMAGVSHDLRTPLTRIRLATEMMSEQDGYLAESINKDIEEC |
| (Kinase) | NAIIEQFIDYLRTGQEMPMEMADLNAVLGEVIAAESGYEREIETALYPGSIEVKMHPLSIKRAVA |
| | NMVVNAARYGNGWIKVSSGTEPNRAWFQVEDDGPGIAPEQRKHLFQPFVRGDSARTISGTGLGLA |
| | IVQRIVDNHNGMLELGTSERGGLSIRAWLPVPVTRAQGTTKEG (SEQ ID NO: 5) |
| EnvZ N343K | MRRLRFSPRSSFARTLLLIVTLLFASLVTTYLVVLNFAILPSLQQFNKVLAYEVRMLMTDKLQLE |
| (EnvZm3 | DGTQLVVPPAFRREIYRELGISLYSNEAAEEAGLRWAQHYEFLSHQMAQQLGGPTEVRVEVNKSS |
| phosphatase) | PVVWLKTWLSPNIWVRVPLTEIHQGDFSPLFRYTLAIMLLAIGGAWLFIRIQNRPLVDLEHAALQ |
| | VGKGIIPPPLREYGASEVRSVTRAFNHMAAGVKQLADDRILLMAGVSHDLRTPLTRIRLATEMMS |
| | EQDGYLAESINKDIEECNAIIEQFIDYLRTGQEMPMEMADLNAVLGEVIAAESGYEREIETALYP |
| | GSIEVKMHPLSIKRAVAKMVVNAARYGNGWIKVSSGTEPNRAWFQVEDDGPGIAPEQRKHLFQPF |
| | VRGDSARTISGTGLGLAIVQRIVDNHNGMLELGTSERGGLSIRAWLPVPVTRAQGTTKEG (SEQ |
| | ID NO: 6) |
| Truncated | HMAAGVKQLADDRTLLMAGVSHDLRTPLTRIRLATEMMSEQDGYLAESINKDIEECNAIIEQFID |
| EnvZ N343K | YLRTGQEMPMEMADLNAVLGEVIAAESGYEREIETALYPGSIEVKMHPLSIKRAVAKMVVNAARY |
| (EnvZm3t1 | GNGWIKVSSGTEPNRAWFQVEDDGPGIAPEQRKHLFQPFVRGDSARTISGTGLGLAIVQRIVDNH |
| phosphatase) | NGMLELGTSERGGLSIRAWLPVPVTRAQGTTKEG (SEQ ID NO: 7) |
| Truncated | NHMAAGVKQLADDRILLMAGVSHDLRTPLTRIRLATEMMSEQDGYLAESINKDIEECNAIIEQFI |
| EnvZ N343K | DYLRTGQEMPMEMADLNAVLGEVIAAESGYEREIETALYPGSIEVKMHPLSIKRAVAKMVVNAAR |
| (EnvZm3t2 | YGNGWIKVSSGTEPNRAWFQVEDDGPGIAPEQRKHLFQPFVRGDSARTISGTGLGLAIVQRIVDN |
| phosphatase) | HNGMLELGTSERGGLSIRAWLPVPVTRAQGTTKEG (SEQ ID NO: 8) |
| Truncated | FNHMAAGVKQLADDRTLLMAGVSHDLRTPLTRIRLATEMMSEQDGYLAESINKDIEECNAIIEQF |
| EnvZ N343K | IDYLRTGQEMPMEMADLNAVLGEVIAAESGYEREIETALYPGSIEVKMHPLSIKRAVAKMVVNAA |
| (EnvZm3t3 | RYGNGWIKVSSGTEPNRAWFQVEDDGPGIAPEQRKHLFQPFVRGDSARTISGTGLGLAIVQRIVD |
| phosphatase) | NHNGMLELGTSERGGLSIRAWLPVPVTRAQGTTKEG (SEQ ID NO: 9) |
| Truncated | AFNHMAAGVKQLADDRTLLMAGVSHDLRTPLTRIRLATEMMSEQDGYLAESINKDIEECNAIIEQ |
| EnvZ N343K | FIDYLRTGQEMPMEMADLNAVLGEVIAAESGYEREIETALYPGSIEVKMHPLSIKRAVAKMVVNA |
| (EnvZm3t4 | ARYGNGWIKVSSGTEPNRAWFQVEDDGPGIAPEQRKHLFQPFVRGDSARTISGTGLGLAIVQRIV |
| phosphatase) | DNHNGMLELGTSERGGLSIRAWLPVPVTRAQGTTKEG (SEQ ID NO: 10) |
| Truncated | RAFNHMAAGVKQLADDRILLMAGVSHDLRTPLTRIRLATEMMSEQDGYLAESINKDIEECNAIIE |
| EnvZ N343K | QFIDYLRTGQEMPMEMADLNAVLGEVIAAESGYEREIETALYPGSIEVKMHPLSIKRAVAKMVVN |
| (EnvZm3t5 | AARYGNGWIKVSSGTEPNRAWFQVEDDGPGIAPEQRKHLFQPFVRGDSARTISGTGLGLAIVQRI |
| phosphatase) | VDNHNGMLELGTSERGGLSIRAWLPVPVTRAQGTTKEG (SEQ ID NO: 11) |
| Truncated | TRAFNHMAAGVKQLADDRILLMAGVSHDLRTPLTRIRLATEMMSEQDGYLAESINKDIEECNAII |
| EnvZ N343K | EQFIDYLRTGQEMPMEMADLNAVLGEVIAAESGYEREIETALYPGSIEVKMHPLSIKRAVAKMVV |
| (EnvZm3t6 | NAARYGNGWIKVSSGTEPNRAWFQVEDDGPGIAPEQRKHLFQPFVRGDSARTISGTGLGLAIVQR |
| phosphatase) | IVDNHNGMLELGTSERGGLSIRAWLPVPVTRAQGTTKEG (SEQ ID NO: 12) |
| Truncated | VTRAFNHMAAGVKQLADDRILLMAGVSHDLRTPLTRIRLATEMMSEQDGYLAESINKDIEECNAI |
| EnvZ N343K | IEQFIDYLRTGQEMPMEMADLNAVLGEVIAAESGYEREIETALYPGSIEVKMHPLSIKRAVAKMV |
| (EnvZm3t7 | VNAARYGNGWIKVSSGTEPNRAWFQVEDDGPGIAPEQRKHLFQPFVRGDSARTISGTGLGLAIVQ |
| phosphatase) | RIVDNHNGMLELGTSERGGLSIRAWLPVPVTRAQGTTKEG (SEQ ID NO: 13) |
| Truncated | SVTRAFNHMAAGVKQLADDRTLLMAGVSHDLRTPLTRIRLATEMMSEQDGYLAESINKDIEECNA |
| EnvZ N343K | IIEQFIDYLRTGQEMPMEMADLNAVLGEVIAAESGYEREIETALYPGSIEVKMHPLSIKRAVAKM |
| (EnvZm3t8 | VVNAARYGNGWIKVSSGTEPNRAWFQVEDDGPGIAPEQRKHLFQPFVRGDSARTISGTGLGLAIV |
| phosphatase) | QRIVDNHNGMLELGTSERGGLSIRAWLPVPVTRAQGTTKEG (SEQ ID NO: 14) |
| Truncated | RSVTRAFNHMAAGVKQLADDRILLMAGVSHDLRTPLTRIRLATEMMSEQDGYLAESINKDIEECN |
| EnvZ N343K | AIIEQFIDYLRTGQEMPMEMADLNAVLGEVIAAESGYEREIETALYPGSIEVKMHPLSIKRAVAK |
| (EnvZm3t9 | MVVNAARYGNGWIKVSSGTEPNRAWFQVEDDGPGIAPEQRKHLFQPFVRGDSARTISGTGLGLAI |
| phosphatase) | VQRIVDNHNGMLELGTSERGGLSIRAWLPVPVTRAQGTTKEG (SEQ ID NO: 15) |
| Truncated | VRSVTRAFNHMAAGVKQLADDRTLLMAGVSHDLRTPLTRIRLATEMMSEQDGYLAESINKDIEEC |
| EnvZ N343K | NAIIEQFIDYLRTGQEMPMEMADLNAVLGEVIAAESGYEREIETALYPGSIEVKMHPLSIKRAVA |
| (EnvZm3t10 | KMVVNAARYGNGWIKVSSGTEPNRAWFQVEDDGPGIAPEQRKHLFQPFVRGDSARTISGTGLGLA |
| phosphatase) | IVQRIVDNHNGMLELGTSERGGLSIRAWLPVPVTRAQGTTKEG (SEQ ID NO: 16) |
| Truncated | MGSHMKQLEDKVEELLSKNYHLENEVARLVRSVTRAFNHMAAGVKQLADDRILLMAGVSHDLRTP |
| EnvZ N343K | LTRIRLATEMMSEQDGYLAESINKDIEECNAIIEQFIDYLRTGQEMPMEMADLNAVLGEVIAAES |
| (EnvZm3t10 | GYEREIETALYPGSIEVKMHPLSIKRAVAKMVVNAARYGNGWIKVSSGTEPNRAWFQVEDDGPGI |
| phosphatase) | APEQRKHLFQPFVRGDSARTISGTGLGLAIVQRIVDNHNGMLELGTSERGGLSIRAWLPVPVTRA |
| with GCN4 | QGTTKEG (SEQ ID NO: 17) |
| domain | |
| Truncated | MISLIAALAVDYVIGMENAMPWNLPADLAWFKRNTLNKPVIMGRHTWESIGRPLPGRKNIILSSQ |
| EnvZ N343K | PSTDDRVTWVKSVDEAIAACGDVPEIMVIGGGRVIEQFLPKAQKLYLTHIDAEVEGDTHFPDYEP |
| (EnvZm3t10 | DDWESVFSEFHDADAQNSHSYCFEILERRGGGGSGSHMKQLEDKVEELLSKNYHLENEVARLHMA |
| phosphatase) | AGVKQLADDRTLLMAGVSHDLRTPLTRIRLATEMMSEQDGYLAESINKDIEECNAIIEQFIDYLR |
| with GCN4 | TGQEMPMEMADLNAVLGEVIAAESGYEREIETALYPGSIEVKMHPLSIKRAVAKMVVNAARYGNG |
| leucine zipper | WIKVSSGTEPNRAWFQVEDDGPGIAPEQRKHLFQPFVRGDSARTISGTGLGLAIVQRIVDNHNGM |
| domain and | LELGTSERGGLSIRAWLPVPVTRAQGTTKEG (SEQ ID NO: 18) |
| DDd | |
| degradation | |
| domain | |
| NarX | MLKRCLSPLTLVNQVALIVLLSTAIGLAGMAVSGWLVQGVQGSAHAINKAGSLRMQSYRLLAAVP |
| | LSEKDKPLIKEMEQTAFSAELTRAAERDGQLAQLQGLQDYWRNELIPALMRAQNRETVSADVSQF |
| | VAGLDQLVSGFDRTTEMRIETVVLVHRVMAVFMALLLVFTIIWLRARLLQPWRQLLAMASAVSHR |
| | DFTQRANISGRNEMAMLGTALNNMSAELAESYAVLEQRVQEKTAGLEHKNQILSFLWQANRRLHS |
| | RAPLCERLSPVLNGLQNLTLLRDIELRVYDTDDEENHQEFTCQPDMTCDDKGCQLCPRGVLPVGD |
| | RGTTLKWRLADSHTQYGILLATLPQGRHLSHDQQQLVDTLVEQLTATLALDRHQERQQQLIVMEE |
| | RATIARELHDSIAQSLSCMKMQVSCLQMQGDALPESSRELLSQIRNELNASWAQLRELLTTFRLQ |
| | LTEPGLRPALEASCEEYSAKFGFPVKLDYQLPPRLVPSHQAIHLLQIAREALSNALKHSQASEVV |
| | VTVAQNDNQVKLTVQDNGCGVPENAIRSNHYGMIIMRDRAQSLRGDCRVRRRESGGTEVVVTFIP |
| | EKTFTDVQGDTHE (SEQ ID NO: 19) |
| NtrB | MATGTQPDAGQILNSLINSILLIDDNLAIHYANPAAQQLLAQSSRKLFGTPLPELLSYFSLNIEL |
| | MQESLEAGQGFTDNEVTLVIDGRSHILSVTAQRMPDGMILLEMAPMDNQRRLSQEQLQHAQQVAA |
| | RDLVRGLAHEIKNPLGGLRGAAQLLSKALPDPSLLEYTKVIIEQADRLRNLVDRLLGPQLPGTRV |
| | TESIHKVAERVVTLVSMELPDNVRLIRDYDPSLPELAHDPDQIEQVLLNIVRNALQALGPEGGEI |
| | ILRTRTAFQLTLHGERYRLAARIDVEDNGPGIPPHLQDTLFYPMVSGREGGTGLGLSIARNLIDQ |
| | HSGKIEFTSWPGHTEFSVYLPIRK (SEQ ID NO: 20) |
| QseC | MKFTQRLSLRVRLTLIFLILASVTWLLSSFVAWKQTTDNVDELFDTQLMLFAKRLSTLDLNEINA |
| | ADRMAQTPNRLKHGHVDDDALTFAIFTHDGRMVLNDGDNGEDIPYSYQREGFADGQLVGEDDPWR |
| | FVWMTSPDGKYRIVVGQEWEYREDMALAIVAGQLIPWLVALPIMLIIMMVLLGRELAPLNKLALA |
| | LRMRDPDSEKPLNATGVPSEVRPLVESLNQLFARTHAMMVRERRFTSDAAHELRSPLTALKVQTE |
| | VAQLSDDDPQARKKALLQLHSGIDRATRLVDQLLILSRLDSLDNLQDVAEIPLEDLLQSSVMDIY |
| | HTAQQAKIDVRLILNAHSIKRIGQPLLLSLLVRNLLDNAVRYSPQGSVVDVILNADNFIVRDNGP |
| | GVTPEALARIGERFYRPPGQTATGSGLGLSIVQRIAKLHGMNVEFGNAEQGGFEAKVSW (SEQ |
| | ID NO: 21) |
| BasS | MHFLRRPISLRQRLILTIGAILLVFELISVFWLWHESTEQIQLFEQALRDNRNNDRHIMREIREA |
| | VASLIVPGVFMVSLTLFICYQAVRRITRPLAELQKELEARTADNLTPIAIHSATLEIEAVVSALN |
| | DLVSRLISTLDNERLFTADVAHELRTPLAGVRLHLELLAKTHHIDVAPLVARLDQMMESVSQLLQ |
| | LARAGQSFSSGNYQHVKLLEDVILPSYDELSTMLDQRQQTLLLPESAADITVQGDATLLRMLLRN |
| | LVENAHRYSPQGSNIMIKLQEDDGAVMAVEDEGPGIDESKCGELSKAFVRMDSRYGGIGLGLSIV |
| | SRITQLHHGQFFLQNRQETSGTRAWVRLKKDQYVANQI (SEQ ID NO: 22) |
| CpxA | MIGSLTARIFAIFWLTLALVLMLVLMLPKLDSRQMTELLDSEQRQGLMIEQHVEAELANDPPNDL |
| | MWWRRLFRAIDKWAPPGQRLLLVTTEGRVIGAERSEMQIIRNFIGQADNADHPQKKKYGRVELVG |
| | PFSVRDGEDNYQLYLIRPASSSQSDFINLLFDRPLLLLIVTMLVSTPLLLWLAWSLAKPARKLKN |
| | AADEVAQGNLRQHPELEAGPQEFLAAGASFNQMVTALERMMTSQQRLLSDISHELRTPLTRLQLG |
| | TALLRRRSGESKELERIETEAQRLDSMINDLLVMSRNQQKNALVSETIKANQLWSEVLDNAAFEA |
| | EQMGKSLTVNFPPGPWPLYGNPNALESALENIVRNALRYSHTKIEVGFAVDKDGITITVDDDGPG |
| | VSPEDREQIFRPFYRTDEARDRESGGTGLGLAIVETAIQQHRGWVKAEDSPLGGLRLVIWLPLYK |
| | RS (SEQ ID NO: 23) |
| YedV | MKRLSITVRLILLFILLLSVAGAGIVWTLYNGLASELKWRDDTTLINRTAQIKQLLIDGVNPDTL |
| | PVYFNRMMDVSQDILIIHGDSINKIVNRINVSDGMLNNIPASETISAAGIYRSIINDTEIDALRI |
| | NIDEVSPSLTVTVAKLASARHNMLEQYKINSIIICIVAIVLCSVLSPLLIRTGLREIKKLSGVTE |
| | ALNYNDSREPVEVSALPRELKPLGQALNKMHHALVKDFERLSQFADDLAHELRTPINALLGQNQV |
| | TLSQTRSIAEYQKTIAGNIEELENISRLTENILFLARADKNNVLVKLDSLSLNKEVENLLDYLEY |
| | LSDEKEICFKVECNQQIFADKILLQRMLSNLIVNAIRYSPEKSRIHITSFLDINSYLNIDIASPG |
| | TKINEPEKLFRRFWRGDNSRHSVGQGLGLSLVKAIAELHGGSATYHYLNKHNVFRITLPQRN |
| | (SEQ ID NO: 24) |
| PhoQ | MKKLLRLFFPLSLRVRFLLATAAVVLVLSLAYGMVALIGYSVSFDKTTFRLLRGESNLFYTLAKW |
| | ENNKLHVELPENIDKQSPTMTLIYDENGQLLWAQRDVPWLMKMIQPDWLKSNGFHEIEADVNDTS |
| | LLLSGDHSIQQQLQEVREDDDDAEMTHSVAVNVYPATSRMPKLTIVVVDTIPVELKSSYMVWSWF |
| | IYVLSANLLLVIPLLWVAAWWSLRPIEALAKEVRELEEHNRELLNPATTRELTSLVRNLNRLLKS |
| | ERERYDKYRITLIDLTHSLKTPLAVLQSTLRSLRSEKMSVSDAEPVMLEQISRISQQIGYYLHRA |
| | SMRGGTLLSRELHPVAPLLDNLISALNKVYQRKGVNISLDISPEISFVGEQNDFVEVMGNVLDNA |
| | CKYCLEFVEISARQTDEHLYIVVEDDGPGIPLSKREVIFDRGQRVDTLRPGQGVGLAVAREITEQ |
| | YEGKIVAGESMLGGARMEVIFGRQHSAPKDE (SEQ ID NO: 25) |
| CusS | MVSKPFQRPFSLATRLTFFISLATIAAFFAFAWIMIHSVKVHFAEQDINDLKEISATLERVLNHP |
| | DETQARRLMTLEDIVSGYSNVLISLADSQGKTVYHSPGAPDIREFTRDAIPDKDAQGGEVYLLSG |
| | PTMMMPGHGHGHMEHSNWRMINLPVGPLVDGKPIYTLYIALSIDFHLHYINDLMNKLIMTASVIS |
| | ILIVFIVLLAVHKGHAPIRSVSRQIQNITSKDLDVRLDPQTVPIELEQLVLSFNHMIERIEDVFT |
| | RQSNFSADIAHEIRTPITNLITQTEIALSQSRSQKELEDVLYSNLEELTRMAKMVSDMLFLAQAD |
| | NNQLIPEKKMLNLADEVGKVFDFFEALAEDRGVELRFVGDKCQVAGDPLMLRRALSNLLSNALRY |
| | TPTGETIVVRCQTVDHLVQVIVENPGTPIAPEHLPRLFDRFYRVDPSRQRKGEGSGIGLAIVKSI |
| | VVAHKGTVAVTSDARGTRFVITLPA (SEQ ID NO: 26) |

In some embodiments, the kinase encoded by the input and/or tuning circuits of the feedback controller circuit is a variant of the histidine kinase in the bacterial two-component system. As demonstrated herein, the kinase and phosphatase activities of a histidine kinase from a bacterial two-component system are separated by modifying its amino acid sequence. Typically, to generate a kinase, the asparagine (N), threonine (T), or proline (P) of the conserved motif is substituted by a different amino acid, e.g., replaced by alanine (A). To generate a phosphatase, the glutamic acid (E) or aspartic acid (D) of the conserved motif is substituted by a different amino acid, e.g., replaced by A.

Accordingly, in some embodiments, the kinase of the input circuit comprises an amino acid substitution in N, T, or P of the HEXXN, HEXXT, or HDXXXP motifs, respectively. In some embodiments, the kinase of the input circuit comprises an alanine (A) substitution in the N, T, or P of the HEXXN, HEXXT, or HDXXXP motif, respectively. As such, in some embodiments, the kinase of the input circuit comprises a motif of the amino acid sequence of: HEXXA or HDXXXA, wherein X is any amino acid, such as any naturally occurring amino acid.

In some embodiments, the phosphatase of the tuning circuit comprises an amino acid substitution in E or D of the HEXXN, HEXXT, or HDXXXP motif, respectively. In some embodiments, the phosphatase of the tuning circuit comprises an alanine substitution in E or D position of the HEXXN, HEXXT, or HDXXXP motif, respectively. As such, in some embodiments, the phosphatase of the tuning circuit comprises a motif of the amino acid sequence of HAXXN, HAXXT, or HAXXXP, wherein X is any amino acid, such as any naturally occurring amino acid.

In some embodiments, the histidine kinase is EnvZ (e.g., E. coli EnvZ, SEQ ID NO: 1) and the kinase and phosphatase of the feedback controller circuit described herein are variants of EnvZ. In some embodiments, the kinase encoded by the input circuit is an EnvZ variant comprising an amino acid substitution corresponding to a T247A substitution in SEQ ID NO: 1. In some embodiments, the kinase encoded by the input circuit comprises the amino acid sequence of SEQ ID NO: 3. In some embodiments, the kinase encoded by the input circuit comprises an amino acid sequence that is at least 80%, at least 85%, at least 90%, at least 95%, or at least 99% identical to the amino acid sequence of SEQ ID NO: 3, and comprises an amino acid substitution corresponding to a T247A substitution in SEQ ID NO: 1. In some embodiments, the kinase encoded by the input circuit comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 3, and comprises an amino acid substitution corresponding to a T247A substitution in SEQ ID NO: 1. In some embodiments, the kinase encoded by the input circuit comprises an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 3, and comprises an amino acid substitution corresponding to a T247A substitution in SEQ ID NO: 1. In some embodiments, the kinase encoded by the input circuit consists of the amino acid sequence of SEQ ID NO: 3.

In some embodiments, the kinase encoded by the input circuit comprises two DHp domains of EnvZ fused to a cytoplasmic domain of EnvZ. In some embodiments, the kinase encoded by the input circuit comprises the amino acid sequence of SEQ ID NO: 5. In some embodiments, the kinase encoded by the input circuit comprises an amino acid sequence that is at least 80%, at least 85%, at least 90%, at least 95%, or at least 99% identical to the amino acid sequence of SEQ ID NO: 5. In some embodiments, the kinase encoded by the input circuit may comprise an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 5. In some embodiments, the kinase encoded by the input circuit may comprise an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 5. In some embodiments, the kinase of the input circuit consists of the amino acid sequence of SEQ ID NO: 5.

In some embodiments, the phosphatase encoded by the tuning circuit is an EnvZ variant comprising an amino acid substitution corresponding to a D244A substitution in SEQ ID NO: 1. In some embodiments, the phosphatase encoded by the tuning circuit comprises the amino acid sequence of SEQ ID NO: 2. In some embodiments, the phosphatase encoded by the tuning circuit comprises an amino acid sequence that is at least 80%, at least 85%, at least 90%, at least 95%, or at least 99% identical to the amino acid sequence of SEQ ID NO: 2, and comprises an amino acid substitution corresponding to a D244A substitution in SEQ ID NO: 1. In some embodiments, the phosphatase encoded by the tuning circuit comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 2, and comprises an amino acid substitution corresponding to a D244A substitution in SEQ ID NO: 1. In some embodiments, the phosphatase encoded by the tuning circuit comprises an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 2, and comprises an amino acid substitution corresponding to a D244A substitution in SEQ ID NO: 1. In some embodiments, the phosphatase encoded by the tuning circuit consists of the amino acid sequence of SEQ ID NO: 2.

In some embodiments, the phosphatase encoded by the tuning circuit comprises a dimerization and histidine phosphorylation (DHp) domain of EnvZ. In some embodiments, the phosphatase encoded by the tuning circuit comprises the amino acid sequence of SEQ ID NO: 4. In some embodiments, the phosphatase encoded by the tuning circuit comprises an amino acid sequence that is at least 80%, at least 85%, at least 90%, at least 95%, or at least 99% identical to the amino acid sequence of SEQ ID NO: 4. In some embodiments, the phosphatase encoded by the tuning circuit comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 4. In some embodiments, the phosphatase encoded by the tuning circuit comprises an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 4. In some embodiments, the phosphatase of the tuning circuit consists of the amino acid sequence of SEQ ID NO: 4.

In some embodiments, the phosphatase encoded by the tuning circuit comprises a truncated form of a phosphatase of any of the bacterial two-component systems provided herein. A truncated form of a protein (e.g., phosphatase) refers to a portion of the protein comprising fewer amino acids than the full-length amino acid sequence of the protein. For example, if a first protein consists of an amino acid sequence that is 500 amino acids long, a second protein consisting of amino acids 1-400 of the first protein is said to be a truncated form of the first protein. Specifically, a second protein lacking amino acids from the beginning of the amino acid sequence of a first protein is said to comprise an N-terminal truncation relative to the first protein or be truncated at the N-terminus, while a second protein lacking amino acids from the end of the amino acid sequence is said to comprise a C-terminal truncation relative to the first protein or be truncated at its C-terminus. In some embodiments, the phosphatase comprises a truncated EnvZ (EnvZt) phosphatase domain. In some embodiments, the EnvZt phosphatase domain lacks a signaling domain and/or a HAMP domain of EnvZ. In some embodiments, the EnvZt phosphatase domain comprises a portion of the HAMP domain of EnvZ, but does not comprise a full-length HAMP domain or a signaling domain of EnvZ. In some embodiments, the EnvZt phosphatase domain comprises no more than 229, 230, 231, 232, 233, 234, 235, 236, 237, or 238 amino acids corresponding to the C-terminus of the amino acid sequence of SEQ ID NO: 6. In some embodiments, the EnvZt phosphatase domain comprises 238 amino acids corresponding to the C-terminus of the amino acid sequence of SEQ ID NO: 6. In some embodiments, the EnvZt phosphatase domain comprises 237 amino acids corresponding to the C-terminus of the amino acid sequence of SEQ ID NO: 6. In some embodiments, the EnvZt phosphatase domain comprises 236 amino acids corresponding to the C-terminus of the amino acid sequence of SEQ ID NO: 6. In some embodiments, the EnvZt phosphatase domain comprises 235 amino acids corresponding to the C-terminus of the amino acid sequence of SEQ ID NO: 6. In some embodiments, the EnvZt phosphatase domain comprises 234 amino acids corresponding to the C-terminus of the amino acid sequence of SEQ ID NO: 6. In some embodiments, the EnvZt phosphatase domain comprises 233 amino acids corresponding to the C-terminus of the amino acid sequence of SEQ ID NO: 6. In some embodiments, the EnvZt phosphatase domain comprises 232 amino acids corresponding to the C-terminus of the amino acid sequence of SEQ ID NO: 6. In some embodiments, the EnvZt phosphatase domain comprises 231 amino acids corresponding to the C-terminus of the amino acid sequence of SEQ ID NO: 6. In some embodiments, the EnvZt phosphatase domain comprises 230 amino acids corresponding to the C-terminus of the amino acid sequence of SEQ ID NO: 6. In some embodiments, the EnvZt phosphatase domain comprises 229 amino acids corresponding to the C-terminus of the amino acid sequence of SEQ ID NO: 6.

In some embodiments, the phosphatase encoded by the tuning circuit comprises a mutation in a catalytic and ATP-binding (CA) domain. In some embodiments, the mutation in the CA domain of the phosphatase reduces the phosphatase's binding affinity for ATP by at least 1.5 times, 2.0 times, 2.5 times, 3.0 times, 3.5 times, 4.0 times, 4.5 times, 5.0 times, 6.0 times, 7.0 times, 8.0 times, 9.0 times, or 10.0 times compared to a wild-type phosphatase that does not have the mutation. In some embodiments, the mutation in the CA domain of the phosphatase reduces the phosphatase's binding affinity for ATP by about 1.5 times, 2.0 times, 2.5 times, 3.0 times, 3.5 times, 4.0 times, 4.5 times, 5.0 times, 6.0 times, 7.0 times, 8.0 times, 9.0 times, or 10.0 times compared to a wild-type phosphatase that does not have the mutation. Mutations in the CA domain of a phosphatase may be insertions, deletions, or and/or substitutions. In some embodiments, the mutation is a substitution. In some embodiments, the mutation is a substitution of an amino acid corresponding to an asparagine (N) at position 343 of the amino acid sequence of SEQ ID NO: 1. In some embodiments, the mutation is a substitution of a lysine (K) for an amino acid corresponding to an asparagine (N) at position 343 of the amino acid sequence of SEQ ID NO: 1. In some embodiments, the phosphatase comprises a substitution corresponding to an N343K substitution in the amino acid sequence of SEQ ID NO: 1.

In some embodiments, the phosphatase regulator of the feedback controller circuits provided herein comprises a multimerization domain. Multimerization domains promote the association of proteins having the same or compatible multimerization domains, such that expression of individual subunits leads to the self-assembly of multimeric structures through interactions between the multimerization domains. Previous attempts to generate a strong phosphatase that could effectively impart negative feedback into the feedback controller circuits of the present disclosure were unsuccessful, until the phosphatase domains were fused to a GCN4 domain to generate a fusion protein capable of self-assembling into a multimer comprising multiple phosphatase domains, such as through GCN4 dimerization. Thus, fusing a multimerization domain to the phosphatase domain markedly improved the tunability and effectiveness of feedback in the feedback controller circuits provided herein in a manner that was not reasonably predictable by one of ordinary skill in the art.

Any suitable peptide domain capable of promoting self-assembly/multimerization can be used. Non-limiting examples of multimerization domains known to those of skill in the art and suitable for use in feedback controller circuits of the present disclosure include: helices containing at least one helix or a structure formed by a helix, a coil and another helix; coiled coil structures; dimerization domains found in cell surface signaling receptors, antibody Fc or hinge regions, leucine zippers, STAT protein N-terminal domains, FK506-binding protein (FKBP), LexA C-terminal domains, nuclear receptors, FkpA N-terminal domains, orange carotenoid protein of A. maxima, M1 protein of influenza A virus, neuraminidase (NA) protein of influenza A virus, and fuculose aldolase of E. coli. See, e.g., O'Shea, Science. 254: 539 (1991); Barahmand-Pour et al. Curr Top Microbiol Immunol. 211: 121-128 (1996); Klemm et al. Annu Rev Immunol. 16: 569-592 (1998); Klemm et al. Annu Rev Immunol. 16: 569-592 (1998); Ho et al. Nature. 382: 822-826 (1996); and Pomeranz et al. Biochem. 37: 965 (1998). Other examples include amino acids 325-410 of bovine papillomavirus E2 protein; Type 1 deiodinase motifs having the sequence DFLVIYIEEAHASDGW (SEQ ID NO: 27) or ADFLYIEAHDGW (SEQ ID NO: 28); HIV-1 Capsid protein motif having the sequence QGPKEPFRDYVDRFYKTLRA (SEQ ID NO: 29); leucine zipper dimerization of yeast GCN4 motif having the sequence MKQLEDKVEELLSKNYHLENEVARL (SEQ ID NO: 30); leucine zipper dimerization domain of E. coli transcriptional antiterminator protein; and BglG motif having the sequence GVTQLMREMLQLIKFQFSLNYQEESLSYQRLVT (SEQ ID NO: 31).

In some embodiments, the phosphatase regulator comprises a leucine zipper domain that is linked to the phosphatase domain. In some embodiments, the leucine zipper domain is a GCN4 domain. In some embodiments, the GCN4 domain comprises an amino acid sequence with at least 80%, at least 85%, at least 90%, at least 95%, at least 97%, at least 98%, at least 99%, or up to 100% sequence identity to the amino acid sequence of SEQ ID NO: 30. In some embodiments, the leucine zipper domain comprises the amino acid sequence of SEQ ID NO: 30.

In some embodiments, the phosphatase regulator of the feedback controller circuits provided herein comprises a degradation domain that is linked to the phosphatase domain and/or the leucine zipper domain of the phosphatase regulator. A destabilization domain (DD) is an amino acid sequence that is readily identified and degraded by one or more components of protein quality control machinery within cells of a particular organism, such as, but not limited to, the ubiquitin proteosome system of eukaryotes. A destabilization domain may also be referred to as a “degron” or “degradation domain”. A destabilization domain may be a complete protein or a subset of a protein (e.g., an amino acid sequence corresponding to one or more domains within a protein, or part of a domain thereof). In such embodiments, fusion with a destabilization domain causes the one or more fused proteins to be targeted by degradation machinery within cells, thereby decreasing their intracellular quantity.

In some embodiments, the destabilization domain is a destabilization domain derived from the dihydrofolate reductase (DHFR) of Escherichia coli (DDd). The development of DDds comprising various mutations relative to wild-type E. coli DHFR is well known in the art (see, e.g., Iwamoto et al. (2010). “A general chemical method to regulate protein stability in the mammalian central nervous system” Chem Biol, 17(9), 981-988, which is incorporated by reference herein). Such mutations are well known to, for instance, modify the folding of DHFR and therefore its susceptibility to protein degradation. The small-molecule ligand trimethoprim (TMP) and derivatives thereof stabilize DDd in a rapid, reversible, and dose-dependent manner. For reference, the amino acid sequence of E. coli strain K-12 DHFR comprising R12H and G67S mutations, upon which many DHFR variants are based, is provided in SEQ ID NO: 32.

In some embodiments, the destabilization domain is a destabilization domain derived from the ligand binding domain of human estrogen receptor (DDe). The development of DDes comprising various mutations relative to wild-type human estrogen receptor ligand binding domain is well known in the art (see, e.g., Miyazaki et al. (2012) “Destabilizing domains derived from the human estrogen receptor” J Am Chem Soc, 134(9):3942-5, which is incorporated by reference herein). Such mutations are well known to, for instance, modify the folding of the ligand binding domain and therefore its susceptibility to protein degradation. The small-molecule ligands CMP8 or 4 hydroxytamoxifen (4-OHT) and derivatives thereof stabilize DDe in a rapid, reversible, and dose-dependent manner. For reference, the amino acid sequence of Homo sapiens estrogen receptor ligand binding domain comprising T371A, L384M, M421G, N519S, G521R, and Y537S mutations, upon which many estrogen receptor ligand binding domain variants are based, is provided in SEQ ID NO: 33.

In some embodiments, the destabilization domain is a destabilization domain derived from the human FK506 binding protein (FKBP) (DDf). The development of DDfs comprising various mutations relative to wild-type human FKBP is well known in the art (see, e.g., Banaszynski et al. (2006) “A rapid, reversible, and tunable method to regulate protein function in living cells using synthetic small molecules” Cell, 126(5), 995-1004, which is incorporated by reference herein). Such mutations are well known to, for instance, modify the folding of FKBP and therefore its susceptibility to protein degradation. The small-molecule ligand Shield-1 and derivatives thereof stabilize DDf in a rapid, reversible, and dose-dependent manner. For reference, the amino acid sequence of Homo sapiens FKBP comprising a F36V mutation, upon which many FKBP variants are based, is provided in SEQ ID NO: 34.

In some embodiments, a destabilization domain of a phosphatase regulator is stabilized in the presence of one or more molecules, such that the susceptibility of the protein to components of protein degradation machinery is reduced. In some embodiments, the molecule is a small molecule, generally understood in the art to be any molecule with a molecular mass of less than 900 Daltons. In some embodiments, a small molecule is cell permeable. In some embodiments, addition of a small molecule to a cell comprising a feedback controller circuit encoding a phosphatase regulator comprising a destabilization domain causes the intracellular level of the phosphatase regulator to increase relative to the absence of the small molecule. In such embodiments, the intracellular level of the phosphatase regulator may be increased by 10% or more, increased by 20% or more, increased by 30% or more, increased by 40% or more, increased by 50% or more, increased by 60% or more, increased by 70% or more, increased by 80% or more, increased by 90% or more, increased by 100% or more, increased by 125% or more, increased by 150% or more, increased by 175% or more, increased by 200% or more, increased by 250% or more, increased by 300% or more, increased by 350% or more, increased by 400% or more, increased by 450% or more, increased by 500% or more, increased by 600% or more, increased by 700% or more, increased by 800% or more, increased by 900% or more, or increased by 1000% or more.

In some embodiments, the small molecule directly interacts (i.e., binds) with the destabilization domain. In some embodiments, interaction with the small molecule enhances folding of the destabilization domain. In some embodiments, interaction with the small molecule prevents recognition of the destabilization domain by components of protein degradation machinery. In some embodiments, the interaction between the destabilization domain and the small molecule may be characterized in terms of a dissociation constant (K_{D}). In such embodiments, the small molecule interacts with a destabilization domain with a K_{D} of at least 10 pM, at least 20 pM, at least 30 pM, at least 40 pM, at least 50 pM, at least 60 pM, at least 70 pM, at least 80 pM, at least 90 pM, at least 100 pM, at least 125 pM, at least 150 pM, at least 175 pM, at least 200 pM, at least 250 pM, at least 300 pM, at least 350 pM, at least 400 pM, at least 450 pM, at least 500 pM, at least 600 pM, at least 700 pM, at least 800 pM, at least 900 pM, at least 1 nM, at least 10 nM, at least 25 nM, at least 50 nM, at least 75 nM, at least 100 nM, at least 125 nM, at least 150 nM, at least 175 nM, at least 200 nM, at least 250 nM, at least 300 nM, at least 350 nM, at least 400 nM, at least 450 nM, at least 500 nM, at least 600 nM, at least 700 nM, at least 800 nM, at least 900 nM, or at least 1 μM.

In some embodiments where at least one destabilization domain comprised by a phosphatase regulator is an E. coli dihydrofolate reductase (DHFR) destabilization domain (DDd), the small molecule is trimethoprim (TMP) or a derivative thereof. Derivatives of trimethoprim are compounds that would generally be understood by those well versed in the art to share structural features of trimethoprim and include, for instance, iodinated trimethoprim (TMP-I) and diaveridine (see, e.g., Nilchan et al. (2018) “Halogenated trimethoprim derivatives as multidrug-resistant Staphylococcus aureus therapeutics” Bioorg Med Chem, 26(19):5343-5348, which is incorporated by reference herein). The use of trimethoprim and derivatives thereof to reduce degradation of proteins containing a DDd is well known in the art, for example, in Iwamoto et al. (2010). “A general chemical method to regulate protein stability in the mammalian central nervous system” Chem Biol, 17(9), 981-988, which is incorporated by reference herein.

In some embodiments where at least one destabilization domain comprised by a phosphatase regulator is a human estrogen receptor ligand binding domain destabilization domain (DDe), the small molecule is 4-hydroxytamoxifen (4-OHT) or a derivative thereof. Derivatives of 4-hydroxytamoxifen are compounds that would generally be understood by those well versed in the art to share structural features of 4-hydroxytamoxifen and include, for example, endoxifen (see, e.g., Maximov et al. (2018) “Endoxifen, 4-Hydroxytamoxifen and an Estrogenic Derivative Modulate Estrogen Receptor Complex Mediated Apoptosis in Breast Cancer” Mol Pharmacol, 94(2), 812-822, which is incorporated by reference herein). The use of 4-hydroxytamoxifen and derivatives thereof to reduce degradation of proteins containing a DDe is well known in the art, for example, in Miyazaki et al. (2012) “Destabilizing domains derived from the human estrogen receptor” J Am Chem Soc, 134(9):3942-5, which is incorporated by reference herein.

In some embodiments where at least one destabilization domain comprised by a phosphatase regulator is a human FK506 binding protein (FKBP) destabilization domain (DDf), the small molecule is a Shield ligand or a derivative thereof. A Shield ligand may be, for example, Shield-1 or Shield-2 (see, e.g., Grimley et al. (2008) “Synthesis and analysis of stabilizing ligands for FKBP-derived destabilizing domains”. Bioorg Med Chem Lett, 18(2), 759-761, which is incorporated by reference herein), or a derivative compound that would generally be understood by those well versed in the art to share structural features of Shield-1 or Shield-2. The use of Shield ligands to reduce degradation of proteins containing a DDf is well known in the art, for example, in Banaszynski et al. (2006) “A rapid, reversible, and tunable method to regulate protein function in living cells using synthetic small molecules”. Cell, 126(5), 995-1004, which is incorporated by reference herein.

In some embodiments, the destabilization domain is selected from PEST, a destabilization domain from E. coli dihydrofolate reductase, or a destabilization domain derived from human FK506-binding protein (FKBP) or FKBP-rapamycin-binding (FRB) protein. A PEST domain comprises an amino acid sequence that is rich in proline (P), glutamic acid (E), serine (S), and threonine (T). PEST sequences trigger degradation of proteins containing them, and thus proteins containing PEST sequences are less stable in cells that do not have PEST sequences. In the absence of a stabilizing molecule, the protein containing the destabilization domain is readily degraded in cells, preventing it from contributing to replication. Thus, expression of the phosphatase regulator, and thus the extent of tuning of output molecule expression, can be selectively controlled by adding the stabilizing molecule to a system, or prevented by withholding the stabilizing molecule from the system. In some embodiments, the stabilizing molecule is TMP, OHT-1, rapamycin, or a rapalog.

In some embodiments, the degradation domain is selected from the group consisting of PEST, DDd, DDe, and DDf. In some embodiments, the degradation domain is PEST. In some embodiments, the degradation domain is DDd. In some embodiments, the degradation domain is DDe. In some embodiments, the degradation domain is DDf. In some embodiments, the phosphatase regulator comprises one or more degradation domains at the N-terminus. In some embodiments, the phosphatase regulator comprises one or more degradation domains at the N-terminus, and does not comprise a degradation domain at the C-terminus. Quite unexpectedly, it was determined that fusing a degradation domain to the N-terminus was more effective, compared to fusion at the C-terminus, at destabilizing the phosphatase regulator in a manner that could be prevented by the cognate small molecule. These results were surprising, as a person of ordinary skill in the art would not reasonably have expected the efficiency of destabilization by the same degradation domain to vary depending on where it was attached to the base protein. In embodiments of phosphatase regulators comprising multiple degradation domains, the multiple degradation domains may be the same degradation domain, or different degradation domains. In some embodiments, a phosphatase regulator comprises identical degradation domains at the N-terminus. Identical degradation domains are degradation domains that are capable of being stabilized by the same molecule, while two different degradation domains are not capable of being stabilized by the same molecule. Thus, one molecule is sufficient to stabilize a protein comprising two or more identical degradation domains, while a protein comprising different degradation domains must be stabilized by exposure to a cognate stabilizing molecule corresponding to each different degradation domain. In some embodiments, a phosphatase regulator comprises a first degradation domain at the N-terminus and a different degradation domain at the N-terminus of the first degradation domain, or between the first degradation domain and another domain of the fusion protein.

In some embodiments, the phosphatase regulator of the feedback controller circuits provided herein comprises: (i) a truncated EnvZ domain comprising a DHp and CA domain of the EnvZ, a substitution in the CA domain corresponding to an N343K substitution of the amino acid sequence of SEQ ID NO: 1, (ii) a leucine zipper domain linked to the N-terminus of the truncated EnvZ domain; and (iii) a degradation domain linked to the N-terminus of the leucine zipper domain. In some embodiments, the phosphatase regulator comprises: (i) a truncated EnvZ domain comprising a DHp and CA domain of the EnvZ, a substitution in the CA domain corresponding to an N343K substitution of the amino acid sequence of SEQ ID NO: 1, (ii) a GCN4 leucine zipper domain linked to the N-terminus of the truncated EnvZ domain; and (iii) a DDd degradation domain linked to the N-terminus of the leucine zipper domain. In some embodiments, the phosphatase regulator comprises an amino acid sequence with at least 80%, at least 85%, at least 90%, at least 95%, at least 97%, at least 98%, or at least 99% sequence identity to the amino acid sequence of SEQ ID NO: 30. In some embodiments, the phosphatase regulator comprises the amino acid sequence of SEQ ID NO: 30.

In some embodiments, the phosphatase regulator of the feedback controller circuits provided herein comprises a plurality of monomers, with each monomer comprising a fusion protein that comprises a phosphatase domain, a leucine zipper domain linked to the phosphatase domain, and one or more degradation domains linked to the phosphatase domain and/or leucine zipper domain. In some embodiments, each monomer comprises: (i) a truncated EnvZ domain comprising a DHp and CA domain of the EnvZ, a substitution in the CA domain corresponding to an N343K substitution of the amino acid sequence of SEQ ID NO: 1, (ii) a leucine zipper domain linked to the N-terminus of the truncated EnvZ domain; and (iii) a degradation domain linked to the N-terminus of the leucine zipper domain. In some embodiments, each monomer comprises: (i) a truncated EnvZ domain comprising a DHp and CA domain of the EnvZ, a substitution in the CA domain corresponding to an N343K substitution of the amino acid sequence of SEQ ID NO: 1, (ii) a GCN4 leucine zipper domain linked to the N-terminus of the truncated EnvZ domain; and (iii) a DDd degradation domain linked to the N-terminus of the leucine zipper domain. In some embodiments, each monomer comprises an amino acid sequence with at least 80%, at least 85%, at least 90%, at least 95%, at least 97%, at least 98%, or at least 99% sequence identity to the amino acid sequence of SEQ ID NO: 30. In some embodiments, each monomer comprises the amino acid sequence of SEQ ID NO: 30.

An “amino acid substitution” without the reference to a specific amino acid, may include any amino acid other than the wild-type residue normally found at that position. Such substitutions may be replacement with non-polar (hydrophobic) amino acids, such as glycine, alanine, valine, leucine, isoleucine, methionine, phenylalanine, tryptophan, and proline. Substitutions may be replacement with polar (hydrophilic) amino acids such as serine, threonine, cysteine, tyrosine, asparagine, and glutamine. Substitutions may be replacement with electrically charged amino acids, e.g., negatively electrically charged amino acids such as aspartic acid and glutamic acid and positively electrically charged amino acids such as lysine, arginine, and histidine.

The substitution mutations described herein will typically be replacement with a different naturally occurring amino acid residue, but in some cases non-naturally occurring amino acid residues may also be used for the substitution. Non-natural amino acids, as the term is used herein, are non-proteinogenic (i.e., non-protein coding) amino acids that either occur naturally or are chemically synthesized. Examples include but are not limited to β-amino acids (β3 and (β2), homo-amino acids, proline and pyruvic acid derivatives, 3-substituted alanine derivatives, glycine derivatives, ring-substituted phenylalanine and tyrosine derivatives, linear core amino acids, di-amino acids, D-amino acids, and N-methyl amino acids.

The term “identity” refers to the overall relatedness between biological molecule, for example, polypeptide molecules. Calculation of the percent identity of two polypeptide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. Exemplary computer software to determine homology between two sequences include, but are not limited to BLASTP, CLUSTAL, and MAFFT.

In some embodiments, the activator encoded by the signal circuit comprises a response regulator (RR) of the bacterial two-component system. In some embodiments, the activator comprises the RR fused to and activation domain. An “activation domain,” as used herein, refers to a protein or protein domain that in conjunction with a DNA binding domain (e.g., the RR of the present disclosure), can activate transcription from a promoter. Any activation domains known in the art may be used in accordance with the present disclosure. Non-limiting examples of activation domains include: VP16, VP64, p65, and VPR and exemplary sequences are provided in Table 2. “Fuse” means to connect two different protein partners, e.g., via an amide bond, thus to form a fusion protein. “Linking” two proteins to form a fusion protein may be achieved by acid sequences via an amide bond with no intervening amino acids, or by connecting two different protein domains or subunits (e.g., two activation domains, an activation domain and a response regulator, a phosphatase domain and a dimerization domain, a dimerization domain and a degradation domain) by an intervening amino acid sequence, referred to herein as a “peptide linker.” In some embodiments, a linker has a length of (or has a length of at least) 3, 4, 5, 6, 7, 8, 9, 10, 11, 12, 13, 14, 15, 16, 17, 18, 19, or 20 amino acids. In some embodiments, a linker is (or is at least) amino acids long (e.g., GGSGGSGGSGGSGGG (SEQ ID NO: 35)). In some embodiments, a linker is (or is at least) 8 amino acids long (e.g., GGGSGGGS (SEQ ID NO: 36)). In some embodiments, a linker is (or is at least) 7 amino acids long (e.g., GGGSGGG (SEQ ID NO: 37)). In some embodiments, a linker is (or is at least) 4 amino acid long (e.g., GGGS (SEQ ID NO: 38)). In some embodiments, the linker comprises (GGGS). (SEQ ID NO: 39), where n is any integer from 1-5. In some embodiments, a linker is (or is at least) 4 amino acid long (e.g., GSGG (SEQ ID NO: 40)). In some embodiments, the linker comprises (GSGG). (SEQ ID NO: 41), where n is any integer from 1-5. In some embodiments, a linker is a glycine linker, for example having a length of (or a length of at least) 3 amino acids (e.g., GGG). In some embodiments, a fusion protein includes two or more linkers, which may be the same or different from each other. In some embodiments, two domains of a fusion protein are fused without a linker, while other domains are linked by a peptide linker. In some embodiments, the RR is fused at the N terminus of the activation domain. In some embodiments, the RR is fused at the C-terminus of the activation domain.

TABLE 2
Exemplary activation domains

| Activation | |
|---|---|
| Domain | Amino Acid Sequence |
| VP16 | APPTDVSLGDELHLDGEDVAMAHADALDDFDLDMLGDGDSPGPGFTPHDSAPYGALDMADFEFEQM |
| | FTDALGIDEYGG (SEQ ID NO: 42) |
| VP64 | EASGSGRADALDDFDLDMLGSDALDDFDLDMLGSDALDDFDLDMLGSDALDDFDLDMLINSR |
| | (SEQ ID NO: 43) |
| P65 | SQYLPDTDDRHRIEEKRKRTYETFKSIMKKSPFSGPTDPRPPPRRIAVPSRSSASVPKPAPQPYPF |
| | TSSLSTINYDEFPTMVFPSGQISQASALAPAPPQVLPQAPAPAPAPAMVSALAQAPAPVPVLAPGP |
| | PQAVAPPAPKPTQAGEGTLSEALLQLQFDDEDLGALLGNSTDPAVFTDLASVDNSEFQQLLNQGIP |
| | VAPHTTEPMLMEYPEAITRLVTGAQRPPDPAPAPLGAPGLPNGLLSGDEDFSSIADMDFSALL |
| | (SEQ ID NO: 44) |
| VPR | RADALDDFDLDMLGSDALDDFDLDMLGSDALDDFDLDMLGSDALDDEDLDMLINSRSSGSPKKKRK |
| | VGSQYLPDTDDRHRIEEKRKRTYETFKSIMKKSPFSGPTDPRPPPRRIAVPSRSSASVPKPAPQPY |
| | PFTSSLSTINYDEFPTMVFPSGQISQASALAPAPPQVLPQAPAPAPAPAMVSALAQAPAPVPVLAP |
| | GPPQAVAPPAPKPTQAGEGTLSEALLQLQFDDEDLGALLGNSTDPAVFTDLASVDNSEFQQLLNQG |
| | IPVAPHTTEPMLMEYPEAITRLVTGAQRPPDPAPAPLGAPGLPNGLLSGDEDFSSIADMDFSALLG |
| | SGSGSRDSREGMFLPKPEAGSAISDVFEGREVCQPKRIRPFHPPGSPWANRPLPASLAPTPTGPVH |
| | EPVGSLTPAPVPQPLDPAPAVTPEASHLLEDPDEETSQAVKALREMADTVIPQKEEAAICGQMDLS |
| | HPPPRGHLDELITTLESMTEDLNLDSPLTPELNEILDTFLNDECLLHAMHISTGLSIFDTSLF |
| | (SEQ ID NO: 45) |

Any RRs that are known in the art may be used in the activator of the present disclosure, including, without limitation: BaeR, BasR, CpxR, CusR, KdpE, NarL, OmpR, PhoP, PhoB, QseB, RstA, and YedW. In some embodiments, the response regulator is selected from the group consisting of BasR, CpxR, CusR, NarL, PhoB, RstA, and YedW. Exemplary amino acid sequences are provided in Table 3. It should be understood that the RRs are typically be used in conjunction with its cognate histidine kinase and phosphatase, since most bacterial two-component systems are orthogonal. For example, when the kinase and the phosphatase in the feedback controller circuit are derived from EnvZ, OmpR is used in the activator, e.g., to be used with an activation domain such as VP16, VP64, p65, or VPR, to form the activator encoded by the signal circuit. Similarly, when the kinase and the phosphatase in the feedback controller circuit are derived from NarX, NtrB, or PhoR, the activator comprises NarL, NtrC, or PhoB, respectively. In some embodiments, when the kinase and phosphatase are derived from EnvZ, the response regulator comprises OmpR. In some embodiments, when the kinase and phosphatase are derived from BasS, the response regulator comprises BasR. In some embodiments, when the kinase and phosphatase are derived from CpxA, the response regulator comprises CpxR. In some embodiments, when the kinase and phosphatase are derived from YedV, the response regulator comprises YedW. In some embodiments, when the kinase and phosphatase are derived from NarX, the response regulator comprises NarL. In some embodiments, when the kinase and phosphatase are derived from QseC, the response regulator comprises PhoB. In some embodiments, when the kinase and phosphatase are derived from PhoQ, the response regulator comprises RstA. In some embodiments, when the kinase and phosphatase are derived from CusS, the response regulator comprises YedW.

One skilled in the art is familiar with activation domains and methods of fusing the RR to the activation domain.

TABLE 3
Exemplary response regulators

| Response | |
|---|---|
| Regulator | Amino Acid Sequence |
| OmpR | MQENYKILVVDDDMRLRALLERYLTEQGFQVRSVANAEQMDRLLTRESFHLMVLDLMLPGEDGLS |
| | ICRRLRSQSNPMPIIMVTAKGEEVDRIVGLEIGADDYIPKPFNPRELLARIRAVLRRQANELPGA |
| | PSQEEAVIAFGKFKLNLGTREMFREDEPMPLTSGEFAVLKALVSHPREPLSRDKLMNLARGREYS |
| | AMERSIDVQISRLRRMVEEDPAHPRYIQTVWGLGYVFVPDGSKA (SEQ ID NO: 46) |
| NarL | MSNQEPATILLIDDHPMLRTGVKQLISMAPDITVVGEASNGEQGIELAESLDPDLILLDLNMPGM |
| | NGLETLDKLREKSLSGRIVVFSVSNHEEDVVTALKRGADGYLLKDMEPEDLLKALHQAAAGEMVL |
| | SEALTPVLAASLRANRATTERDVNQLTPRERDILKLIAQGLPNKMIARRLDITESTVKVHVKHML |
| | KKMKLKSRVEAAVWVHQERIF (SEQ ID NO: 47) |
| NtrC | MQRGIVWVVDDDSSIRWVLERALAGAGLTCTTFENGAEVLEALASKTPDVLLSDIRMPGMDGLAL |
| | LKQIKQRHPMLPVIIMTAHSDLDAAVSAYQQGAFDYLPKPFDIDEAVALVERAISHYQEQQQPRN |
| | VQLNGPTTDIIGEAPAMQDVFRIIGRLSRSSISVLINGESGTGKELVAHALHRHSPRAKAPFIAL |
| | NMAAIPKDLIESELFGHEKGAFTGANTIRQGRFEQADGGTLFLDEIGDMPLDVQTRLLRVLADGQ |
| | FYRVGGYAPVKVDVRIIAATHQNLEQRVQEGKFREDLFHRLNVIRVHLPPLRERREDIPRLARHF |
| | LQVAARELGVEAKLLHPETEAALTRLAWPGNVRQLENTCRWLTVMAAGQEVLIQDLPGELFESTV |
| | AESTSQMQPDSWATLLAQWADRALRSGHQNLLSEAQPELERTLLTTALRHTQGHKQEAARLLGWG |
| | RNTLTRKLKELGME (SEQ ID NO: 48) |
| PhoB | MARRILVVEDEAPIREMVCFVLEQNGFQPVEAEDYDSAVNQLNEPWPDLILLDWMLPGGSGIQFI |
| | KHLKRESMTRDIPVVMLTARGEEEDRVRGLETGADDYITKPFSPKELVARIKAVMRRISPMAVEE |
| | VIEMQGLSLDPTSHRVMAGEEPLEMGPTEFKLLHFFMTHPERVYSREQLLNHVWGINVYVEDRTV |
| | DVHIRRLRKALEPGGHDRMVQTVRGTGYRFSTRF (SEQ ID NO: 49) |
| BasR | MKILIVEDDTLLLQGLILAAQTEGYACDSVTTARMAEQSLEAGHYSLVVLDLGLPDEDGLHFLAR |
| | IRQKKYTLPVLILTARDTLTDKIAGLDVGADDYLVKPFALEELHARIRALLRRHNNQGESELIVG |
| | NLTLNMGRRQVWMGGEELILTPKEYALLSRLMLKAGSPVHREILYNDIYNWDNEPSTNTLEVHIH |
| | NLRDKVGKARIRTVRGFGYMLVANEEN (SEQ ID NO: 50) |
| CpxR | MNKILLVDDDRELTSLLKELLEMEGFNVIVAHDGEQALDLLDDSIDLLLLDVMMPKKNGIDTLKA |
| | LRQTHQTPVIMLTARGSELDRVLGLELGADDYLPKPENDRELVARIRAILRRSHWSEQQQNNDNG |
| | SPTLEVDALVLNPGRQEASFDGQTLELTGTEFTLLYLLAQHLGQVVSREHLSQEVLGKRLTPFDR |
| | AIDMHISNLRRKLPDRKDGHPWFKTLRGRGYLMVSAS (SEQ ID NO: 51) |
| CusR | MKLLIVEDEKKTGEYLTKGLTEAGFVVDLADNGLNGYHLAMTGDYDLIILDIMLPDVNGWDIVRM |
| | LRSANKGMPILLLTALGTIEHRVKGLELGADDYLVKPFAFAELLARVRILLRRGAAVIIESQFQV |
| | ADLMVDLVSRKVTRSGTRITLTSKEFTLLEFFLRHQGEVLPRSLIASQVWDMNFDSDTNAIDVAV |
| | KRLRGKIDNDFEPKLIQTVRGVGYMLEVPDGQ (SEQ ID NO: 52) |
| RstA | MNTIVFVEDDAEVGSLIAAYLAKHDMQVTVEPRGDQAEETILRENPDLVLLDIMLPGKDGMTICR |
| | DLRAKWSGPIVLLTSLDSDMNHILALEMGACDYILKTTPPAVLLARLRLHLRQNEQATLTKGLQE |
| | TSLTPYKALHFGTLTIDPINRVVTLANTEISLSTADFELLWELATHAGQIMDRDALLKNLRGVSY |
| | DGLDRSVDVAISRLRKKLLDNAAEPYRIKTVRNKGYLFAPHAWE (SEQ ID NO: 53) |
| YedW | MKILLIEDNQRTQEWVTQGLSEAGYVIDAVSDGRDGLYLALKDDYALIILDIMLPGMDGWQILQT |
| | LRTAKQTPVICLTARDSVDDRVRGLDSGANDYLVKPFSFSELLARVRAQLRQHHTLNSTLEISGL |
| | RMDSVSQSVSRDNISITLTRKEFQLLWLLASRAGEIIPRIVIASEIWGINFDSDTNTVDVAIRRL |
| | RAKVDDPFPEKLIATIRGMGYSFVAVKK (SEQ ID NO: 54) |

In some embodiments, the activatable promoter of the signal circuit comprises one or more (e.g., 1, 2, 3, 4, 5, 6, 7, 8, 9, 10, or more) response elements that bind to the activator, fused to a minimal promoter. A “minimal promoter” refers to the minimal elements of a promoter that has the promoter function. A minimal promoter typically contains the TATA box and transcription initiation site. Minimal promoters are typically in active unless regulatory elements that enhance promoter activity are placed upstream (e.g., such as the response elements to the RRs, as described herein). In some embodiments, the activatable promoter of the signal circuit comprises one or more (e.g., 1, 2, 3, 4, 5, or more) response elements that bind to the activator. In some embodiments, the activatable promoter of the signal circuit comprises three response elements that bind to the activator. A “response element” is a short sequences of DNA within a gene promoter region that are able to bind specific transcription factors and regulate transcription of genes. Under certain conditions, a transcription activator protein binds to the response element and stimulates transcription. Herein, the activator binds to the response elements in the activatable promoter of the signal circuit, activating the activatable promoter and producing the output molecule. In some embodiments, the activatable promoter comprises one or more (e.g., 1, 2, 3, 4, 5, or more) response elements fused to a minimal protein at the 5′ end. In some embodiments, the one or more (e.g., 1, 2, 3, 4, 5, or more) response elements are connected without a nucleotide linker between each response element. In some embodiments, the one or more (e.g., 1, 2, 3, 4, 5, or more) response elements are connected with a nucleotide linker between each response element. In some embodiments, the linker is 2-20 nucleotides long. For example, the linker may be 2, 3, 4, 5, 6, 7, 8, 9, 10, 11, 12, 13, 14, 15, 16, 17, 18, 19, or 20 nucleotides long. Longer or shorter linkers may also be used.

In some embodiments, each of the response element comprises one or more (e.g., 1, 2, 3, 4, 5, 6, 7, 8, 9, 10, or more) operators. In some embodiments, each of the response element comprises 3 operators. An “operator,” as used herein, refers to a segment of DNA to which a repressor binds to regulate gene expression by repressing it. In the lac operon, an operator is defined as a segment between the promoter and the genes of the operon. When bound by a repressor, the repressor protein physically obstructs the RNA polymerase from transcribing the genes, thus repressing transcription of the gene.

In some embodiments, the one or more (e.g., 1, 2, 3, 4, 5, 6, 7, 8, 9, 10, or more) operators are connected without a nucleotide spacer between each operator. In some embodiments, the one or more (e.g., 1, 2, 3, 4, 5, 6, 7, 8, 9, 10, or more) operators are connected with a nucleotide spacer between each operator. In some embodiments, the nucleotide spacer is 2-10 nucleotide long. For example, the nucleotide spacer may be 2, 3, 4, 6, 7, 8, 9, or 10 nucleotides long. Longer or shorter nucleotide spacers may also be used. Non-limiting, exemplary RRs and their respective operators are provided in Table 4.

TABLE 4
Exemplary response regulator operator sequences

| Name of | | SEQ |
|---|---|---|
| Response | | |
| Regulator | Operator Sequence | ID NO. |
| OmpR | ATTTACATTTTGAAACATCTA | 55 |
| NarL | TACCGCTATTGAGGTA | 56 |
| NtrC | TGCACTAAAATGGTGCA | 57 |
| PhoB | CTGTCATAWAWCTGTCAY | 58 |
| | (W is A or T, Y is C or T) | |

In some embodiments, the feedback controller circuit of the present disclosure further comprises a control circuit. A “control circuit” refers to a circuit that produces a constant signal independent of the input (e.g., the microRNA profile of a cell) and may be used to control for variations caused by other factors other than the microRNA profile, e.g., transfection, cellular health, etc. The control circuit comprises a constitutive promoter operably linked to a nucleotide sequence encoding a control signal that is different from the first output molecule or the second output molecule. The control signal is typically a detectable molecule such as a fluorescent molecule.

### Genetic Elements of Feedback Controller Circuits

Further provided herein are the various genetic elements used in the genetic circuits of the feedback controller circuits of the present disclosure. A “genetic element” refers to a particular nucleotide sequence that has a role in nucleic acid expression (e.g., promoter, enhancer, terminator) or encodes a discrete product of a genetic circuit (e.g., an activator, a microRNA, or an output molecule).

An “activator,” as used herein, refers to a transcriptional activator. The terms “activator” or “transcriptional activator” are used interchangeably herein. A transcriptional activator is a protein that increases gene transcription of a gene or set of genes. Most activators function by binding sequence-specifically to a DNA site located in or near a promoter and making protein—protein interactions with the general transcription machinery (RNA polymerase and general transcription factors), thereby facilitating the binding of the general transcription machinery to the promoter. In a feedback controller circuit comprising a first genetic circuit encoding an activator, and a second genetic circuit comprising an activatable promoter that is capable of being activated by the activator, transcription of mRNA encoded by the second genetic circuit is dependent on expression of the activator by the first genetic circuit. Thus, the first genetic circuit is said to transcriptionally regulate the second genetic circuit.

Herein, the expression of a gene is considered to be “activated” by an activator if the expression of the genes is at least 20% higher in the presence of the activator, compared to without the activator. For example, the expression of a gene is considered to be activated by an activator if the expression of the genes is at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold, or higher in the presence of the activator, compared to without the activator. In some embodiments, the expression of a gene is considered to be activated by an activator if the expression of the genes is at least 20%, 30%, 40%, 50%, 60%, 70%, 80%, 90%, 100%, 2-fold, 5-fold, 10-fold, 100-fold, 1000-fold, or higher in the presence of the activator, compared to without the activator.

One skilled in the art is able to choose the transcriptional activators or repressors for use in accordance with the present disclosure. Public databases are available for known or predicted transcriptional regulators, e.g., transcriptionfactor.org.

An “output molecule,” as used herein, refers to a signal produced by the feedback controller circuit after detecting the microRNA profile (e.g., a matching microRNA profile). The feedback controller circuit of the present disclosure is designed such that the output molecule is expressed when the kinase encoded by the input circuit phosphorylates the activator, with the phosphorylated activator promoting transcription of RNA encoding the output molecule. In some embodiments, the output molecule has a basal expression level and the expression level increases (e.g., by at least 20%) when the kinase is expressed, compared to when the kinase is not expressed. For example, the expression level of the output molecule may be at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold, or higher when a kinase is expressed, compared to when the kinase is not expressed. In some embodiments, the expression level of the output molecule is at least 20%, 30%, 40%, 50%, 60%, 70%, 80%, 90%, 100%, 2-fold, 5-fold, 10-fold, 100-fold, 1000-fold, or higher when a kinase is expressed, compared to when the kinase is not expressed.

The output molecule, in some embodiments, is a detectable protein. In some embodiments, a detectable protein is a fluorescent protein. A fluorescent protein is a protein that emits a fluorescent light when exposed to a light source at an appropriate wavelength (e.g., light in the blue or ultraviolet range). Suitable fluorescent proteins that may be used in accordance with the present disclosure include, without limitation, eGFP, eYFP, eCFP, mKate2, mCherry, mPlum, mGrape2, mRaspberry, mGrape 1, mStrawberry, mTangerine, mBanana, and mHoneydew. In some embodiments, a detectable protein is an enzyme that hydrolyzes an substrate to produce a detectable signal (e.g., a chemiluminescent signal). Such enzymes include, without limitation, beta-galactosidase (encoded by LacZ), horseradish peroxidase, or luciferase. In some embodiments, the output molecule is a fluorescent RNA. A fluorescent RNA is an RNA aptamer that emits a fluorescent light when bound to a fluorophore and exposed to a light source at an appropriate wavelength (e.g., light in the blue or ultraviolet range). Suitable fluorescent RNAs that may be used as an output molecule in the sensor circuit of the present disclosure include, without limitation, Spinach and Broccoli (e.g., as described in Paige et al., Science Vol. 333, Issue 6042, pp. 642-646, 2011, incorporated herein by reference).

In some embodiments, the output molecule is a therapeutic molecule. A “therapeutic molecule” is a molecule that has therapeutic effects on a disease or condition, and may be used to treat a diseases or condition. Therapeutic molecules of the present disclosure may be nucleic acid-based or protein or polypeptide-based.

In some embodiments, nucleic acid-based therapeutic molecule is an RNA interference (RNAi) molecule (e.g., a microRNA, siRNA, or shRNA) or an nucleic acid enzyme (e.g., a ribozyme). RNAi molecules and there use in silencing gene expression are familiar to those skilled in the art. In some embodiments, the RNAi molecule targets an oncogene. An oncogene is a gene that in certain circumstances can transform a cell into a tumor cell. An oncogene may be a gene encoding a growth factor or mitogen (e.g., c-Sis), a receptor tyrosine kinase (e.g., EGFR, PDGFR, VEGFR, or HER2/neu), a cytoplasmic tyrosine kinase (e.g., Src family kinases, Syk-ZAP-70 family kinases, or BTK family kinases), a cytoplasmic serine/threonine kinase or their regulatory subunits (e.g., Raf kinase or cyclin-dependent kinase), a regulatory GTPase (e.g., Ras), or a transcription factor (e.g., Myc). In some embodiments, the oligonucleotide targets Lipocalin (Lcn2) (e.g., a Lcn2 siRNA). One skilled in the art is familiar with genes that may be targeted for the treatment of cancer.

Non-limiting examples of protein or polypeptide-based therapeutic molecules include enzymes, regulatory proteins (e.g., immuno-regulatory proteins), antigens, antibodies or antibody fragments, and structural proteins. In some embodiments, the protein or polypeptide-based therapeutic molecules are for cancer therapy.

Suitable enzymes (for operably linking to a synthetic promoter) for some embodiments of this disclosure include, for example, oxidoreductases, transferases, polymerases, hydrolases, lyases, synthases, isomerases, and ligases, digestive enzymes (e.g., proteases, lipases, carbohydrases, and nucleases). In some embodiments, the enzyme is selected from the group consisting of lactase, beta-galactosidase, a pancreatic enzyme, an oil-degrading enzyme, mucinase, cellulase, isomaltase, alginase, digestive lipases (e.g., lingual lipase, pancreatic lipase, phospholipase), amylases, cellulases, lysozyme, proteases (e.g., pepsin, trypsin, chymotrypsin, carboxypeptidase, elastase), esterases (e.g. sterol esterase), disaccharidases (e.g., sucrase, lactase, beta-galactosidase, maltase, isomaltase), DNases, and RNases.

Non-limiting examples of antibodies and fragments thereof include: bevacizumab (AVASTIN®), trastuzumab (HERCEPTIN®), alemtuzumab (CAMPATH®, indicated for B cell chronic lymphocytic leukemia), gemtuzumab (MYLOTARG®, hP67.6, anti-CD33, indicated for leukemia such as acute myeloid leukemia), rituximab (RITUXAN®), tositumomab (BEXXAR®, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX®, indicated for ovarian cancer), edrecolomab (PANOREX®), daclizumab (ZENAPAX®), palivizumab (SYNAGIS®, indicated for respiratory conditions such as RSV infection), ibritumomab tiuxetan (ZEVALIN®, indicated for Non-Hodgkin's lymphoma), cetuximab (ERBITUX®), MDX-447, MDX-22, MDX-220 (anti-TAG-72), IOR-C5, IOR-T6 (anti-CD1), IOR EGF/R3, celogovab (ONCOSCINT® OV103), epratuzumab (LYMPHOCIDE®), pemtumomab (THERAGYN®), Gliomab-H (indicated for brain cancer, melanoma). In some embodiments, the antibody is an antibody that inhibits an immune check point protein, e.g., an anti-PD-1 antibody such as pembrolizumab (KEYTRUDA®) or nivolumab (OPDIVO®), or an anti-CTLA-4 antibody such as ipilimumab (YERVOY®). Other antibodies and antibody fragments may be operably linked to a synthetic promoter, as provided herein.

A regulatory protein may be, in some embodiments, a transcription factor or a immunoregulatory protein. Non-limiting, exemplary transcriptional factors include: those of the NFkB family, such as Rel-A, c-Rel, Rel-B, p50 and p52; those of the AP-1 family, such as Fos, FosB, Fra-1, Fra-2, Jun, JunB and JunD; ATF; CREB; STAT-1, -2, -3, -4, -5 and -6; NFAT-1, -2 and -4; MAF; Thyroid Factor; IRF; Oct-1 and -2; NF-Y; Egr-1; and USF-43, EGR1, Sp1, and E2F1. Other transcription factors may be operably linked to a synthetic promoter, as provided herein.

As used herein, an immunoregulatory protein is a protein that regulates an immune response. Non-limiting examples of immunoregulatory include: antigens, adjuvants (e.g., flagellin, muramyl dipeptide), cytokines including interleukins (e.g., IL-2, IL-7, IL-15 or superagonist/mutant forms of these cytokines), IL-12, IFN-gamma, IFN-alpha, GM-CSF, FLT3-ligand), and immunostimulatory antibodies (e.g., anti-CTLA-4, anti-CD28, anti-CD3, or single chain/antibody fragments of these molecules). Other immunoregulatory proteins may be operably linked to a synthetic promoter, as provided herein.

As used herein, an antigen is a molecule or part of a molecule that is bound by the antigen-binding site of an antibody. In some embodiments, an antigen is a molecule or moiety that, when administered to or expression in the cells of a subject, activates or increases the production of antibodies that specifically bind the antigen. Antigens of pathogens are well known to those of skill in the art and include, but are not limited to parts (coats, capsules, cell walls, flagella, fimbriae, and toxins) of bacteria, viruses, and other microorganisms. Examples of antigens that may be used in accordance with the disclosure include, without limitation, cancer antigens, self-antigens, microbial antigens, allergens and environmental antigens. Other antigens may be operably linked to a synthetic promoter, as provided herein.

In some embodiments, the antigen of the present disclosure is a cancer antigen. A cancer antigen is an antigen that is expressed preferentially by cancer cells (i.e., it is expressed at higher levels in cancer cells than on non-cancer cells) and, in some instances, it is expressed solely by cancer cells. Cancer antigens may be expressed within a cancer cell or on the surface of the cancer cell. Cancer antigens that may be used in accordance with the disclosure include, without limitation, MART-1/Melan-A, gp100, adenosine deaminase-binding protein (ADAbp), FAP, cyclophilin b, colorectal associated antigen (CRC)-C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A 11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4 and MAGE-C5. The cancer antigen may be selected from the group consisting of GAGE-1, GAGE-2, GAGE-3, GAGE-4, GAGE-5, GAGE-6, GAGE-7, GAGE-8, and GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pme1117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-3, SSX-4, SSX-5, SCP-1 and CT-7, CD20 and c-erbB-2. Other cancer antigens may be operably linked to a synthetic promoter, as provided herein.

In some embodiments, a protein or polypeptide-based therapeutic molecule is a fusion protein. A fusion protein is a protein comprising two heterologous proteins, protein domains, or protein fragments, that are covalently bound to each other, either directly or indirectly (e.g., via a linker), via a peptide bond. In some embodiments, a fusion protein is encoded by a nucleic acid comprising the coding region of a protein in frame with a coding region of an additional protein, without intervening stop codon, thus resulting in the translation of a single protein in which the proteins are fused together.

A “promoter” refers to a control region of a nucleic acid sequence at which initiation and rate of transcription of the remainder of a nucleic acid sequence are controlled. A promoter drives expression or drives transcription of the nucleic acid sequence that it regulates. A promoter may also contain sub-regions at which regulatory proteins and molecules may bind, such as RNA polymerase and other transcription factors. Promoters may be constitutive, inducible, activatable, repressible, tissue-specific or any combination thereof. A promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.”

In some embodiments, a coding nucleic acid sequence is positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see U.S. Pat. No. 4,683,202 and U.S. Pat. No.

In some embodiments, a promoter is an “inducible promoter,” which refer to a promoter that is characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

The administration or removal of an inducer signal results in a switch between activation and inactivation of the transcription of the operably linked nucleic acid sequence. Thus, the active state of a promoter operably linked to a nucleic acid sequence refers to the state when the promoter is actively regulating transcription of the nucleic acid sequence (i.e., the linked nucleic acid sequence is expressed). Conversely, the inactive state of a promoter operably linked to a nucleic acid sequence refers to the state when the promoter is not actively regulating transcription of the nucleic acid sequence (i.e., the linked nucleic acid sequence is not expressed).

An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Inducible promoters of the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some embodiments, an inducer signal of the present disclosure is an N-acyl homoserine lactone (AHL), which is a class of signaling molecules involved in bacterial quorum sensing. Quorum sensing is a method of communication between bacteria that enables the coordination of group based behavior based on population density. AHL can diffuse across cell membranes and is stable in growth media over a range of pH values. AHL can bind to transcriptional activators such as LuxR and stimulate transcription from cognate promoters.

In some embodiments, an inducer signal of the present disclosure is anhydrotetracycline (aTc), which is a derivative of tetracycline that exhibits no antibiotic activity and is designed for use with tetracycline-controlled gene expression systems, for example, in bacteria.

In some embodiments, an inducer signal of the present disclosure is isopropyl β-D-1-thiogalactopyranoside (IPTG), which is a molecular mimic of allolactose, a lactose metabolite that triggers transcription of the lac operon, and it is therefore used to induce protein expression where the gene is under the control of the lac operator. IPTG binds to the lac repressor and releases the tetrameric repressor from the lac operator in an allosteric manner, thereby allowing the transcription of genes in the lac operon, such as the gene coding for beta-galactosidase, a hydrolase enzyme that catalyzes the hydrolysis of β-galactosides into monosaccharides. The sulfur (S) atom creates a chemical bond which is non-hydrolyzable by the cell, preventing the cell from metabolizing or degrading the inducer. IPTG is an effective inducer of protein expression, for example, in the concentration range of 100 μM to 1.0 mM. Concentration used depends on the strength of induction required, as well as the genotype of cells or plasmid used. If lacIq, a mutant that over-produces the lac repressor, is present, then a higher concentration of IPTG may be necessary. In blue-white screen, IPTG is used together with X-gal. Blue-white screen allows colonies that have been transformed with the recombinant plasmid rather than a non-recombinant one to be identified in cloning experiments.

Other inducible promoter systems are known in the art and may be used in accordance with the present disclosure.

In some embodiments, inducible promoters of the present disclosure are from prokaryotic cells (e.g., bacterial cells). Examples of inducible promoters for use prokaryotic cells include, without limitation, bacteriophage promoters (e.g. Pls1con, T3, T7, SP6, PL) and bacterial promoters (e.g., Pbad, PmgrB, Ptrc2, Plac/ara, Ptac, Pm), or hybrids thereof (e.g. PLlacO, PLtetO). Examples of bacterial promoters for use in accordance with the present disclosure include, without limitation, positively regulated E. coli promoters such as positively regulated σ70 promoters (e.g., inducible pBad/araC promoter, Lux cassette right promoter, modified lamdba Prm promote, plac Or2-62 (positive), pBad/AraC with extra REN sites, pBad, P(Las) TetO, P(Las) CIO, P(Rhl), Pu, FecA, pRE, cadC, hns, pLas, pLux), GS promoters (e.g., Pdps), σ32 promoters (e.g., heat shock) and σ54 promoters (e.g., glnAp2); negatively regulated E. coli promoters such as negatively regulated σ70 promoters (e.g., Promoter (PRM+), modified lamdba Prm promoter, TetR-TetR-4C P(Las) TetO, P(Las) CIO, P(Lac) IQ, RecA_DlexO_DLacO1, dapAp, FecA, Pspac-hy, pcI, plux-cI, plux-lac, CinR, CinL, glucose controlled, modified Pr, modified Prm+, FecA, Pcya, rec A (SOS), Rec A (SOS), EmrR_regulated, BetI_regulated, pLac_lux, pTet_Lac, pLac/Mnt, pTet/Mnt, LsrA/cI, pLux/cI, LacI, LacIQ, pLacIQ1, pLas/cI, pLas/Lux, pLux/Las, pRecA with LexA binding site, reverse BBa_R0011, pLacI/ara-1, pLacIq, rrnB P1, cadC, hns, PfhuA, pBad/araC, nhaA, OmpF, RcnR), σS promoters (e.g., Lutz-Bujard LacO with alternative sigma factor σ38), G32 promoters (e.g., Lutz-Bujard LacO with alternative sigma factor σ32), and σ54 promoters (e.g., glnAp2); negatively regulated B. subtilis promoters such as repressible B. subtilis σA promoters (e.g., Gram-positive IPTG-inducible, Xyl, hyper-spank) and σB promoters. Other inducible microbial promoters may be used in accordance with the present disclosure.

The different genetic circuits of the feedback controller circuits may be included in one or more (e.g., 2, 3, or more) nucleic acid molecules (e.g., vectors) and introduced into a cell. A “nucleic acid” is at least two nucleotides covalently linked together, and in some instances, may contain phosphodiester bonds (e.g., a phosphodiester “backbone”). A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine. Nucleic acids of the present disclosure may be produced using standard molecular biology methods (see, e.g., Green and Sambrook, Molecular Cloning, A Laboratory Manual, 2012, Cold Spring Harbor Press).

In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

In some embodiments, different genetic circuits of the feedback controller circuit are delivered to a cell on one or more (e.g., 2, 3, 4, 5, 6, 7, 8, 9, 10, or more) vectors. A “vector” refers to a nucleic acid (e.g., DNA) used as a vehicle to artificially carry genetic material (e.g., an engineered nucleic acid) into a cell where, for example, it can be replicated and/or expressed. In some embodiments, a vector is an episomal vector (see, e.g., Van Craenenbroeck K. et al. Eur. J. Biochem. 267, 5665, 2000, incorporated by reference herein). A non-limiting example of a vector is a plasmid. Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector (e.g., retroviral, adenoviral, adeno-association, helper-dependent adenoviral systems, hybrid adenoviral systems, herpes simplex, pox virus, lentivirus, Epstein-Barr virus). In some embodiments, the viral vector is derived from an adeno-associated virus (AAV). In some embodiments, the viral vector is derived from a herpes simplex virus (HSV).

The nucleic acids or vectors containing the genetic circuits of the feedback controller circuit may be delivered to a cell by any methods known in the art for delivering nucleic acids. For example, for delivering nucleic acids to a prokaryotic cell, the methods include, without limitation, transformation, transduction, conjugation, and electroporation. For delivering nucleic acids to a eukaryotic cell, methods include, without limitation, transfection, electroporation, and using viral vectors.

Cells containing the feedback controller circuits are also provided herein. A “cell” is the basic structural and functional unit of all known independently living organisms. It is the smallest unit of life that is classified as a living thing. Some organisms, such as most bacteria, are unicellular (consist of a single cell). Other organisms, such as humans, are multicellular.

In some embodiments, a cell for use in accordance with the present disclosure is a prokaryotic cell, which may comprise a cell envelope and a cytoplasmic region that contains the cell genome (DNA) and ribosomes and various sorts of inclusions. In some embodiments, the cell is a bacterial cell. As used herein, the term “bacteria” encompasses all variants of bacteria, for example, prokaryotic organisms and cyanobacteria. Bacteria are small (typical linear dimensions of around 1 micron), non-compartmentalized, with circular DNA and ribosomes of 70S. The term bacteria also includes bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are gram-negative cells, and in some embodiments, the bacterial cells are gram-positive cells. Examples of bacterial cells that may be used in accordance with the invention include, without limitation, cells from Yersinia spp., Escherichia spp., Klebsiella spp., Bordetella spp., Neisseria spp., Aeromonas spp., Francisella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp. In some embodiments, the bacterial cells are from Staphylococcus aureus, Bacillus subtilis, Clostridium butyricum, Brevibacterium lactofermentum, Streptococcus agalactiae, Lactococcus lactis, Leuconostoc lactis, Streptomyces, Actinobacillus actinobycetemcomitans, Bacteroides, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus planta rum, Streptococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechrornogenes, Streptomyces ghanaenis, Halobacterium strain GRB, or Halobaferax sp. strain Aa2.2.

In some embodiments, a cell for use in accordance with the present disclosure is a eukaryotic cell, which comprises membrane-bound compartments in which specific metabolic activities take place, such as a nucleus. Examples of eukaryotic cells for use in accordance with the invention include, without limitation, mammalian cells, insect cells, yeast cells (e.g., Saccharomyces cerevisiae) and plant cells. In some embodiments, the eukaryotic cells are from a vertebrate animal. In some embodiments, the cell is a mammalian cell. In some embodiments, the cell is a human cell. In some embodiments, the cell is from a rodent, such as a mouse or a rat. Examples of vertebrate cells for use in accordance with the present disclosure include, without limitation, reproductive cells including sperm, ova and embryonic cells, and non-reproductive cells, including kidney, lung, spleen, lymphoid, cardiac, gastric, intestinal, pancreatic, muscle, bone, neural, brain and epithelial cells. Stem cells, including embryonic stem cells, can also be used.

In some embodiments, the cell is a diseased cell. A “diseased cell,” as used herein, refers to a cell whose biological functionality is abnormal, compared to a non-diseased (normal) cell. In some embodiments, the diseased cell is a cancer cell.

### Cell State Classifiers

Some aspects of the present disclosure relate to cell state classifiers comprising a feedback controller circuit comprising herein. A “cell state classifier,” as used herein, refers to a system with multiple genetic circuits integrated together by transcriptional or translational control, which is able to sense a microRNA profile (e.g., one or more microRNAs) in a cell and produce an output molecule (e.g., a detectable molecule or a therapeutic molecule) accordingly. A “microRNA profile,” as used herein, refers to the expression levels of one or more microRNAs in a cell or a cell type. The microRNA profile may contain expression levels of microRNAs that have no expression or lower expression (e.g., at least 30% lower), and/or expression levels of microRNAs that express or have higher expression (e.g., at least 30% higher) in a cell or a cell type, compared to another cell or a different cell type, respectively. MicroRNAs that have no expression or lower expression are referred to herein as “microRNA-low” or “miR-low,” while microRNAs that express or have high expression are referred to herein as “microRNA-high” or “miR-high.”

In part, the cell state classifiers of the present disclosure are designed to detect an miRNA by incorporating target sites of the miRNA to be detected into different genetic circuits (e.g., input, tuning, and/or signal circuits), such that the miRNA to be detected can regulate translation of circuits containing a corresponding miRNA target site. Binding of the miRNA to a corresponding target site on the mRNA encoded by an input, tuning, and/or signal circuit causes degradation of the mRNA, thereby preventing translation of molecules encoded by the circuit (e.g., kinases, phosphatases, activators, repressors, and/or output molecules). Thus, expression of one or more output products of the cell state classifier is sensitive to the presence and/or absence of certain miRNAs in a cell, such that the cell can be classified as containing a given miRNA profile based on expression of the output molecule. Furthermore, cell state classifiers can express a therapeutic molecule selectively in cells having a given miRNA profile, thereby reducing or eliminating deleterious effects of output molecule expression in undesired cells.

Sensing of multiple inputs (e.g., microRNAs) simultaneously is enabled by coupling their detection to different portions of the genetic circuit, such that the output molecule is produced only when the correct input profile of miRNAs is detected. The cell state classifier may be used in various applications. In some embodiments, the cell state classifier described herein is used for the detection of a diseased cell (e.g., a cancer cell). In some embodiments, detection of the diseased cell (e.g., the cancer cell) is achieved via the expression of a detectable output molecule (e.g., a fluorescent protein) upon detection of a matching microRNA profile. As such, cell state classifiers of the present disclosure may be used for diagnosing a disease (e.g., cancer). In some embodiments, detection of the diseased cell (e.g., a cancer cell) is coupled with the expression of a therapeutic molecule for treating a disease (e.g., cancer). Further, to evaluate the performance of the cell state classifiers described herein, a large combinatorial library of circuit variants are generated and the performance of each circuit variant may be evaluated in living cell assays.

In some embodiments, the cell state classifiers described herein comprise various genetic circuits (also termed “circuits”) that perform different functions. A “genetic circuit” is a functional unit of the cell state classifier. The genetic circuits of the present disclosure may function in sensing the microRNA profile, producing output molecules, producing control signal, or regulating the signals sensed or produced by the cell state classifier.

In some embodiments, the cell state classifier comprises one or more (e.g., 1, 2, 3, 4, 6, 7, 8, 9, 10, or more) sensor circuits. A “sensor circuit” is a genetic circuit that detects the microRNA profile of the cell. Different types of sensor circuits are used in the cell state classifier for detecting microRNA-high and microRNA-low. Sensor circuits comprise microRNA target sites for the microRNAs to be detected.

In some embodiments, the cell state classifiers described herein comprise a first sensor circuit that detects a first microRNAs that does not express or expresses at a low (e.g., undetectable) level in a cell. Such a first microRNA is referred to as “microRNA-low” or “miR-low” herein. The first sensor circuit is also referred to interchangeably herein as the “microRNA-low sensor” or “miR-low sensor.” In some embodiments, the first sensor circuit comprises the input circuit encoding the kinase and activator, such that expression of the kinase and activator are inhibited in the presence of any of the first miRNAs. As described herein, the first sensor circuit comprises one or more (e.g., 1, 2, 3, 4, 5, 6, 7, 8, 9, 10, or more) target sites of the first microRNA (microRNA-low) to be detected. In some embodiments, one first sensor circuit is used for the detection of one or multiple (e.g., 2, 3, 4, 5, 6, 7, 8, 9, or more) microRNA-low.

In some embodiments, the first sensor circuit further comprises a constitutive promoter operably linked to a nucleotide sequence encoding an activator and a constitutive promoter operably linked to a nucleotide sequence encoding a kinase that phosphorylates the activator.

In some embodiments, in the first sensor circuit, the one or more (e.g., 1, 2, 3, 4, 5, 6, 7, 8, 9, 10, or more) target sites for the first microRNA are inserted into non-coding regions of the circuit. For example, such non-coding region may be upstream and/or downstream of the nucleotide sequence encoding the activator and/or the kinase, or between the nucleotide sequence encoding the activator and the nucleotide sequence encoding the kinase. In some embodiments, four target sites for the first microRNA are used at each insertion site.

In some embodiments, the cell state classifier described herein comprises a second sensor circuit that detects a second microRNA that expresses (e.g., expression level is detectable or high) in a cell. Such second microRNA are referred to as “microRNA-high” or “miR-high” herein. The second sensor circuit is also referred to interchangeably herein as the “microRNA-high sensor” or “miR-high sensor.” In some embodiments, the second sensor circuit comprises the tuning circuit encoding the phosphatase regulator, such that expression of the phosphatase regulator is inhibited in the presence of any of the second miRNAs. The second sensor circuit comprises one or more (e.g., 1, 2, 3, 4, 5, 6, 7, 8, 9, 10, or more) target sites of the second microRNA (microRNA-high), and a constitutive promoter operably linked to a nucleotide sequence encoding a phosphatase that de-phosphorylates the phosphorylated activator. A “phosphatase” is an enzyme that uses water to cleave a phosphoric acid monoester into a phosphate ion and an alcohol, a process known as “dephosphorylation.” Phosphatase enzymes are essential to many biological functions, because phosphorylation (e.g. by protein kinases) and dephosphorylation (by phosphatases) serve diverse roles in cellular regulation and signaling. Phosphatases that catalyze the removal of a phosphate group from an amino acid residue in a protein is referred to as a “protein phosphatase.”

In some embodiments, the one or more (e.g., 1, 2, 3, 4, 5, 6, 7, 8, 9, 10, or more) target sites for the second microRNA are placed in a non-coding region of the second sensor circuit, e.g., upstream and/or downstream of the nucleotide sequence encoding the phosphatase. In some embodiments, the target sites for the second microRNA are upstream of the nucleotide sequence encoding the phosphatase. In some embodiments, the target sites for the second microRNA are downstream of the nucleotide sequence encoding the phosphatase. In some embodiments, the target sites for the second microRNA are downstream and upstream of the nucleotide sequence encoding the phosphatase. In some embodiments, four target sites for the second microRNA are used at each insertion site.

In some embodiments, the constitutive promoters in the first and second sensor circuits are the same. In some embodiments, the constitutive promoters in the first and second sensor circuits are different.

In some embodiments cell state classifier described herein further comprises a signal circuit. A “signal circuit,” as used herein, refers to a genetic circuit that responds to the sensor circuits and in turn produces an output molecule. The signal circuit of the present disclosure comprises one or more (e.g., 1, 2, 3, 4, 5, 6, 7, 8, 9, 10, or more) target sites for the first microRNA (microRNA-low), and an activatable promoter operably linked to a nucleotide sequence encoding an output molecule. In some embodiments, in the signal circuit, the one or more (e.g., 1, 2, 3, 4, 5, 6, 7, 8, 9, 10, or more) target sites for the first microRNA are placed in a non-coding region of the signal circuit, e.g., upstream and/or downstream of the nucleotide sequence encoding the output molecule. In some embodiments, the target sites for the first microRNA are downstream of the nucleotide sequence encoding the output molecule. In some embodiments, the target sites for the first microRNA are upstream of the nucleotide sequence encoding the output molecule. In some embodiments, the target sites for the first microRNA are downstream and upstream of the nucleotide sequence encoding the output molecule. In some embodiments, 4 target sites for the first microRNA are used at each insertion site.

The cell state classifier of the present disclosure is designed such that the output molecule is expressed when a matching microRNA profile is detected. In some embodiments, the output molecule has a basal expression level and the expression level increases (e.g., by at least 20%) when a matching microRNA profile is detected, compared to when a non-matching microRNA profile is detected. For example, the expression level of the output molecule may be at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold, or higher when a matching microRNA profile is detected, compared to when a non-matching microRNA profile is detected. In some embodiments, the expression level of the output molecule is 20%, 30%, 40%, 50%, 60%, 70%, 80%, 90%, 100%, 2-fold, 5-fold, 10-fold, 100-fold, 1000-fold, or higher when a matching microRNA profile is detected, compared to when a non-matching microRNA profile is detected.

An “activatable promoter” is a promoter that can be activated (e.g., by an activator) to drive the expression of the nucleotide sequence that it is operably linked to. In the signal circuit, the activatable promoter is activated by the phosphorylated activator produced by the first sensor circuit. When a matching microRNA profile is present, the activatable promoter is activated and the output molecule is produced. In some embodiments, the output molecule is a detectable molecule. As such, detection of the output molecule is an indication that a matching miRNA profiling is present in a cell.

### Genetic Elements of Cell State Classifiers

Further provided herein are genetic elements of the cell state classifiers of the present disclosure. The first and second sensor circuits of the cell state classifier “senses” microRNAs via microRNA target sites present in the sensor circuits. A “microRNA” or “miRNA” is a small non-coding RNA molecule that functions in RNA silencing and post-transcriptional regulation of gene expression (e.g., as described in Ambros et al., Nature 431 (7006): 350-5, 2004; and Bartel et al., Cell. 136 (2): 215-33, 2004). A microRNA may be 15-30 nucleotides in length. For example, a microRNA may be 15-30, 15-25, 15-20, 20-30, or 25-30 nucleotides in length. In some embodiments, a microRNA is 16-24 nucleotides in length. In some embodiments, a microRNA is 20-24 nucleotides in length. In some embodiments, a microRNA is 15, 16, 17, 18, 19, 20, 21, 22, 23, 24, 25, 26, 27, 28, 29, or 30 nucleotides in length.

A “microRNA target site” is a nucleotide sequence that is complementary to the nucleotide sequence of the microRNA. Naturally, microRNA targeting sites exist in messenger RNAs (mRNA), typically in the 3′ untranslated regions of mRNAs. Binding of the microRNA to its target site in via sequence complementarity leads to silencing of an output molecule either via degrading the mRNA or suppressing translation of the mRNA (e.g., as described in Bartel et al., Cell 136 (2): 215-33 (2009), incorporated herein by reference) containing the microRNA binding sites. Herein, when microRNA target sites are referred in the context of the genetic circuits (i.e., in the context of DNA), it intends to mean the nucleotide sequence that encodes the microRNA target sites in the mRNA that is produced from the genetic circuit. Thus, a genetic circuit that comprises a target site for an miRNA is translationally regulated by the miRNA, such that the presence of the miRNA in a cell prevents translation of the product encoded by the genetic circuit. As described herein, designated microRNA target sites are placed either upstream or downstream, or both, of a coding sequence in genetic circuits. As such, when a mRNA is produced from the genetic circuit, the microRNA target sites are present in the 5′ UTR or 3′ UTR, or both 5′ and 3′ UTRs in the mRNA.

One skilled in the art is familiar with the mechanism of gene silencing by microRNAs. For example, in the cell state classifier of the present disclosure, if a microRNA is expressed and a sensor circuit (e.g., the first or second sensor circuit) comprise one or more (e.g., 1, 2, 3, 4, 5, 6, 7, 8, 9, 10, or more) targets sites of the microRNA (either upstream or downstream of the coding sequence, or both), the microRNA can bind to the target sites in the mRNA produced by the sensor circuit and mediate the degradation of the mRNA, thus reducing the expression of the protein encoded by the mRNA (translational control). In some embodiments, expression of the protein encoded by the mRNA is reduced by at least 2-fold, at least 5-fold, at least 10-fold, at least 20-fold, at least 30-fold, at least 40-fold, at least 50-fold, at least 60-fold, at least 70-fold, at least 80-fold, at least 90-fold, at least 99-fold, or more compared to when the microRNA is not present. In some embodiments, expression of the protein encoded by the mRNA is no more than 1%, no more than 5%, no more than 10%, no more than 20%, no more than 30%, no more than 40%, no more than 50%, no more than 60%, no more than 70%, no more than 80% of the output molecule when the microRNA is not present. In some embodiments, a higher/lower level of the microRNA results in a higher/lower decrease in the protein encoded by the mRNA containing the microRNA target sites.

Information about the sequences, origins, and functions of known microRNAs may be found in publicly available databases (e.g., mirbase.org/, all versions, as described in Kozomara et al., Nucleic Acids Res 2014 42:D68-D73; Kozomara et al., Nucleic Acids Res 2011 39:D152-D157; Griffiths-Jones et al., Nucleic Acids Res 2008 36:D154-D158; Griffiths-Jones et al., Nucleic Acids Res 2006 34:D140-D144; and Griffiths-Jones et al., Nucleic Acids Res 2004 32:D109-D111, including the most recently released version miRBase 21, which contains “high confidence” microRNAs). Non-limiting examples of microRNAs that are expressed in cells and are able to be detected by the cell state classifier are: FF4, FF5, let-7b, let-7c, let-7d, let-7e, let-7f, let-7g, let-7i, miR-100, miR-103, miR-106a, miR-107, miR-10a, miR-10b, miR-122, miR-125a, miR-125b, miR-126, miR-126*, miR-127-3p, miR-128a, miR-129, miR-133b, miR-135b, miR-137, miR-141, miR-143, miR-145, miR-146a, miR-146b, miR-148a, miR-149, miR-150, miR-155, miR-15a, miR-17-3p, miR-17-5p, miR-181a, miR-181b, miR-181c, miR-183, miR-184, miR-186, miR-187, miR-189, miR-18a, miR-190, miR-191, miR-192, miR-195, miR-197, miR-199a, miR-199a*, miR-19a, miR-19b, miR-200a, miR-200a*, miR-200b, miR-200c, miR-202, miR-203, miR-205, miR-20a, miR-21, miR-210, miR-216, miR-218, miR-22, miR-221, miR-222, miR-223, miR-224, miR-23a, miR-23b, miR-24, miR-25, miR-26a, miR-26b, miR-27a, miR-27b, miR-29a, miR-29b, miR-296-5p, miR-301, miR-302a, miR-302a*, miR-30a, miR-30b, miR-30c, miR-30d, miR-30e-3p, miR-30e-5p, miR-31, miR-320, miR-323, miR-324-5p, miR-326, miR-330, miR-331, miR-335, miR-346, miR-34a, miR-370, miR-372, miR-373, miR-373*, miR-497, miR-498, miR-503, miR-92, miR-93, miR-96, and miR-99a.

In some embodiments, the microRNA detected using the cell state classifier of the present disclosure is selected from: hsa-let-7a-2-3p, hsa-let-7a-3p, hsa-let-7a-5p, hsa-let-7b-3p, hsa-let-7b-5p, hsa-let-7c-5p, hsa-let-7d-3p, hsa-let-7d-5p, hsa-let-7e-3p, hsa-let-7e-5p, hsa-let-7f-1-3p, hsa-let-7f-2-3p, hsa-let-7f-5p, hsa-let-7g-3p, hsa-let-7g-5p, hsa-let-7i-5p, hsa-miR-1, hsa-miR-1-3p, hsa-miR-1-5p, hsa-miR-100-3p, hsa-miR-100-5p, hsa-miR-101-3p, hsa-miR-101-5p, hsa-miR-103a-2-5p, hsa-miR-103a-3p, hsa-miR-105-3p, hsa-miR-105-5p, hsa-miR-106a-3p, hsa-miR-106a-5p, hsa-miR-106b-3p, hsa-miR-106b-5p, hsa-miR-107, hsa-miR-10a-3p, hsa-miR-10a-5p, hsa-miR-10b-3p, hsa-miR-10b-5p, hsa-miR-1185-1-3p, hsa-miR-1185-2-3p, hsa-miR-1185-5p, hsa-miR-122a-5p, hsa-miR-1249-3p, hsa-miR-1249-5p, hsa-miR-124a-3p, hsa-miR-125a-3p, hsa-miR-125a-5p, hsa-miR-125b-1-3p, hsa-miR-125b-2-3p, hsa-miR-125b-5p, hsa-miR-126-3p, hsa-miR-126-5p, hsa-miR-12′7-3p, hsa-miR-1271-3p, hsa-miR-1271-5p, hsa-miR-1278, hsa-miR-128-1-5p, hsa-miR-128-2-5p, hsa-miR-128-3p, hsa-miR-1285-3p, hsa-miR-1285-5p, hsa-miR-1287-3p, hsa-miR-1287-5p, hsa-miR-129-1-3p, hsa-miR-129-2-3p, hsa-miR-129-5p, hsa-miR-1296-3p, hsa-miR-1296-5p, hsa-miR-1304-3p, hsa-miR-1304-5p, hsa-miR-1306-3p, hsa-miR-1306-5p, hsa-miR-1307-3p, hsa-miR-1307-5p, hsa-miR-130a-3p, hsa-miR-130b-3p, hsa-miR-130b-5p, hsa-miR-132-3p, hsa-miR-132-5p, hsa-miR-133a-3p, hsa-miR-133a-5p, hsa-miR-133b, hsa-miR-134-3p, hsa-miR-134-5p, hsa-miR-135a-3p, hsa-miR-135a-5p, hsa-miR-135b-3p, hsa-miR-135b-5p, hsa-miR-136-3p, hsa-miR-136-5p, hsa-miR-138-1-3p, hsa-miR-138-5p, hsa-miR-139-3p, hsa-miR-139-5p, hsa-miR-140-3p, hsa-miR-140-5p, hsa-miR-141-3p, hsa-miR-141-5p, hsa-miR-142-3p, hsa-miR-142-5p, hsa-miR-143-3p, hsa-miR-143-5p, hsa-miR-144-3p, hsa-miR-144-5p, hsa-miR-145-5p, hsa-miR-146a-3p, hsa-miR-146a-5p, hsa-miR-147a, hsa-miR-148a-3p, hsa-miR-148a-5p, hsa-miR-148b-3p, hsa-miR-148b-5p, hsa-miR-149-3p, hsa-miR-144-3p, hsa-miR-150-3p, hsa-miR-150-5p, hsa-miR-151a-3p, hsa-miR-151a-5p, hsa-miR-152-3p, hsa-miR-152-5p, hsa-miR-154-3p, hsa-miR-154-5p, hsa-miR-155-3p, hsa-miR-155-5p, hsa-miR-hsa-miR-15a-5p, hsa-miR-15b-3p, hsa-miR-15b-5p, hsa-miR-16-1-3p, hsa-miR-16-2-3p, hsa-miR-16-5p, hsa-miR-17-3p, hsa-miR-17-5p, hsa-miR-181a-3p, hsa-miR-181a-5p, hsa-miR-181b-2-3p, hsa-miR-181b-5p, hsa-miR-181c-5p, hsa-miR-181d-3p, hsa-miR-181d-5p, hsa-miR-182-3p, hsa-miR-182-5p, hsa-miR-183-3p, hsa-miR-183-5p, hsa-miR-185-3p, hsa-miR-185-5p, hsa-miR-186-3p, hsa-miR-186-5p, hsa-miR-188-3p, hsa-miR-188-5p, hsa-miR-18a-3p, hsa-miR-18a-5p, hsa-miR-18b-5p, hsa-miR-1908-3p, hsa-miR-1908-5p, hsa-miR-190a-3p, hsa-miR-190a-5p, hsa-miR-191-3p, hsa-miR-191-5p, hsa-miR-1910-3p, hsa-miR-1910-5p, hsa-miR-192-3p, hsa-miR-192-5p, hsa-miR-193a-3p, hsa-miR-193a-5p, hsa-miR-193b-3p, hsa-miR-193b-5p, hsa-miR-194-3p, hsa-miR-194-5p, hsa-miR-195-3p, hsa-miR-195-5p, hsa-miR-196a-3p, hsa-miR-196a-5p, hsa-miR-196b-3p, hsa-miR-196b-5p, hsa-miR-19′7-3p, hsa-miR-19′7-5p, hsa-miR-199a-3p, hsa-miR-199a-5p, hsa-miR-199b-3p, hsa-miR-199b-5p, hsa-miR-19a-3p, hsa-miR-19a-5p, hsa-miR-19b-1-5p, hsa-miR-19b-2-5p, hsa-miR-19b-3p, hsa-miR-200a-3p, hsa-miR-200a-5p, hsa-miR-200b-3p, hsa-miR-200b-5p, hsa-miR-200c-3p, hsa-miR-200c-5p, hsa-miR-202-3p, hsa-miR-202-5p, hsa-miR-203a-3p, hsa-miR-203a-5p, hsa-miR-204-5p, hsa-miR-208b-3p, hsa-miR-208b-5p, hsa-miR-20a-3p, hsa-miR-20a-5p, hsa-miR-20b-3p, hsa-miR-20b-5p, hsa-miR-21-5p, hsa-miR-210-3p, hsa-miR-210-5p, hsa-miR-211-3p, hsa-miR-211-5p, hsa-miR-2116-3p, hsa-miR-2116-5p, hsa-miR-212-3p, hsa-miR-214-3p, hsa-miR-215-5p, hsa-miR-217, JG miR-218-1-3p, hsa-miR-218-5p, hsa-miR-219a-1-3p, hsa-miR-219a-2-3p, hsa-miR-219a-5p, hsa-miR-219b-3p, hsa-miR-219b-5p, hsa-miR-22-3p, hsa-miR-22-5p, hsa-miR-221-3p, hsa-miR-221-5p, hsa-miR-222-3p, hsa-miR-222-5p, hsa-miR-223-3p, hsa-miR-223-5p, hsa-miR-23a-3p, hsa-miR-23a-5p, hsa-miR-23b-3p, hsa-miR-24-1-5p, hsa-miR-25-3p, hsa-miR-25-5p, hsa-miR-26a-1-3p, hsa-miR-26a-2-3p, hsa-miR-26a-5p, hsa-miR-26b-5p, hsa-miR-27a-3p, hsa-miR-27a-5p, hsa-miR-27b-3p, hsa-miR-27b-5p, hsa-miR-28-3p, hsa-miR-28-5p, hsa-miR-296-3p, hsa-miR-296-5p, hsa-miR-299-3p, hsa-miR-299-5p, hsa-miR-29a-3p, hsa-miR-29a-5p, hsa-miR-29b-1-5p, hsa-miR-29b-3p, hsa-miR-29c-3p, hsa-miR-301a-3p, hsa-miR-301a-5p, hsa-miR-301b-3p, hsa-miR-301b-5p, hsa-miR-302a-3p, hsa-miR-302a-5p, hsa-miR-302b-5p, hsa-miR-302c-3p, hsa-miR-302c-5p, hsa-miR-3065-3p, hsa-miR-3065-5p, hsa-miR-3074-3p, hsa-miR-hsa-miR-30a-3p, hsa-miR-30a-5p, hsa-miR-30b-3p, hsa-miR-30b-5p, hsa-miR-30c-1-3p, hsa-miR-30c-2-3p, hsa-miR-30c-5p, hsa-miR-30d-3p, hsa-miR-30d-5p, hsa-miR-30e-3p, hsa-miR-30e-5p, hsa-miR-31-3p, hsa-miR-31-5p, hsa-miR-3130-3p, hsa-miR-3130-5p, hsa-miR-3140-3p, hsa-miR-3140-5p, hsa-miR-3144-3p, hsa-miR-3144-5p, hsa-miR-3158-3p, hsa-miR-3158-5p, hsa-miR-32-3p, hsa-miR-32-5p, hsa-miR-320a, hsa-miR-323a-3p, hsa-miR-323a-5p, hsa-miR-324-3p, hsa-miR-324-5p, hsa-miR-326, hsa-miR-328-3p, hsa-miR-328-5p, hsa-miR-329-3p, hsa-miR-329-5p, hsa-miR-330-3p, hsa-miR-330-5p, hsa-miR-331-3p, hsa-miR-331-5p, hsa-miR-335-3p, hsa-miR-335-5p, hsa-miR-337-3p, hsa-miR-337-5p, hsa-miR-338-3p, hsa-miR-338-5p, hsa-miR-339-3p, hsa-miR-339-5p, hsa-miR-33a-3p, hsa-miR-33a-5p, hsa-miR-33b-3p, hsa-miR-33b-5p, hsa-miR-340-3p, hsa-miR-340-5p, hsa-miR-342-3p, hsa-miR-342-5p, hsa-miR-345-3p, hsa-miR-345-5p, hsa-miR-34a-3p, hsa-miR-34a-hsa-miR-34b-3p, hsa-miR-34b-5p, hsa-miR-34c-3p, hsa-miR-34c-5p, hsa-miR-3605-3p, hsa-miR-3605-5p, hsa-miR-361-3p, hsa-miR-361-5p, hsa-miR-3613-3p, hsa-miR-3613-5p, hsa-miR-3614-3p, hsa-miR-3614-5p, hsa-miR-362-3p, hsa-miR-362-5p, hsa-miR-363-3p, hsa-miR-363-5p, hsa-miR-365a-3p, hsa-miR-365a-5p, hsa-miR-365b-3p, hsa-miR-365b-5p, hsa-miR-369-3p, hsa-miR-369-5p, hsa-miR-370-3p, hsa-miR-370-5p, hsa-miR-374a-3p, hsa-miR-374a-5p, hsa-miR-374b-3p, hsa-miR-374b-5p, hsa-miR-375, hsa-miR-376a-2-5p, hsa-miR-376a-3p, hsa-miR-376a-5p, hsa-miR-376c-3p, hsa-miR-376c-5p, hsa-miR-377-3p, hsa-miR-377-5p, hsa-miR-378a-3p, hsa-miR-378a-5p, hsa-miR-379-3p, hsa-miR-379-5p, hsa-miR-381-3p, hsa-miR-381-5p, hsa-miR-382-3p, hsa-miR-382-5p, hsa-miR-409-3p, hsa-miR-409-5p, hsa-miR-411-3p, hsa-miR-411-5p, hsa-miR-412-3p, hsa-miR-421, hsa-miR-423-3p, hsa-miR-423-5p, hsa-miR-424-3p, hsa-miR-424-5p, hsa-miR-425-3p, hsa-miR-425-5p, hsa-miR-431-3p, hsa-miR-431-5p, hsa-miR-432-5p, hsa-miR-433-3p, hsa-miR-433-5p, hsa-miR-449a, hsa-miR-449b-5p, hsa-miR-450a-1-3p, hsa-miR-450a-2-3p, hsa-miR-450a-5p, hsa-miR-450b-3p, hsa-miR-450b-5p, hsa-miR-451a, hsa-miR-452-3p, hsa-miR-4524a-3p, hsa-miR-4524a-5p, hsa-miR-4536-3p, hsa-miR-4536-5p, hsa-miR-454-3p, hsa-miR-454-5p, hsa-miR-4707-3p, hsa-miR-4707-5p, hsa-miR-4755-3p, hsa-miR-4755-5p, hsa-miR-4787-3p, hsa-miR-4787-5p, hsa-miR-483-3p, hsa-miR-483-5p, hsa-miR-484, hsa-miR-485-3p, hsa-miR-485-5p, hsa-miR-487b-3p, hsa-miR-487b-5p, hsa-miR-488-3p, hsa-miR-488-5p, hsa-miR-489-3p, hsa-miR-490-3p, hsa-miR-490-5p, hsa-miR-491-3p, hsa-miR-491-5p, hsa-miR-493-3p, hsa-miR-493-5p, hsa-miR-494-3p, hsa-miR-494-5p, hsa-miR-495-3p, hsa-miR-495-hsa-miR-497-3p, hsa-miR-497-5p, hsa-miR-498, hsa-miR-5001-3p, hsa-miR-5001-5p, hsa-miR-500a-3p, hsa-miR-500a-5p, hsa-miR-5010-3p, hsa-miR-5010-5p, hsa-miR-503-3p, hsa-miR-503-5p, hsa-miR-504-3p, hsa-miR-504-5p, hsa-miR-505-3p, hsa-miR-505-5p, hsa-miR-506-3p, hsa-miR-506-5p, hsa-miR-508-3p, hsa-miR-508-5p, hsa-miR-509-3-5p, hsa-miR-509-3p, hsa-miR-509-5p, hsa-miR-510-3p, hsa-miR-510-5p, hsa-miR-512-5p, hsa-miR-513c-3p, hsa-miR-513c-5p, hsa-miR-514a-3p, hsa-miR-514a-5p, hsa-miR-514b-3p, hsa-miR-514b-5p, hsa-miR-516b-5p, hsa-miR-518c-3p, hsa-miR-518f-3p, hsa-miR-5196-3p, hsa-miR-5196-5p, hsa-miR-519a-3p, hsa-miR-519a-5p, hsa-miR-519c-3p, hsa-miR-519e-3p, hsa-miR-520c-3p, hsa-miR-520f-3p, hsa-miR-520g-3p, hsa-miR-520h, hsa-miR-522-3p, hsa-miR-525-hsa-miR-526b-5p, hsa-miR-532-3p, hsa-miR-532-5p, hsa-miR-539-3p, hsa-miR-539-5p, hsa-miR-542-3p, hsa-miR-542-5p, hsa-miR-543, hsa-miR-545-3p, hsa-miR-545-5p, hsa-miR-548a-3p, hsa-miR-548a-5p, hsa-miR-548ar-3p, hsa-miR-548ar-5p, hsa-miR-548b-3p, hsa-miR-548d-3p, hsa-miR-548d-5p, hsa-miR-548e-3p, hsa-miR-548e-5p, hsa-miR-548h-3p, hsa-miR-548h-5p, hsa-miR-548j-3p, hsa-miR-548j-5p, hsa-miR-548o-3p, hsa-miR-548o-5p, hsa-miR-548v, hsa-miR-551b-3p, hsa-miR-551b-5p, hsa-miR-552-3p, hsa-miR-556-3p, hsa-miR-556-5p, hsa-miR-561-3p, hsa-miR-561-5p, hsa-miR-562, hsa-miR-567, hsa-miR-569, hsa-miR-570-3p, hsa-miR-570-5p, hsa-miR-571, hsa-miR-574-3p, hsa-miR-574-5p, hsa-miR-576-3p, hsa-miR-576-5p, hsa-miR-577, hsa-miR-579-3p, hsa-miR-579-5p, hsa-miR-582-3p, hsa-miR-582-5p, hsa-miR-584-3p, hsa-miR-584-5p, hsa-miR-589-3p, hsa-miR-589-hsa-miR-590-3p, hsa-miR-590-5p, hsa-miR-595, hsa-miR-606, hsa-miR-607, hsa-miR-610, hsa-miR-615-3p, hsa-miR-615-5p, hsa-miR-616-3p, hsa-miR-616-5p, hsa-miR-617, hsa-miR-619-5p, hsa-miR-624-3p, hsa-miR-624-5p, hsa-miR-625-3p, hsa-miR-625-5p, hsa-miR-62′7-3p, hsa-miR-627-5p, hsa-miR-628-3p, hsa-miR-628-5p, hsa-miR-629-3p, hsa-miR-629-hsa-miR-630, hsa-miR-633, hsa-miR-634, hsa-miR-635, hsa-miR-636, hsa-miR-640, hsa-miR-642a-3p, hsa-miR-642a-5p, hsa-miR-643, hsa-miR-645, hsa-miR-648, hsa-miR-6503-3p, hsa-miR-6503-5p, hsa-miR-651-3p, hsa-miR-651-5p, hsa-miR-6511a-3p, hsa-miR-6511a-5p, hsa-miR-652-3p, hsa-miR-652-5p, hsa-miR-653-5p, hsa-miR-654-3p, hsa-miR-654-5p, hsa-miR-657, hsa-miR-659-3p, hsa-miR-660-3p, hsa-miR-660-5p, hsa-miR-664b-3p, hsa-miR-664b-5p, hsa-miR-671-3p, hsa-miR-671-5p, hsa-miR-675-3p, hsa-miR-675-5p, hsa-miR-7-1-3p, hsa-miR-7-5p, hsa-miR-708-3p, hsa-miR-708-5p, hsa-miR-744-3p, hsa-miR-744-5p, hsa-miR-758-3p, hsa-miR-758-5p, hsa-miR-765, hsa-miR-766-3p, hsa-miR-766-5p, hsa-miR-767-3p, hsa-miR-767-5p, hsa-miR-769-3p, hsa-miR-769-5p, hsa-miR-802, hsa-miR-873-3p, hsa-miR-873-5p, hsa-miR-874-3p, hsa-miR-874-5p, hsa-miR-876-3p, hsa-miR-8′76-5p, hsa-miR-885-3p, hsa-miR-885-5p, hsa-miR-887-3p, hsa-miR-887-5p, hsa-miR-9-3p, hsa-miR-9-5p, hsa-miR-92a-1-5p, hsa-miR-92a-2-5p, hsa-miR-92a-3p, hsa-miR-92b-3p, hsa-miR-92b-5p, hsa-miR-93-3p, hsa-miR-93-5p, hsa-miR-941, hsa-miR-942-3p, hsa-miR-942-hsa-miR-96-3p, hsa-miR-96-5p, hsa-miR-98-3p, hsa-miR-98-5p, hsa-miR-99a-3p, hsa-miR-99a-5p, hsa-miR-99b-3p, and hsa-miR-99b-5p.

In some embodiments, the cell state classifier of the present disclosure is used in a bacterial cell. Though naturally-occurring bacterial cells lack true miRNAs (e.g., as described in Tjaden et al., Nucleic Acids Res. 34 (9): 2791-802), short non-coding RNA sequences have been identified in bacterial genome that broadly have comparable function as eukaryotic miRNAs. Such bacterial short non-coding RNAs function similarly as the miRNAs of the present disclosure and may be detected by the cell state classifier described herein.

For classifying a cell type (e.g., a cancer cell), one skilled in the art is familiar with the microRNAs that express specifically in such cell type but not in other cell types, and their respective nucleotide sequences. One skilled in the art is also familiar with the designing the target sites for the microRNA to be detected. Non-limiting, exemplary microRNA and respective target site sequences are provided in Table 5.

TABLE 5
Exemplary Synthetic microRNA and target sites

| microRNA | Nucleotide Sequence | |
|---|---|---|
| Name | Encoding microRNA | Target Sequence |
| FF3 | TTTGTATTCAGCCCATATCG | AACGATATGGGCTGAATACAAA |
| | (SEQ ID NO: 59) | (SEQ ID NO: 64) |
| FF4 | TTTAATTAAAGACTTCAAGCG | CCGCTTGAAGTCTTTAATTAAA |
| | (SEQ ID NO: 60) | (SEQ ID NO: 65) |
| FF5 | TAATTGTCAAATCAGAGTGC | AAGCACTCTGATTTGACAATTA |
| | (SEQ ID NO: 61) | (SEQ ID NO: 66) |
| FF6 | TTTATGAGGAATCTCTTTGG | AACCAAAGAGATTCCTCATAAA |
| | (SEQ ID NO: 62) | (SEQ ID NO: 67) |
| T1 | TTCGAAGTATTCCGCGTACG | CACGTACGCGGAATACTTCGAA |
| | (SEQ ID NO: 63) | (SEQ ID NO: 68) |

One or more (e.g., 1, 2, 3, 4, 5, 6, 7, 8, 9, 10, or more) target sites of the microRNAs to be detected by the cell state classifier are placed in each circuit (e.g., first or second sensor circuit, signal circuit, etc.) in a non-coding region, e.g., upstream and/or downstream of the nucleotide sequence encoding the protein that is produced by the circuit (e.g., activator, repressor, or output molecule). Being “upstream” means the microRNA target sites are placed 5′ of the nucleotide sequence encoding the protein that is produced by the circuit (e.g., activator, repressor, or output molecule). Being “downstream” means the microRNA target sites are placed 3′ of the nucleotide sequence encoding the protein that is produced by the circuit (e.g., activator, repressor, or output molecule).

In some embodiments, one or more (e.g., 1, 2, 3, 4, 5, 6, 7, 8, 9, 10, or more) microRNA target sites are placed upstream of and is immediately adjacent to (no nucleotides in between) the nucleotide sequence encoding the protein that is produced by the circuit (e.g., activator or output molecule). In some embodiments, one or more (e.g., 1, 2, 3, 4, 5, 6, 7, 8, 9, 10, or more) microRNA target sites are placed upstream of and is separated by a nucleotide spacer from the nucleotide sequence encoding the protein that is produced by the circuit (e.g., activator, kinase, phosphatase, or output molecule). In some embodiments, the nucleotide spacer is 1-20 nucleotides long. For example, the nucleotide spacer may be 1, 2, 3, 4, 5, 6, 7, 8, 9, 10, 11, 12, 13, 14, 15, 16, 17, 18, 19, or 20 nucleotides long. Nucleotide spacers longer than 20 nucleotide may also be used.

In some embodiments, one or more (e.g., 1, 2, 3, 4, 5, 6, 7, 8, 9, 10, or more) microRNA target sites are placed downstream of and is immediately adjacent to (no nucleotides in between) the nucleotide sequence encoding the protein that is produced by the circuit (e.g., activator, kinase, phosphatase, or output molecule). In some embodiments, one or more (e.g., 1, 2, 3, 4, 5, 6, 7, 8, 9, 10, or more) microRNA target sites are placed downstream of and is separated by a nucleotide spacer from the nucleotide sequence encoding the protein that is produced by the circuit (e.g., activator, kinase, phosphatase, or output molecule). In some embodiments, the nucleotide spacer is 1-20 nucleotides long. For example, the nucleotide spacer may be 1, 2, 3, 4, 5, 6, 7, 8, 9, 10, 11, 12, 13, 14, 15, 16, 17, 18, 19, or 20 nucleotides long. Nucleotide spacers longer than 20 nucleotide may also be used.

In some embodiments, one or more (e.g., 1, 2, 3, 4, 5, 6, 7, 8, 9, 10, or more) microRNA target sites are placed upstream and/or downstream of and is immediately adjacent (no nucleotides in between) to the nucleotide sequence encoding the protein that is produced by the circuit (e.g., activator, kinase, phosphatase, or output molecule). In some embodiments, one or more (e.g., 1, 2, 3, 4, 5, 6, 7, 8, 9, 10, or more) microRNA target sites are placed upstream and/or downstream of and is separated by a nucleotide spacer from the nucleotide sequence encoding the protein that is produced by the circuit (e.g., activator, kinase, phosphatase, or output molecule). In some embodiments, the nucleotide spacer is 1-20 nucleotides long. For example, the nucleotide spacer may be 1, 2, 3, 4, 5, 6, 7, 8, 9, 10, 11, 12, 13, 14, 15, 16, 17, 18, 19, or 20 nucleotides long. Nucleotide spacers longer than 20 nucleotide may also be used. In some embodiments, placing multiple microRNA target sites at different locations of each circuit strengthens (e.g., by at least 30%) the inhibitory effect of the microRNA on the product of the circuit. When multiple microRNA target sites are used, there may be a nucleotide spacer (e.g., a nucleotide spacer of 1, 2, 3, 4, 5, 6, 7, 8, 9, or 10 nucleotides long), or no space between each target site.

### Feedback Controller Circuit and Cell State Classifier Functionality

Some aspects of the present disclosure provide the functionality of the feedback controller circuits provided herein and methods of using them. In some embodiments, the methods comprise delivering a feedback controller circuit described herein into a cell in vitro, ex vivo or in vivo (e.g., by any of the methods described herein and known to one skilled in the art). In some embodiments, the methods comprise maintaining the cell containing a feedback controller circuit, such as by culturing a cell in vitro or ex vivo, or by maintaining the viability of the cell in vivo. In some embodiments, the maintaining is carried out under conditions to allow the feedback controller circuit to function. In some embodiments, the presence of the cell state classifier in the cell does not change the native microRNA profile of the cell. In some embodiments, the presence of the cell state classifier in the cell does not after expression of genes from chromosomes of the cell.

Some aspects of the present disclosure provide the functionality of the cell state classifiers and methods of using them. In some embodiments, the methods comprise delivering the cell state classifier described herein into a cell in vitro, ex vivo or in vivo (e.g., by any of the methods described herein and known to one skilled in the art). In some embodiments, the methods comprise maintaining the cell containing the cell state classifier, such as by culturing a cell in vitro or ex vivo, or by maintaining the viability of the cell in vivo. In some embodiments, the maintaining is carried out under conditions to allow the cell state classifier to function. In some embodiments, the presence of the cell state classifier in the cell does not change the native microRNA profile of the cell.

Once introduced to a cell that has a microRNA profile, the cell state classifier described herein is able to detect the microRNAs in the cell and produce an output (e.g., a detectable molecule or a therapeutic molecule) accordingly. In some embodiments, no microRNA input is detected, for example, if none of the microRNAs the cell state classifier designed to detected (either microRNA-high or microRNA-low) expresses (e.g., expression level is not detectable). As such, the activator and the kinase of the first sensor circuit are expressed in the absence of microRNA-low inhibition, leading to phosphorylation of the activator. Further, in the absence of the microRNA-high, the phosphatase expresses, dephosphorylating the activator. Thus, a competition exists between phosphorylating and dephosphorylating the activator. It is to be understood that in such situations, the phosphatase dominates, and the activator remains unphosphorylated. The unphosphorylated activator does not bind or activate the activatable promoter in the signal circuit, leading to no production of the output molecule.

In some embodiments, the first microRNA (microRNA-low) expresses (e.g., has a detectable expression level by the cell state classifier), and the activator and the kinase in the first sensor circuit does not express because the first microRNA mediate the degradation of the mRNA encoding the activator and the kinase (translational control). As such, the activatable promoter of the signal circuit is not activated, leading to no expression of the first output molecule.

In contrast, in some embodiments, the first microRNA (microRNA-low) does not express (e.g., has a detectable expression level by the cell state classifier), and the activator and the kinase in the first sensor circuit express. The kinase phosphorylates the activator, causing a conformational change in the activator that allows it to bind the activatable promoter of the signal circuit, activating the expression of the output molecule.

In some embodiments, the second microRNA (microRNA-high) expresses (e.g., has a detectable expression level by the cell state classifier), and phosphatase does not express, because the second microRNA mediates the degradation of the mRNA encoding the phosphatase (translational control). As a result, the activator remains phosphorylated and activates the expression of the output molecule.

In some embodiments, both the first microRNA (microRNA-low) and the second microRNA (microRNA-high) express, repressing the expression of the activator, the kinase, and the phosphatase (e.g., via microRNA binding sites in the first and second sensor circuits). As a result, the activatable promoter in the signal circuit remains inactive due to the lack of the phosphorylated activator, leading to no expression of the output molecule.

As such, the cell state classifier also has a logic function, where the cell state classifier produces an output molecule only when a matching microRNA profile is detected. A matching microRNA profile means the first microRNA (microRNA-low) does not express (e.g., undetectable by the cell state classifier), and the second microRNA (microRNA-high) expresses (e.g., at least detectable by the cell state classifier), and the output molecule is produced. In some embodiments, the first microRNA (microRNA low) expresses (e.g., expression level is detectable by the cell state classifier) or has high expression level, and the second microRNA (microRNA-high) does not express (e.g., expression level is not detectable by the cell state classifier), and no output molecule is produced by the cell state classifier. In some embodiments, the first microRNAs (microRNA low) expresses (e.g., expression level is detectable by the cell state classifier), and the second microRNA (microRNA-high) does not express (e.g., expression level is not detectable by the cell state classifier), and no output molecule or very low output molecule is produced by the cell state classifier. In some embodiments, the first microRNA (microRNA low) expresses (e.g., expression level is detectable by the cell state classifier) or has high expression level, and the second microRNA expresses or has high expression level, and no output molecule or very low output molecule is produced by the cell state classifier.

By placing the target sites for the first or second microRNA (microRNA-low or microRNA-high) in different circuits of the cell state classifier, additional functions of the cell state classifiers can be provided. For example, if the first sensor circuit comprises target sites for the second microRNA (miRNA-high) and the second sensor circuit comprising target sites for the first microRNA (miRNA-low), then the output of the cell state classifier circuit would be reversed such that output would be produced only in cells in which miRNA-low, but not miRNA-high, is expressed.

In some embodiments, to classify the cell, the method further comprises detecting an output molecule produced by the cell state classifier. For example, the output molecule may be fluorescent protein or an enzyme that acts on a substrate. One skilled in the art is familiar with methods of detecting different detectable molecules.

### Applications

The feedback controller circuits and cell state classifiers described herein may be used for a variety of applications. In some embodiments, the feedback controller circuit or cell state classifier is used for diagnostic purposes. For example, in some embodiments, the cell state classifier may be designed to detect the microRNA profile in a diseased cell (e.g., a cancer cell). As such, if an output signal is detected when such cell state classifier is delivered to a cell, the cell may be classified as a diseased cell (e.g., a cancer cell). For diagnostic purposes, the output molecules of the feedback controller circuit or cell state classifier is typically a detectable molecule (e.g., a fluorescent protein or chemiluminescent protein). Depending on the cell type to be detected and the specific microRNA profile, in some embodiments, the expression of an output molecule indicates a diseased cell. In some embodiments, lack of expression of the output molecule indicates a diseased cell.

In another example, the feedback controller circuit or cell state classifier is used for therapeutic purposes. For example, in some embodiments, the cell state classifier is designed to detect the microRNA profile in a diseased cell (e.g., a cancer cell) and to produce an output molecule that is a therapeutic molecule (e.g., a therapeutic protein or RNA). Upon detecting of a matching microRNA profile in the diseased cell, the cell state classifier produces the therapeutic molecule, thus treating the disease. Such therapeutic methods are highly specific to the diseased cell and have low impact on healthy cells because the cell state classifier will not detect a matching microRNA profile in a healthy and thus will not produce the output molecule. Further, the therapeutic effect of the cell state classifier is long-lasting. For example, the cell state classifier will continue to produce the therapeutic molecule until the diseased cell no longer has a matching microRNA profile that fit the disease (e.g., cancer). Additionally, in some embodiments, the feedback controller circuit maintains expression of the output molecule at a consistent level even if transcriptional resources are diverted to other genetic loci. Once therapeutic effects have taken place, the cell state classifier can sense the change in the microRNA profile (e.g., from cancer profile to normal profile) and stop the production of the therapeutic molecule.

In some embodiments, a feedback controller circuit maintains output molecule expression at a consistent level, with co-expression of the phosphatase regulator preventing excess output molecule expression, and the extent of phosphatase regulator activity being tunable by controlling the concentration of a stabilizing small molecule. Thus, a kinase may be expressed to continually phosphorylate an activator, such that activation leading to output molecule expression is continuously promoted, while phosphatase activity that inhibits transcription may be tuned by withholding the stabilizing small molecule and allowing the phosphatase to degrade, such that output molecule expression can be manipulated more quickly than in cell state classifiers that use only miRNA-based translational regulation to modulate output molecule expression.

For either diagnostic or treatment purposes, the cell may be in vitro (e.g., cultured cell), ex vivo (e.g., isolated from a subject), or in vivo in a subject. For in vivo applications, in some embodiments, the method comprises administering an effective amount of a composition comprising the feedback controller circuit or cell state classifier described herein to a subject in need thereof.

The composition can further comprise additional agents (e.g. for specific delivery, increasing half-life, or other therapeutic agents). In some embodiments, the composition further comprises a pharmaceutically acceptable excipient. The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” is a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Some examples of materials which can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as peptides and amino acids (23) serum component, such as serum albumin, HDL and LDL; (24) C2-C12 alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier” or the like are used interchangeably herein.

An “effective amount” refers to the amount of the feedback controller circuit, cell state classifier, or composition comprising such required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a disorder. Alternatively, sustained continuous release formulations of agent may be appropriate. Various formulations and devices for achieving sustained release are known in the art.

An effective amount of the feedback controller circuit or cell state classifier or composition comprising such may be administered repeatedly to a subject (e.g., 2, 3, 4, 5, 6, 7, 8, 9, 10 times or more). In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the agents used) can vary over time.

In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the agent (such as the half-life of the agent, and other considerations well known in the art). For the purpose of the present disclosure, the appropriate dosage of the feedback controller circuit or cell state classifiers compositions as described herein will depend on the specific agent (or compositions thereof) employed, the formulation and route of administration, the type and severity of the disorder, previous therapy, the subject's clinical history and response to the agents, and the discretion of the attending physician. Typically the clinician will administer an agent until a dosage is reached that achieves the desired result.

Administration can be continuous or intermittent, depending, for example, upon the recipient's physiological condition, and other factors known to skilled practitioners. The administration of an agent may be essentially continuous over a preselected period of time or may be in a series of spaced dose, e.g., either before, during, or after developing a disorder.

A “subject” refers to human and non-human animals, such as apes, monkeys, horses, cattle, sheep, goats, dogs, cats, rabbits, guinea pigs, rats, and mice. In one embodiment, the subject is human. In some embodiments, the subject is an experimental animal or animal substitute as a disease model. A “subject in need thereof” refers to a subject who has or is at risk of a disease or disorder (e.g., cancer).

The feedback controller circuits or cell state classifiers of the present disclosure may be delivered to a subject (e.g., a mammalian subject, such as a human subject) by any in vivo delivery method known in the art. For example, engineered nucleic acids may be delivered intravenously. In some embodiments, engineered nucleic acids are delivered in a delivery vehicle (e.g., non-liposomal nanoparticle or liposome). In some embodiments, the feedback controller circuits or cell state classifiers are delivered systemically to a subject having a cancer or other disease and produces a therapeutic molecule specifically in cancer cells or diseased cells of the subject. In some embodiments, the feedback controller circuits or cell state classifiers are delivered to a site of the disease or disorder (e.g., site of cancer).

Non-limiting examples of cancers that may be treated using the feedback controller circuits or cell state classifiers and methods described herein include: premalignant neoplasms, malignant tumors, metastases, or any disease or disorder characterized by uncontrolled cell growth such that it would be considered cancerous or precancerous. The cancer may be a primary or metastatic cancer. Cancers include, but are not limited to, ocular cancer, biliary tract cancer, bladder cancer, pleura cancer, stomach cancer, ovary cancer, meninges cancer, kidney cancer, brain cancer including glioblastomas and medulloblastomas, breast cancer, cervical cancer, choriocarcinoma, colon cancer, endometrial cancer, esophageal cancer, gastric cancer, hematological neoplasms including acute lymphocytic and myelogenous leukemia, multiple myeloma, AIDS-associated leukemias and adult T-cell leukemia lymphoma, intraepithelial neoplasms including Bowen's disease and Paget's disease, liver cancer, lung cancer, lymphomas including Hodgkin's disease and lymphocytic lymphomas, neuroblastomas, oral cancer including squamous cell carcinoma, ovarian cancer including those arising from epithelial cells, stromal cells, germ cells and mesenchymal cells, pancreatic cancer, prostate cancer, rectal cancer, sarcomas including leiomyosarcoma, rhabdomyosarcoma, liposarcoma, fibrosarcoma, and osteosarcoma, skin cancer including melanoma, Kaposi's sarcoma, basocellular cancer, and squamous cell cancer, testicular cancer including germinal tumors such as seminoma, non-seminoma, teratomas, choriocarcinomas, stromal tumors and germ cell tumors, thyroid cancer including thyroid adenocarcinoma and medullar carcinoma, and renal cancer including adenocarcinoma and

Wilms' tumor. Commonly encountered cancers include breast, prostate, lung, ovarian, colorectal, and brain cancer. In some embodiments, the tumor is a melanoma, carcinoma, sarcoma, or lymphoma.

### EXAMPLES

### Example 1: Robust and Tunable Signal Processing in Mammalian Cells Via Engineered Covalent Modification Cycles

Rewired and synthetic signaling networks can impart cells with new functionalities and enable efforts in engineering cell therapies and directing cell development. However, there is a need for tools to build synthetic signaling networks that are tunable, can precisely regulate target gene expression, and are robust to perturbations within the complex context of mammalian cells. Here, proteins derived from bacterial two-component signaling pathways were used to develop synthetic phosphorylation-based and feedback-controlled devices in mammalian cells with such properties. First, kinase and phosphatase proteins from the bifunctional histidine kinase EnvZ were isolated. These proteins were then used to engineer a synthetic covalent modification cycle, in which the kinase and phosphatase competitively regulate phosphorylation of the cognate response regulator OmpR, enabling analog tuning of OmpR-driven gene expression. Further, the phosphorylation cycle was extended by linking phosphatase expression to small molecule and miRNA inputs in the cell, with the latter enabling cell-type specific signaling responses and accurate cell type classification. Finally, a tunable, negative feedback controller was implemented by co-expressing the kinase-driven output gene with the small molecule-tunable phosphatase regulator. This negative feedback substantially reduced cell-to-cell noise in output molecule expression and mitigated the effects of cellular perturbations due to off-target regulation and resource competition. The research described in this Example therefore establishes tunable, precise, and robust control over cell behavior with synthetic signaling networks. The tunable phosphorylation-based feedback controller circuits described in this Example are also described in Jones et al. bioRxiv. 2021. doi: 10.1101/2021.03.30.437779, first posted online Jul. 19, 2021, which is incorporated by reference herein in its entirety.

Synthetic control of protein phosphorylation enabled programming of sophisticated cellular behaviors via engineered signaling networks. Towards this goal, synthetic phosphoregulated genetic circuits for tuning of gene expression in mammalian cells were constructed using proteins derived from bacterial two component signaling (TCS). In most natural TCS systems, environmental cues cause membrane-bound histidine kinases (HKs) to autophosphorylate on a conserved histidine residue, then transfer the phosphate to an aspartate residue on their cognate response regulator (RR) protein. When phosphorylated, RRs typically bind to DNA and regulate transcription. Since TCS is absent and histidine/aspartate phosphorylation is rare in animals, TCS is naturally orthogonal to nearly all mammalian signaling pathways, enabling insulation of engineered phosphoregulated genetic circuits from interference by endogenous phosphorylation and dephosphorylation processes. Recent efforts have developed synthetic signal transduction systems in mammalian cells by fusing HKs to ligand-dimerizable proteins. However, to impart tunability and robustness into such devices, reversable control of RR phosphorylation is needed. Most HKs are bifunctional, dephosphorylating their cognate RR in the absence of signal input, presenting a challenge to using HK—RR pairings to modulate gene expression to a desired level.

To generate monofunctional kinases and phosphatases and improve the tunability of gene expression, various mutations, truncations, and domain rearrangements of the well-studied HK EnvZ were screened. Using an engineered kinase (EnvZK) and phosphatase (EnvZP), synthetic futile cycles were constructed, wherein the enzymes compete for the addition or subtraction of phosphate to EnvZ's cognate RR, OmpR. By adjusting EnvZ kinase activity via titration of its DNA dosage, and modulating EnvZ phosphatase activity by regulating amount with DNA dosage titration, regulating translation with cell type-specific miRNAs, and regulating phosphatase stability with degradation domains and small molecule stabilization, highly tunable genetic circuits were generated.

To impart robustness to perturbations, negative feedback control was implemented via co-expression of EnvZP with the OmpR-driven output. This controller significantly reduces sensitivity to undesirable transcriptional or translational regulation via either resource loading or simulated off-target miRNA targeting. This work provides a foundation for constructing precisely tunable and robust synthetic signaling networks in mammalian cells, with applications in programming cell therapy and directing cellular development.

### Design

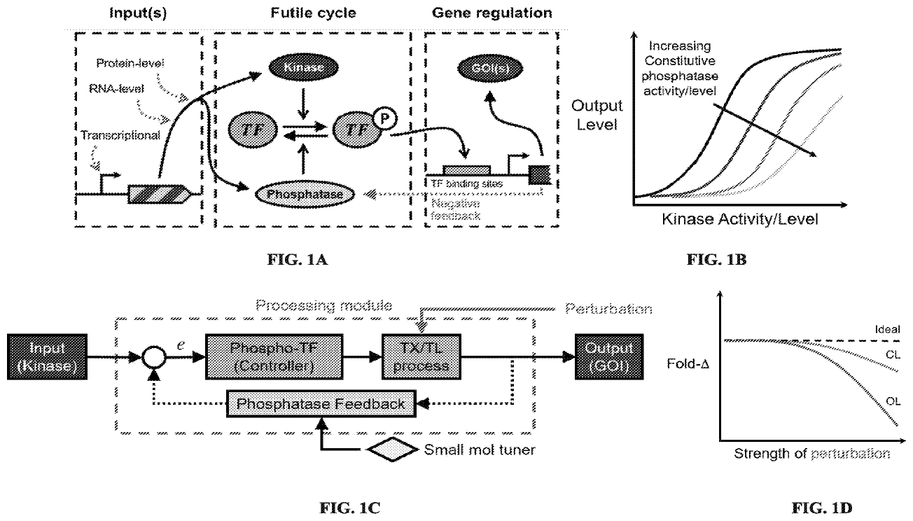

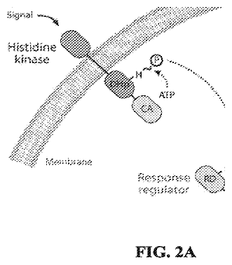

As shown in FIGS. 1A-1D, circuits of the present disclosure use a kinase as an input and express both a phosphatase and gene of interest (GOI) as output. The kinase phosphorylates a transcription factor (TF), enabling it to bind to DNA and activate transcription (TX) and translation (TL) of the output GOI. These TX/TL events comprise the “process” to be regulated by the controller. Perturbations from outside of the circuit affect the TX/TL process. Negative feedback is implemented by co-expressing a phosphatase regulator with the output protein. The phosphatase dephosphorylates the TF, thereby reducing its own expression level. The relative expression level of the phosphatase compared to the output can be tuned with a small molecule. The “controller” is formed by the competing phosphorylation and dephosphorylation of the TF by the kinase and phosphatase. When the activity of the kinase is higher than that of the phosphatase, the amount of phosphorylated TF increases, thus increasing GOI and phosphatase production. When the activity of the phosphatase is higher, the reverse occurs, and GOI and phosphatase activity are reduced.

### Selection of HK-RR Proteins

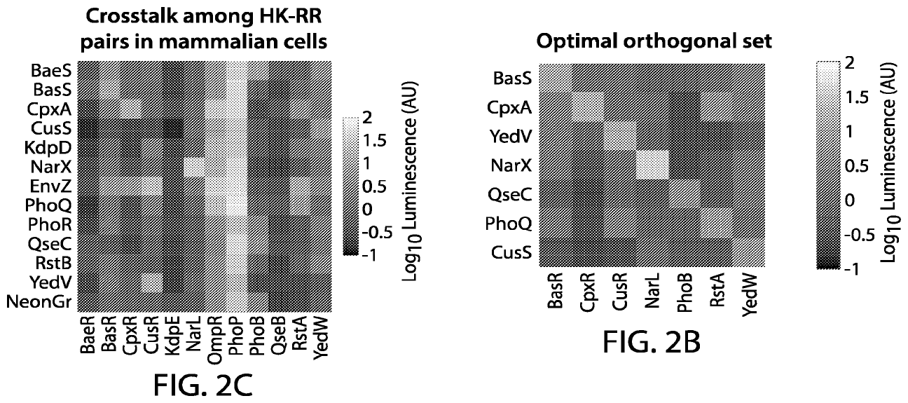

Kinase and phosphatase proteins have been derived from bacterial two component signaling (TCS) for use in mammalian cells. TCS systems comprise pairs of transmembrane histidine kinases (HKs) and cytoplasmic response regulators (RRs). Typically, upon extracellular signal induction, the HK will auto-phosphorylate a conserved histidine residue in the dimerization and histidine phosphorylation (DHp) domain of the protein. The phosphate group is then rapidly transferred to the HK's cognate RR protein on a conserved aspartate residue in the receiver domain of the protein (FIG. 2A). This phosphate group causes a conformational change in the RR, allowing the phosphorylated form to mediate an intracellular process in response to the external signal. Many RRs are TFs, and their binding to DNA is controlled by this conformational change. Several HK/RR pairs that function orthogonally in mammalian cells have been identified (FIG. 2B). Each HK in FIG. 2A is specific to a cognate RR, allowing multiple pairings to be used in the same cell for separately tunable regulation of multiple outputs. Additionally, other RRs can be phosphorylated by multiple HKs, allowing feedback controllers using by those general RRs to activate multiple distinct genetic circuits (FIG. 2C).

### Improved Kinase and Phosphatase Design

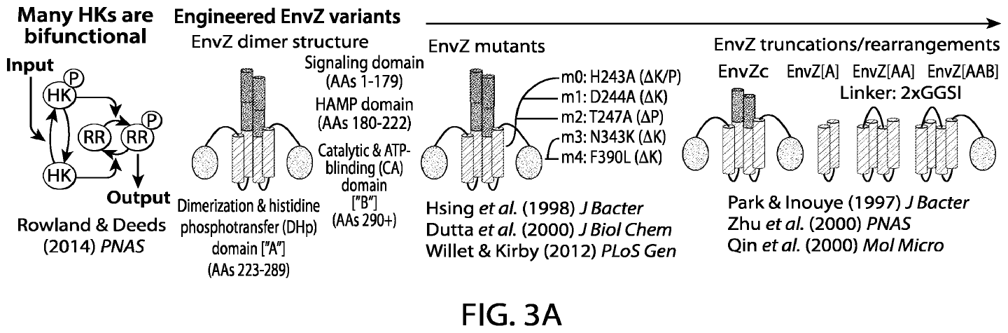

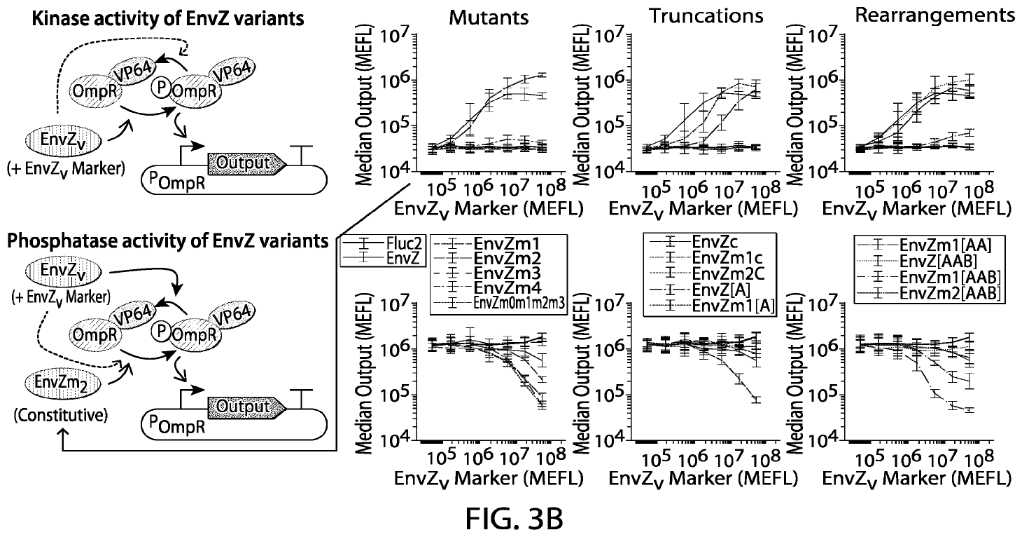

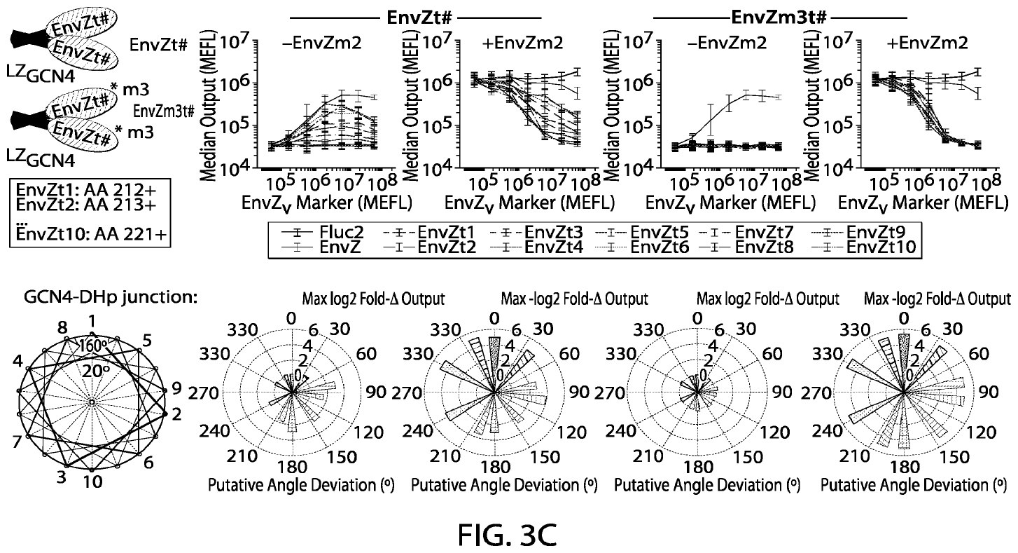

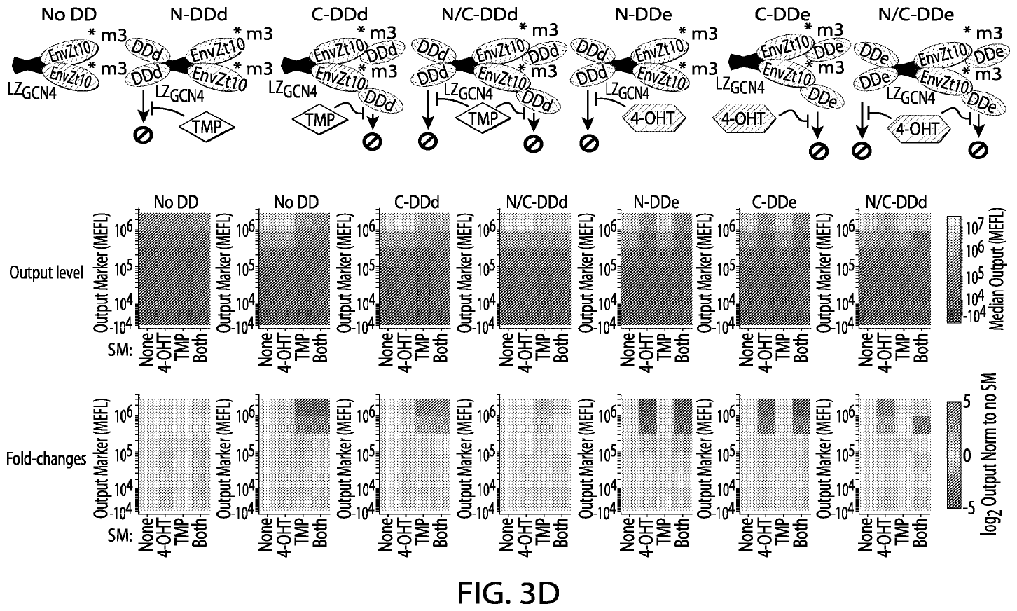

The well-studied EnvZ-OmpR system was used to develop feedback controllers of the present disclosure, using separate kinase and phosphatase variants of EnvZ, and an OmpR-VP64 fusion protein as an activator for promoting transcription of a reporter protein. Kinase and phosphatase variants of the HK EnvZ through mutation and/or truncation (FIG. 3A). EnvZm2, containing a T247A mutation, was particularly effective in phosphorylating OmpR (FIG. 3B). However, the phosphatase EnvZ activity was weak compared to the EnvZm2 kinase. To create a stronger phosphatase, a DHp domain of EnvZ was fused to a leucine zipper GCN4 domain, with GCN4 at the N-terminus and EnvZm2 at the C-terminus, to lock the DHp domain of an HK into fixed conformations (FIG. 3C). To abrogate kinase activity, a N343K (m3) mutation was introduced into the GCN4-EnvZ fusion protein, which reduced its ability to bind ATP and thus phosphorylate OmpR, without adversely affecting its phosphatase activity. Finally, the length of the EnvZm3 DHp domain in the fusion protein was varied. It was determined that the angle at which the GCN4 domain joined the EnvZm2m3 DHp domain strongly influenced the phosphatase activity, with the variant containing only amino acids starting from amino acid 221 of EnvZ having optimal phosphatase activity. This strategy created a robust EnvZ phosphatase for use in a feedback controller.

### Tuning of Phosphatase Stability

The phosphatase regulator was further modified to allow tunability by use of a small molecule. In a genetic circuit using a feedback controller, the strength of feedback is proportional to the level of phosphatase produced compared to the output GOI. Expression of both proteins from the same nucleic acid and translation from the same mRNA, such as by separating coding sequences by a 2A motif or open reading frames IRES cassette, ensures maximal coupling of transcription and translation of the two proteins. Such coupling would ensure that changes in output levels due to perturbations in TX/TL are faithfully captured by the phosphatase and fed back into the TF phosphorylation cycle. However, modulating phosphatase activity, and thus the strength of feedback, allows further tuning of output gene expression that may allow output gene expression to respond to changes in cellular conditions, if desired. To independently tune the phosphatase level separately from the GOI, one or more small molecule-inducible degradation domains were fused to the GCN4-EnvZm3t10 fusion proteins described in FIG. 3C. Different fusions using DDd (stabilized by TMP) and DDe (stabilized by 4-OHT) degradation domains revealed that N-terminal fusions of either domain yielded the best dynamic range of phosphatase activity in response to small molecule stabilization (FIG. 3D).

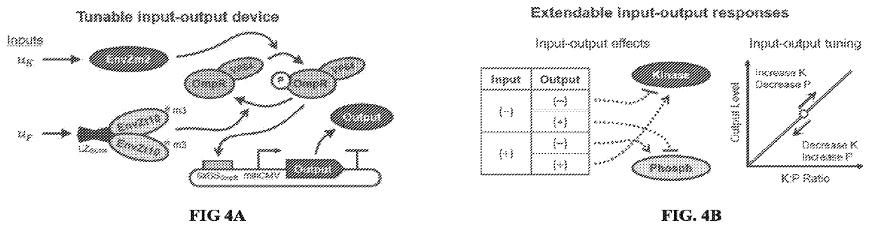

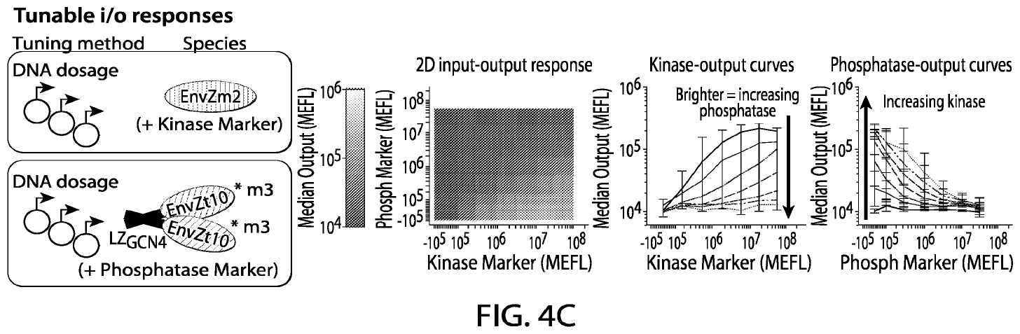

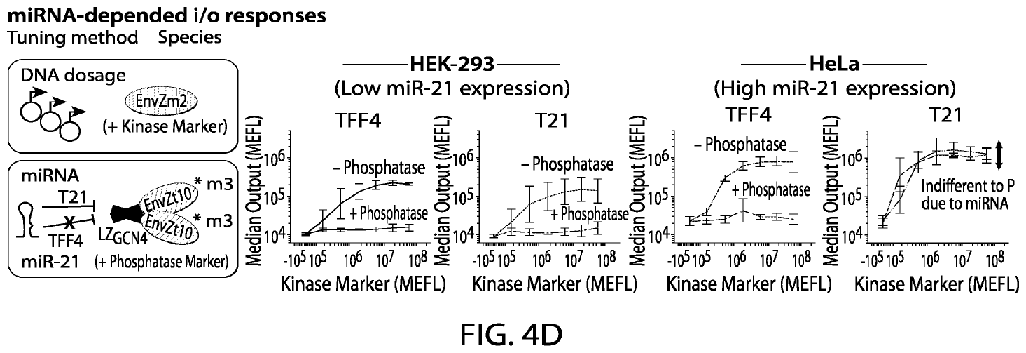

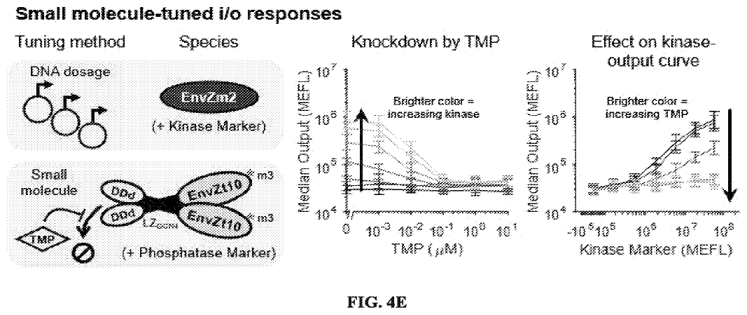

Alternative means of tuning output gene expression were tested using a tunable input-output circuit in which kinase and phosphatase were expressed independently to modulate OmpR-VP64 phosphorylation, which controlled expression of an output gene (FIG. 4A).

Kinase activity was tuned by titrating the DNA dosage of the vector encoding the EnvZm2 kinase. Phosphatase activity was tuned by modulating the amount of constitutively produced phosphatase regulator via titration of DNA dosage (FIG. 4C), modulating translation of the phosphatase regulator by incorporating miRNA target sites into the RNA encoding it (FIG. 4D), or modulating phosphatase regulator stability by incorporating DDd at the N-terminus and titrating the concentration of TMP (FIG. 4E). In DNA dosage titration experiments, increasing the amount of kinase or phosphatase rendered output gene expression less sensitive to changes in the other component (e.g., when high amounts phosphatase DNA were introduced, increasing the amount of kinase DNA had a marginal effect on output gene expression) (FIG. 4C). Incorporating miRNA target sites into the RNA encoding the phosphatase regulator allowed efficient knockdown of phosphatase regulator expression in cells expressing the cognate miRNA, such as HeLa cells in which miR-21 is abundant, but not in cells that do not express the cognate miRNA, such as HEK-293 cells where little miR-21 is present (FIG. 4D). Thus, expression of the phosphatase regulator can be selectively expressed based on the miRNA profile in a cell. In experiments using the phosphatase regulator containing DDd, the stability of the phosphatase regulator was varied by titrating the concentration of TMP, which stabilizes the DDd and prevents degradation of the phosphatase regulator. These experiments revealed that when TMP is absent, output gene expression is sensitive to increases in kinase activity (FIG. 4E). However, as the TMP concentration increased, output gene expression became less sensitive to changes in kinase abundance. These results indicate that incorporation of a degradation domain allows tuning of phosphatase activity, such that the regulator can be allowed to degrade when responsiveness to changes in cellular conditions is desired, or stabilized to decrease responsiveness when robustness is desired.

### Tunable Feedback Controller Implementation

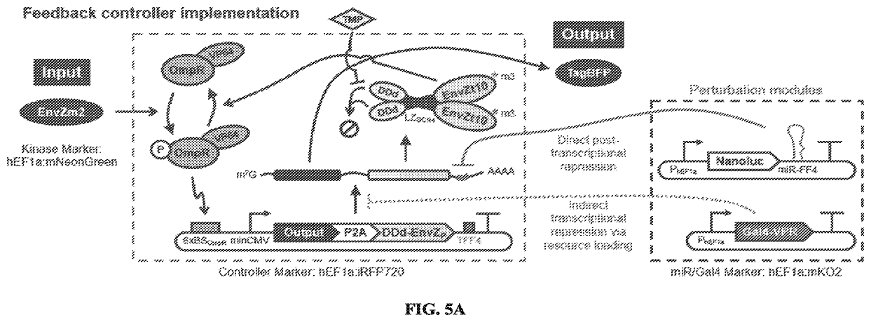

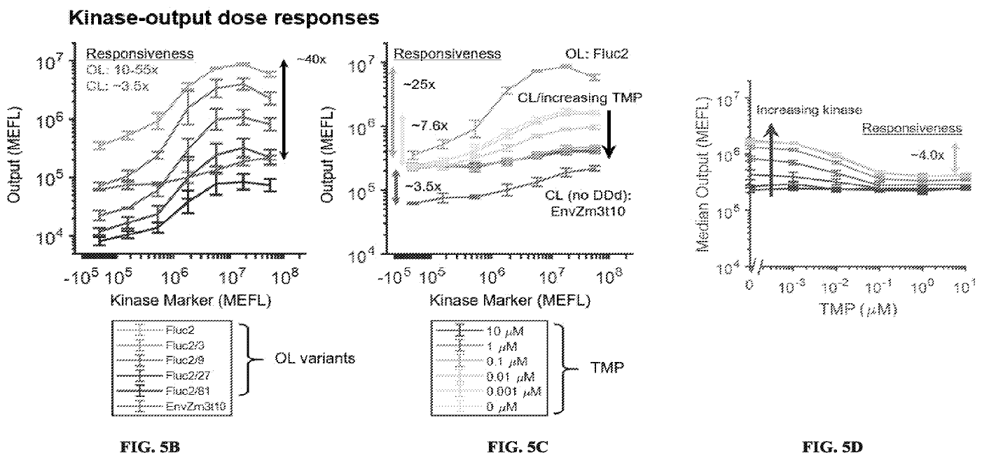

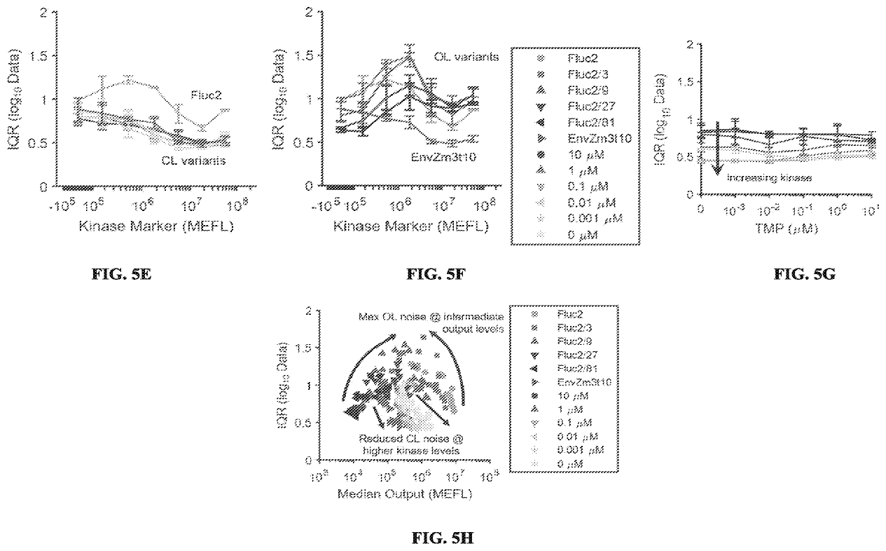

A small molecule-tuned phosphorylation-based feedback controller depicted in FIG. 5A was produced by combining the regulatory features shown in the preceding paragraphs. A phosphatase regulator comprising, in N-to-C-terminal order, a DDd degradation domain, a GCN4 leucine zipper domain, and a EnvZm3t10 phosphatase domain, is co-expressed with the output GOI from the same mRNA transcript via 2A-linker. The relative expression of the output and phosphatase regulator (and thus feedback strength) is tuned by TMP, with higher concentrations stabilizing the phosphatase regulator and increasing the amount of phosphatase activity. The input is a kinase+/phosphatase− mutant of EnvZ (EnvZm2). The circuit is referred to as “closed loop” (CL) when it includes the phosphatase, and “open loop” (OL) without it. OL circuits were sensitive to changes in kinase activity, with output gene expression varying by up to 55× between the lowest and highest amounts of kinase expression (FIG. 5B). By contrast, CL circuits containing the EnvZm3t10 phosphatase regulator varied by only 3.5× between the highest and lowest amounts of kinase expression, indicating that the phosphatase regulator rendered the circuit robust to changes in kinase expression. When the DDd domain was incorporated, CL circuit responsiveness was increased slightly if TMP was absent, with variation of 7.5× between the highest and lowest kinase expression levels (FIG. 5C). However, increasing concentrations of TMP stabilized the phosphatase regulator, such that responsiveness at TMP concentrations of 10 μM was similar to that of circuits containing an EnvZm3t10 phosphatase regulator that lacked a degradation domain (FIGS. 5C-5D). The variability output expression from each genetic circuit, in terms of the interquartile range of expression levels, was also analyzed. Variability was markedly lower in CL circuits than in OL circuits lacking a phosphatase regulator, with minimal sensitivity to TMP concentrations (FIGS. 5E-5H). Surprisingly, variability of expression from CL circuits was reduced at higher kinase levels.

### Robustness to Perturbations

To determine the robustness of feedback controllers to perturbations in intracellular conditions, miR-FF4 or nucleic acids encoding Ga14-VPR were introduced into cells. Ga14-VPR is a potent transcriptional activator, and its expression in the cell sequesters transcriptional resources, which indirectly inhibits transcription of the output molecule from the signal circuit via the squelching effect. miR-FF4 targets the mRNA encoding the output molecule and phosphatase regulator, marking the mRNA for degradation and preventing translation of both encoded proteins.

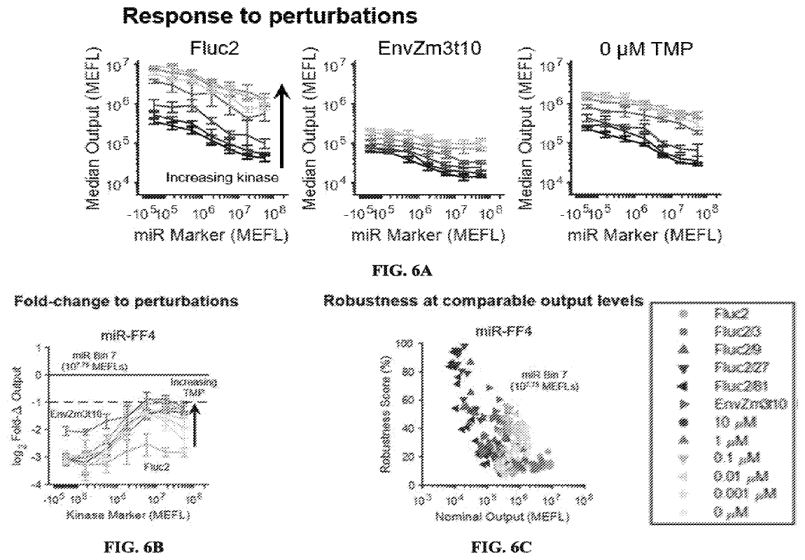

To test the ability of the feedback controller to protect output gene expression from off-target regulation, HEK-293FT cells were co-transfected with CL or OL circuits, varying the dosage of kinase-encoding DNA, TMP (if CL circuits encoded a phosphatase regulator with a DDd domain), and miR-FF4. In these experiments, the mRNA encoding the output molecule and phosphatase regulator contained an miR-FF4 target site in the 3′UTR, allowing transfected miR-FF4 to off-target translational regulation by an endogenous miRNA. Output expression of the CL circuits was markedly less sensitive to changes in miR-FF4 abundance than expression from OL circuits (FIG. 6A). At low kinase levels, output expression from CL circuits was similar to OL circuits, regardless of TMP concentration. (FIG. 6B). This may be due to a lack of pOmpR-VP64 for the phosphatase regulator to dephosphorylate. However, at a given output expression level, CL circuits were substantially more robust to perturbations than OL circuits, particularly when TMP concentrations were sufficient to stabilize the phosphatase regulator (FIG. 6C).

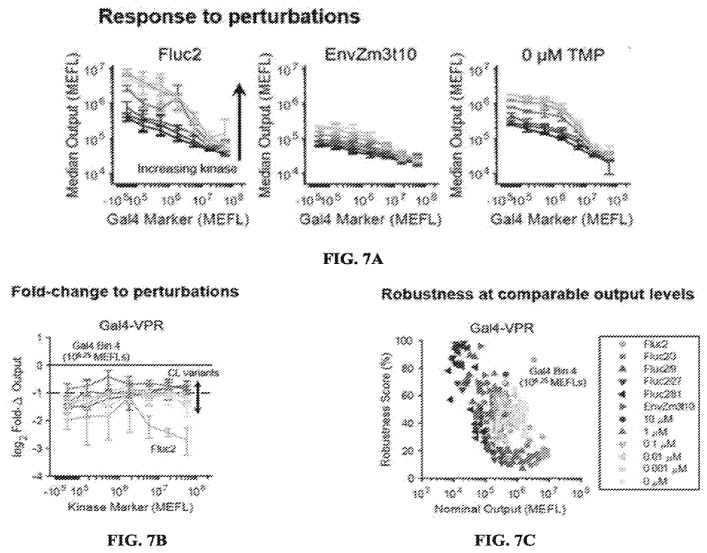

To test the ability of the feedback controller to protect output gene expression from indirect competition for transcriptional resources, HEK-293FT cells were co-transfected with CL or OL circuits, varying the dosage of kinase-encoding DNA, TMP (if CL circuits encoded a phosphatase regulator with a DDd domain), and Gal4-VPR. Gal4-VPR strongly sequesters transcriptional resources via the squelching effect, thereby reducing expression of other genes, such as the output molecule and phosphatase regulator. As with experiments using miR-FF4, CL circuits were markedly less sensitive to changes in Ga14-VPR abundance compared to OL circuits (FIG. 7A). Unlike experiments using miR-FF4, kinase abundance had little effect on the sensitivity of CL circuits to Gal4-VPR perturbation (FIG. 7B). Without being bound by theory, this difference may be explained by resource sequestration by Ga14-VPR more strongly affecting activated, rather than basal, expression. Thus, the perturbation could be weaker in cells with lower kinase abundance and thus lower activated expression, compensating for reduced feedback strength of the controller expected at lower kinase levels. Comparing robustness scores, it was again observed that CL circuits were significantly more robust to Ga14-VPR-mediated resource sequestration than comparable OL circuits (FIG. 7C).

### Cell Culture and Transfection

HEK-293FT cells were grown in DMEM (Corning) with 10% FBS (VWR). Cells were cultured to 90% confluency on the day of transfection and were plated out simultaneously with the addition of transfection reagents. Transfections were performed in 96-well pre-treated culture plates (Costar). 120 ng total DNA are pre-diluted in 10 μL Opti-MEM (Gibco) before adding the transfection reagent. In all experiments, the transfection reagent was lipofectamine 3000 at a ratio of 2 μL P3000 and 2 μL Lipo 300 per 1 μg DNA. The DNA-reagent mixtures were incubated for 10-30 minutes while cells were trypsinized and counted. After depositing the transfection mixture into appropriate wells, 40,000 HEK-293 cells suspended in 100 μL media were added.

### Flow Cytometry

Fluorescence output was measured 48 hours after transfection using an LSR-II Fortessa Flow Cytometer. To prepare samples in 96-well plates for flow cytometry, the following process was followed: Media was aspirated, 50 μL PBS (Corning) was added to wash the cells and remove FBS, the PBS was aspirated, and 40 μL Trypsin-EDTA (Corning) was added. The cells incubated for 5-10 minutes at 37° C. to allow for detachment and separation. Following incubation, 80 μL of DMEM without phenol red (Gibco) with 10% FBS was added to inactivate the trypsin. Cells were thoroughly mixed to separate and suspend individual cells. The plate(s) were then spun down at 400×g for 4 minutes, and the leftover media was aspirated. Cells were resuspended in 170 μL of PBS supplemented with 1% BSA (Thermo Fisher), 5 mM EDTA (VWR), and 0.1% sodium azide (Sigma-Aldrich) to prevent clumping. 10,000-50,000 cells were collected per sample using an HTS unit attached to the cytometer. The flow rate was set to collect 500-2000 events/second.

Data analysis can be performed, for example, using MATLAB scripts. Arbitrary fluorescence was converted to standardized MEFL units using RCP-30-5A beads (Spherotech) and the TASBE pipeline process. Fluorescence compensation was performed by subtracting autofluorescence, computing linear fits between channels in single-color transfected cells, then using the fit slopes as matrix coefficients for matrix-based signal deconvolution. Single cells were isolated by drawing morphological gates based on cellular side-scatter and forward-scatter. Transfected cells were isolated by manually gating for cells positive for the transfection marker or the output reporter. Cells were analytically binned using fluorescent markers that reported the dosage of certain genes (e.g., kinase, phosphatase, perturbation module) as indicated in the Drawings.

### CONCLUSIONS

Phosphorylation-based feedback controllers can be used to tunably and robustly control gene expression in mammalian cells. In particular, output expression from CL circuits with such controllers is robust to perturbations both from off-target regulation and resource competition that affect the transcription and translation processes of the circuit output. These results demonstrate that the controller is a general-purpose tool for building robust circuits that could have broad applications. For example, this phosphorylation-based topology could be combined with TCS-based receptors to create tunable and robust synthetic signaling architectures in mammalian cells. Such devices could be useful for driving cell fate conversions or developing sophisticated cell therapies that can sense and respond to their environment. This phosphatase and feedback architecture may also be useful in combination with a phosphorylation-based classifier to create advanced cell classifier circuits with altered input-output functions.

### Modular Plasmid Cloning Scheme

Plasmids were constructed using a modular Golden Gate strategy. Briefly, basic parts (“Level 0s” [pL0s]—insulators, promoters, 5′UTRs, coding sequences, 3′UTRs, and terminators) were created via standard cloning techniques. Typically, pL0s were generated via PCR (Q5 and OneTaq hot-start polymerases, New England BioLabs (NEB)) followed by In-Fusion (Takara Bio) or direct synthesis of shorter inserts followed by ligation into pL0 backbones. Oligonucleotides were synthesized by Integrated DNA Technologies (IDT) or SGI-DNA. pL0s were assembled into transcription units (TUs—“Level 1s” [pL1s]) using BsaI Golden Gate reactions (10-50 cycles between 16degC and 37degC, T4 DNA ligase). TUs were assembled into multi-TU plasmids (“Level 2s” [pL2s]) using SapI Golden Gate reactions. All restriction enzymes and T4 ligase were obtained from NEB. Plasmids were transformed into Stellar E. coli competent cells (Takara Bio). Transformed Stellar cells were plated on LB agar (VWR) and propagated in TB media (Sigma-Aldrich). Carbenicillin (100 μg/mL), kanamycin (50 m/mL), and/or spectinomycin (100 m/mL) were added to the plates or media in accordance with the resistance gene(s) on each plasmid. All plasmids were extracted from cells with QIAprep Spin Miniprep and QIAGEN Plasmid Plus Midiprep Kits. Plasmid sequences were verified by Sanger sequencing at Quintara Biosciences. Genbank files for each plasmid and vector backbone used in this study are provided in Supplementary Data. Plasmid sequences were created and annotated using Geneious (Biomatters).

In addition to the above, a new scheme for engineering synthetic promoters using “Level Sub-0” (pSub0) plasmids was developed. In this system, promoters are divided into up to 10 pSub0 fragments. Because the core elements of a promoter are typically at the 3′ end, the pSub0 position vectors start with the 3′-most element and move towards the 5′ of the promoter. Promoter position 1 (pP1) contains the transcription start site (TSS), the +1 position for transcription initiation, and surrounding sequences. pP1 can also optionally contain transcriptional repressor binding sites. pP2 contains the TATA box and other upstream core promoter elements as desired. Many of the pP1 and pP2 sequences were derived from the minimal promoters studied by Ede et al. Because the spacing between the TATA box and +1 site are critical, each minimal promoter was broken apart at equivalent positions such that they can be interchanged. pP1 and pP2 parts were generally created via PCR reactions using the base pSub0 backbone as a template and adding the inserts via primer overhangs and In-Fusion cloning. Positions 3-10 (pP3-10) are ‘enhancer’ positions, which generally encode binding sites (i.e. response elements) for transcriptional activators (such as the RRs in this Example), or enhancers from constitutive promoters (not done in this study). pP3-10 plasmids were made by directly ligating annealed primers into pSub0 pP3-10 backbones or through PCR followed by In-Fusion. The annealed primers were synthesized with 4 bp offsets at each end to naturally create overhangs when annealed. All pSub0 plasmids include BsaI binding sites in an analogous position to pL0s, such that pSub0s can be used directly in place of pL0s when generating pL1s (the overhangs are compatible for up to four pSub0 inserts). Because pSub0s and pL0s use BsaI for cloning in the same way, insertion into pL0 backbones using BsaI Golden Gate is inefficient. To more efficiently clone pSub0s into pL0 P.2 (level 0 promoter) plasmids, Golden Gate reaction was performed with the pSub0s separately from the pL0 backbone, then ligated the Golden Gate product with a pre-fragmented and gel-extracted pL0 backbone.

### Cell Culture

HEK-293 cells (ATCC), HEK-293FT cells (Thermo Fisher), and HeLa cells (ATCC) were maintained in Dulbecco's modified Eagle media (DMEM) containing 4.5 g/L glucose, L-glutamine, and sodium pyruvate (Corning) supplemented with 10% fetal bovine serum (FBS, from VWR). All cell lines used in the study were grown in a humidified incubator at 37deg and 5% CO₂. All cell lines tested negative for mycoplasma.

### Transfections

Cells were cultured to 90% confluency on the day of transfection, trypsinized, and added to new plates simultaneously with the addition of plasmid-transfection reagent mixtures (reverse transfection). Transfections were performed in 384-, 96-, 24-, or 6-well pre-treated tissue culture plates (Costar). Following are the volumes, number of cells, and concentrations of reagents used for 96-well transfections; for 384-, 24- and 6-well transfections, all values were scaled by a factor of 0.2, 5, or 30, respectively. 120 ng total DNA was diluted into 10 μL Opti-MEM (Gibco) and lightly vortexed. For poly-transfection experiments, the DNA dosage was subdivided equally among each complex (e.g. for two complexes, we delivered 60 ng DNA in each, 40 ng for three complexes, etc.) The transfection regent was then added and samples were lightly vortexed again. The DNA-reagent mixtures were incubated for 10-30 minutes while cells were trypsinized and counted. After depositing the transfection mixtures into appropriate wells, 40,000 HEK-293, 40,000 HEK-293FT, or 10,000 HeLa cells suspended in 100 μL media were added. Lipofectamine 3000 was used at a ratio of 2 μL P3000 and 2 μL Lipo 300 per 1 μg DNA. PEI MAX (Polysciences VWR) was used at a ratio of 3 μL PEI per 1 μg DNA. FuGENE6 (Promega) was used at a ratio of 3 μL FuGENE6 per 1 μg DNA. Viafect (Promega) was used at a ratio of 3 μL Viafect per 1 μg DNA. The media of the transfected cells was not replaced between transfection and data collection. For all transfections with TMP (Sigma-Aldrich) or 4-OHT (Sigma-Aldrich), the small molecules were added concurrently with transfection complexes. In each transfection reagent-DNA complex, an hEF1a-driven transfection marker was included to indicate the dosage of DNA delivered to each cell.

### Luciferase Assays and Analysis

To measure RR-driven luminescence output, the Promega Nano-Glo Dual-Luciferase Reporter Assay System was used, following the manufacturer's instructions. Briefly, 6,000 HEK-293FT cells were transfected using the FuGENE6 reagent with 25 ng total DNA comprising the plasmids hPGK:Fluc2 (pGL4.53), an hEF1a-driven HK, an hEF1a-driven RR, an RR-driven promoter expressing NanoLuc, and filler DNA at 5 ng each. The cells were cultured in 20 uL DMEM supplemented with 10% FBS in 384-well plates with solid white walls and bottoms (Thermo Fisher) to facilitate luminescence measurements. 48 hours post-transfection, cells were removed from the incubator and allowed to cool to room temperature. 20 μL of ONE-Glo EX Reagent was added directly to the cultures, and cells were incubated for 3 minutes on an orbital shaker at 900 revolutions per minute (RPM). Fluc2 signal was measured on a BioTek Synergy H1 hybrid reader, with an integration time of 1 s. 20 μL of NanoDLR Stop & Glo Reagent was then added, and cells were again incubated for 3 minutes on an orbital shaker at 900 RPM. After waiting an additional 10 minutes following shaking, NanoLuc signal was measured on the same BioTek plate reader, with an integration time of 1 s. NanoLuc signals were normalized by dividing by the Fluc2 signals, thereby accounting for differences in transfection efficiency among wells.

### Identification of Optimal Orthogonal TCS Pairs

To identify the optimal set of orthogonal TCS interactions, a MATLAB script was used to score all possible combinations of 4-7 HK—RR protein pairs. The script uses a scoring function to evaluate each subset of HKs and RRs. The data input into the scoring function is a matrix of output expression levels driven by the RRs in the presence of the selected HKs. The scoring function first identifies a reference value for each row and column by iteratively finding the maximum value in the matrix, blocking off the rest of the values in its row and column, then repeating until each row and column has one reference value. The reference value is then divided by the rest of the values in its row and column, and the quotients are multiplied together to give a score. The scores for each reference value are then again multiplied together to get a final score for a particular combination of HKs and RRs. After iterating through all possible such combinations, the highest final score for a given submatrix size is selected. The method gave qualitatively orthogonal combinations for up to 7 TCS pairs.

### Flow Cytometry

To prepare samples in 96-well plates for flow cytometry, the following procedure was followed: media was aspirated, 50 μL PBS (Corning) was added to wash the cells and remove FBS, the PBS was aspirated, and 40 μL Trypsin-EDTA (Corning) was added. The cells were incubated for 5-10 minutes at 37deg C. to allow for detachment and separation. Following incubation, 80 μL of DMEM without phenol red (Gibco) with 10% FBS was added to inactivate the trypsin. Cells were thoroughly mixed to separate and suspend individual cells. The plate(s) were then spun down at 400×g for 4 minutes, and the leftover media was aspirated. Cells were resuspended in 170 μL flow buffer (PBS supplemented with 1% BSA (Thermo Fisher), 5 mM EDTA (VWR), and 0.1% sodium azide (Sigma-Aldrich) to prevent clumping). For prepping plates of cells with larger surface areas, all volumes were scaled up in proportion to surface area and samples were transferred to 5 mL polystyrene FACS tubes (Falcon) after trypsinization. For standard co-transfections, 10,000-50,000 cells were collected per sample. For the poly-transfection experiment and transfections into cells harboring an existing lentiviral integration, 100,000-200,000 cells were collected per sample.

For all experiments, samples were collected on a BD LSR Fortessa equipped with a 405 nm laser with 450/50 nm filter (‘Pacific Blue’) for measuring TagBFP or EBFP2, 488 laser with 530/30 filter (‘FITC’) for measuring EYFP or mNeonGreen, 561 nm laser with 582/15 nm filter (‘PE’) or 610/20 nm filter (‘PE-Texas Red’) for measuring mKate2 or mKO2, and 640 laser with 780/60 nm filter (‘APC-Cy7’) for measuring iRFP720. 500-2000 events/s were collected either in tubes via the collection port or in 96-well plates via the high-throughput sampler (HTS). All events were recorded and compensation was not applied until processing the data (see below).

Analysis of flow cytometry data was performed using a MATLAB-based flow cytometry analysis pipeline. Basic processing steps follow the procedures described previously. In addition, a new poly-transfection technique and associated methods were used to characterize and optimize feedback controller circuits. Poly-transfection enables rapid and accurate assessment of dose-response curves for genetic components, such as the kinases and phosphatases in the circuits described herein.

Multi-dimensional binning of poly-transfection data was performed by first defining bin edges in each dimension (i.e. for the transfection markers for each poly-transfection complex), then assigning each cell to a bin where the cell's expression of these markers was less-than-or-equal-to the high bin edges and greater-than the low bin edges. Bins with three or fewer cells were ignored (values set to NaN in the MATLAB code) to avoid skewing by outliers in sparsely-populated samples (e.g. HeLa cells). In order to avoid the artefact of negative fold-changes, non-positive fluorescence values were discarded prior to making measurements on binned or gated populations. In the second and third experimental repeats of the miRNA-dependent signaling/classifier data in, a newly prepared Output Marker plasmid was later discovered to have −8-fold lower concentration than expected due to a measurement error on the nanodrop. To account for this, the bins for the Output Marker in those samples are shifted down by 10×(so as to match the same bin boundaries as in the first repeat).

To find the optimal ratio of components in the miR-21 sensor for high cell classification accuracy, ratios between 1000:1 to 1:1000 of K:P and output plasmid:K/P were scanned, roughly halving the ratio between steps. At each combination of ratios, a trajectory was computed and all cells within 0.25 biexponential units of the trajectory based on Euclidean distance were recorded. Accuracy was computed as described below, and accuracy values were compared across all ratios for each experimental repeat. From this scanning of trajectories at different ratios of components, it was determined that a 1:1:0.5 ratio of K:P:Output plasmid gave the highest accuracy.

In the case of simple co-transfections and sub-sampled trajectories, cells were considered to be transfected if they were positive for the output/transfection marker or the output reporter. When computing summary statistics from binned data, such thresholding is unnecessary since binning already isolates the cell sub-population for measurement.

Calculation of cell classification metrics Sensitivity was defined as the percent of cells positive for the output reporter in HeLa cells transfected with the T21 circuit variant. Specificity was defined as 100 minus the percent of cells positive for the output in HeLa cells with the TFF4 variant or in HEK-293 cells with the T21 variant. The former was considered the more ideal comparison for evaluating classification performance due to higher overall expression of the circuit in HeLa cells compared to HEKs. Accuracy was computed by averaging sensitivity and specificity.

### Calculation of Fold-Changes and Robustness Scores

For quantifying the effects of EnvZ variants and perturbations, fold-changes were calculated by dividing the median output level of each sample by that of the equivalent sample in the absence of the EnvZ variant or perturbation. For perturbation experiments, the level of output absent perturbation is referred to as the nominal output level.

$$Fold - Δ ( Input / perturbation binx ) = \frac{Output ( Input / perturbation binx )}{Output ( Input / perturbation bin 1 )}$$

Where log₂-transformed fold-changes are shown for experiments with multiple repeats, the values shown are the mean of the log₂-transformed fold-changes, rather than the log₂-transformation of the mean of the fold-changes. This order of operations ensures that standard deviations of the fold-changes can be computed directly on the log₂-transformed scale.

Robustness scores were calculated from the fold-changes using the formula below:

Robustness(Perturbation binx)=100·(1−|1−Fold-Δ(Perturbation binx|)

### Quantification of Cell-to-Cell Output Variance

To measure noise, the interquartile range (IQR) of the output distributions was calculated. Because the median was chosen to represent the middle of the distribution, the IQR is a corresponding non-parametric measurement of noise. Since gene expression noise is approximately log-distributed, data were log₁₀-transformed prior to computing the IQR. As with calculations of the medians, negative fluorescent values were discarded when computing the IQR to avoid artefacts.

### Model Fitting

Where possible, fluorescent reporters were used to estimate the concentration of a molecular species for the purpose of model fitting.

For fitting all models, the MATLAB function ‘lsqcurvefit( )’ was used, which minimizes the sum of the squares of the residuals between the model and the data. In general, fits were made with cells subsampled from bins, as indicated for each figure. Fits were always performed individually per experimental repeat, then means and standard deviations were computed for individual fit parameters.

Goodness of fit was measured by computing the normalized root-mean-square error CV(RMSE) using the following formula:

$$CV ( RMSE ) = \frac{\sqrt{\frac{1}{y} {{Σ}_{i} ( y ( {x}_{i} ) - f ( {x}_{i} ) )}^{2}}}{\overline{y}}$$

where y(xᵢ) is the value of the data at the input value xᵢ, y̅ is the mean of y for all values of x, and f(xᵢ) is the function output at input value xᵢ.

### Fitting Functions

Activation of transcription by OmpR-VP64:

$$y = {α}_{0} + ( α - {α}_{0} ) \frac{{x}^{2}}{{K}_{1 / 2}^{2} + {x}^{2}}$$

The cooperativity of OmpR was assumed to be two because it forms a dimer once phosphorylated to bind DNA.

Activation of OmpR-VP64-driven expression by kinase:

$$y = {α}_{0} + ( α - {α}_{0} ) \frac{{x}^{2}}{{K}_{1 / 2}^{2} + {x}^{2}}$$

Deactivation of OmpR-VP64 by phosphatase:

$$y = {α}_{0} + ( α - {α}_{0} ) \frac{{K}_{1 / 2}^{2}}{{K}_{1 / 2}^{2} + {x}^{2}}$$

While OmpR-VP64 has not been completely tuned over to P-OmpR-VP64, the amount of P-OmpR-VP64 is assumed to be proportional to the level of kinase because the production rate is only dependent on the kinase. In the presence of the phosphatase, the decay rate becomes overwritten by the dephosphorylation reaction. Thus, these protein abundances can be plugged directly into the OmpR-VP64 activation function, such that the kinase is proportional to OmpR and the phosphatase is inversely so. Because the of the inversion, the phosphatase function becomes a repression-form Hill function.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A feedback controller circuit comprising:
(i) an input circuit comprising:
(a) a constitutive promoter operably linked to a nucleotide sequence encoding an activator; and
(b) a constitutive promoter operably linked to a nucleotide sequence encoding a kinase that phosphorylates the activator and produces a phosphorylated activator;
(ii) a tuning circuit comprising a promoter operably linked to a nucleotide sequence encoding a phosphatase regulator comprising a phosphatase domain and a degradation domain, wherein the phosphatase domain dephosphorylates the phosphorylated activator,
wherein the phosphatase domain comprises a mutation in a catalytic and ATP-binding domain and/or is truncated at its N-terminus relative to a phosphatase comprising the amino acid sequence of SEQ ID NO: 1; and
(iii) a signal circuit comprising an activatable promoter operably linked to a nucleotide sequence encoding an output molecule, wherein the activatable promoter is activatable by the phosphorylated activator.

2. The feedback controller circuit of claim 1, wherein the kinase, the phosphatase, and/or the activator are members of a bacterial two-component signaling system.

3. The feedback controller circuit of claim 2, wherein bacterial two-component system comprises a histidine kinase comprising an amino acid sequence motif of HEXXN, HEXXT, or HDXXXP, wherein X is any amino acid, and a response regulator.

4. The feedback controller circuit of claim 3, wherein the phosphatase is a histidine kinase variant comprising an amino acid substitution in the E or D of the HEXXN, HEXXT, or HDXXXP motif.

5. The feedback controller circuit of claim 4, wherein the phosphatase comprises an alanine substitution in the E or D of the HEXXN, HEXXT, or HDXXXP motif.

6. The feedback controller circuit of any one of claims 1-5, wherein the phosphatase comprises an amino acid substitution corresponding to a D244A substitution in SEQ ID NO: 1.

7. The feedback controller circuit of any one of claims 1-6, wherein the phosphatase comprises the amino acid sequence of SEQ ID NO: 2.

8. The feedback controller circuit of any one of claims 1-5, wherein the phosphatase comprises a dimerization and histidine phosphorylation (DHp) domain of EnvZ.

9. The feedback controller circuit of claim 8, wherein the phosphatase comprises the amino acid sequence of SEQ ID NO: 4.

10. The feedback controller circuit of any one of claims 1-5, wherein the phosphatase domain is a truncated EnvZ (EnvZt) phosphatase domain.

11. The feedback controller circuit of claim 10, wherein the phosphatase domain of the phosphatase regulator lacks a signaling domain and/or a HAMP domain of EnvZ.

12. The feedback controller circuit of claim 10 or 11, wherein the truncated EnvZ phosphatase domain comprises no more than 229, 230, 231, 232, 233, 234, 235, 236, 237, or 238 amino acids corresponding to the C-terminus of the amino acid sequence of SEQ ID NO: 6.

13. The feedback controller circuit of any one of claims 1-12, wherein the phosphatase domain comprises a mutation in a catalytic and ATP-binding domain, wherein the binding affinity for ATP of a phosphatase comprising the mutation is at least 1.5, 2.0, 2.5, 3.0, 3.5, 4.0, 4.5, 5.0, 6.0, 7.0, 8.0, 9.0, or 10.0 times lower than binding affinity for ATP of a phosphatase comprising the amino acid sequence of SEQ ID NO: 1.

14. The feedback controller circuit of any one of claims 1-13, wherein the phosphatase domain comprises a substitution at an amino acid corresponding to N343 of SEQ ID NO: 1.

15. The feedback controller circuit of claim 14, wherein the phosphatase domain comprises a lysine substitution at a position corresponding to N343 of SEQ ID NO: 1.

16. The feedback controller circuit of any one of claims 1-15, wherein the phosphatase regulator further comprises a leucine zipper domain that is covalently linked to the phosphatase domain.

17. The feedback controller circuit of claim 16, wherein the leucine zipper is a GCN4 domain.

18. The feedback controller circuit of claim 17, wherein the GCN4 domain comprises the amino acid sequence of SEQ ID NO: 30.

19. The feedback controller circuit of claim 17 or 18, wherein the phosphatase regulator further comprises a degradation domain that is linked to the phosphatase domain or the leucine zipper domain.

20. The feedback controller circuit of claim 19, wherein the degradation domain is selected from the group consisting of PEST, DDd, DDe, and DDf.

21. The feedback controller circuit of claim 20, wherein the degradation domain is DDd.

22. The feedback controller circuit of any one of claims 1-21, wherein the phosphatase regulator comprises a plurality of monomers, wherein each monomer is a fusion protein comprising a phosphatase domain, a leucine zipper domain fused to the phosphatase domain, and one or more degradation domains fused to the phosphatase domain and/or leucine zipper domain.

23. The feedback controller circuit of claim 22, wherein each degradation domain is selected from the group consisting of PEST, DDd, DDe, and DDf.

24. The feedback controller circuit of claim 23, wherein each degradation domain is DDd.

25. The feedback controller circuit of any one of claims 21-24, wherein the leucine zipper is a GCN4 domain.

26. The feedback controller circuit of claim 25, wherein the GCN4 domain comprises the amino acid sequence of SEQ ID NO: 30.

27. The feedback controller circuit of any one of claims 21-26, wherein each monomer comprises an amino acid sequence with at least 80% sequence identity to the amino acid sequence of SEQ ID NO: 18.

28. The feedback controller circuit of claim 27, wherein each monomer comprises the amino acid sequence of SEQ ID NO: 18.

29. The feedback controller circuit of any one of claims 1-28, wherein the phosphatase regulator comprises an amino acid sequence with at least 80% sequence identity to the amino acid sequence of SEQ ID NO: 18.

30. The feedback controller circuit of claim 29, wherein the phosphatase regulator comprises the amino acid sequence of SEQ ID NO: 18.

31. The feedback controller circuit of any one of claims 1-30, wherein the kinase is a variant of the histidine kinase an amino acid substitution in the N, T, or P of the HEXXN, HEXXT or HDXXXP motif.

32. The feedback controller circuit of claim 31, wherein the kinase comprises an alanine substitution in the N, T, or P of the HEXXN, HEXXT, or HDXXXP motif.

33. The feedback controller circuit of any one of claims 1-32, wherein the histidine kinase is selected from the group consisting of: EnvZ, NarX, and PhoR.

34. The feedback controller circuit of any one of claims 1-33, wherein the histidine kinase is an EnvZ or portion thereof.

35. The feedback controller circuit of any one of claims 1-34, wherein the histidine kinase comprises the amino acid sequence of SEQ ID NO: 1.

36. The feedback controller circuit of any one of claims 1-35, wherein the kinase comprises an amino acid substitution corresponding to a T247A substitution in SEQ ID NO: 1.

37. The feedback controller circuit of any one of claims 1-36, wherein the kinase comprises the amino acid sequence of SEQ ID NO: 3.

38. The feedback controller circuit of any one of claims 1-34, wherein the kinase comprises two DHp domains fused to a cytoplasmic domain of EnvZ.

39. The feedback controller circuit of any one of claims 1-38, wherein the kinase comprises the amino acid sequence of SEQ ID NO: 5.

40. The feedback controller circuit of any one of claims 1-39, wherein the activator comprises a response regulator of the bacterial two-component system.

41. The feedback controller circuit of any one of claims 1-39, wherein the activator comprises a response regulator of the bacterial two-component system fused to an activation domain.

42. The feedback controller circuit of claim 41, wherein the activation domain is selected from the group consisting of VP16, VP64, p65, and VPR.

43. The feedback controller circuit of claim 42, wherein the activation domain is VP64.

44. The feedback controller circuit of any one of claims 40-43, wherein the response regulator is selected from the group consisting of OmpR, NarL, NtrC, and PhoB.

45. The feedback controller circuit of claim 44, wherein the response regulator is OmpR.

46. The feedback controller circuit of any one of claims 41-45, wherein the activator comprises VP64 and OmpR.

47. The feedback controller circuit of claim 46, wherein the activator comprises an amino acid sequence with at least 80% sequence identity to the amino acid sequence of SEQ ID NO: 69.

48. The feedback controller circuit of any one of claims 1-45, wherein the activatable promoter comprises one or more response elements that are capable of being bound by the activator.

49. The feedback controller circuit of claim 48, wherein the response element comprises one or more operators of the activator.

50. The feedback controller circuit of claim 48 or 49, wherein the activatable promoter further comprises a minimal promoter fused to the one or more response elements.

51. The feedback controller circuit of any one of claims 1-50, wherein the promoter of (ii) is the same as the activatable promoter of (iii).

52. The feedback controller circuit of claim 51, wherein the second sensor circuit and the signal circuit are comprised on the same nucleic acid, wherein the activatable promoter of (iii) controls transcription of the output molecule and the phosphatase regulator.

53. The feedback controller circuit of any one of claims 1-51, wherein the promoter of (ii) is a constitutive promoter.

54. The feedback controller of claim 53, wherein the tuning circuit and the signal circuit are comprised on different nucleic acids.

55. A cell state classifier comprising the feedback controller circuit of any one of claims 1-54, wherein the input circuit and the signal circuit each comprise one or more target sites for a first miRNA.

56. The cell state classifier of claim 55, wherein the one or more target sites for the first microRNA is located upstream and/or downstream of the nucleotide sequence encoding the activator and the nucleotide sequence encoding the kinase in the input circuit.

57. The cell state classifier of claim 55 or 56, wherein 4 target sites for the first microRNA are located upstream and/or downstream of the nucleotide sequence encoding the activator and the nucleotide sequence encoding the kinase in the input circuit.

58. The cell state classifier of any one of claims 55-57, wherein the one or more target sites for the first microRNA is located upstream and/or downstream of the nucleotide sequence encoding the output molecule in the signal circuit.

59. The cell state classifier of any one of claims 55-58, wherein 4 target sites for the first microRNA are located upstream and/or downstream of the nucleotide sequence encoding the output molecule in the signal circuit.

60. The cell state classifier of any one of claims 1-59, wherein the tuning circuit comprises one or more target sequences for a second miRNA.

61. The cell state classifier of claim 60, wherein the one or more target sites for the second microRNA is located upstream and/or downstream of the nucleotide sequence encoding the phosphatase in the tuning circuit.

62. The cell state classifier of claim 60 or 61, wherein 4 target sites for the second microRNA are located upstream and/or downstream of the nucleotide sequence encoding the phosphatase in the tuning circuit.

63. The feedback controller circuit of any one of claims 1-54 or cell state classifier of any one of claims 55-62, wherein the output molecule is a detectable molecule.

64. The feedback controller circuit of any one of claims 1-54 or cell state classifier of any one of claims 55-62, wherein the output molecule is a therapeutic molecule.

65. A cell comprising the feedback controller circuit of any one of claims 1-54 or the cell state classifier of any one of claims 55-62.

66. The cell of claim 65, wherein the cell is a prokaryotic cell.

67. The cell of claim 66, wherein the cell is a bacterial cell.

68. The cell of claim 65, wherein the cell is a eukaryotic cell.

69. The cell of claim 68, wherein the eukaryotic cell is a plant cell, insect cell, fish cell, amphibian cell, reptilian cell, avian cell, or mammalian cell.

70. The cell of claim 69, wherein the mammalian cell is a human cell.

71. The cell of any one of claims 65-70, wherein the cell is a diseased cell.

72. The cell of any one of claims 65-70, wherein the cell is a cancer cell.

73. The cell of any one of claims 65-72, wherein the cell does not express the first miRNA.

74. The cell of any one of claims 65-73, wherein the cell expresses the second miRNA.

75. The cell of any one of claim 65-72 or 74, wherein the cell expresses the first miRNA and the second miRNA.

76. The cell of any one of claims 65-73, wherein the cell does not express the first miRNA and does not express the second miRNA.

77. A method comprising maintaining the cell of any one of claims 65-76.

78. The method of claim 77, further comprising detecting the output molecule.

79. The method of claim 78, further comprising classifying the cell based on expression of the output molecule.

80. A method comprising delivering the feedback controller circuit or cell state classifier of any one of claims 1-64 to a cell and detecting the output molecule.

81. A method of treating a disease or disorder, the method comprising delivering to a cell the feedback controller circuit or cell state classifier of any one of claims 1-64, wherein the output molecule is a therapeutic molecule that is effective for treating the disease or disorder.

82. The method of claim 80 or 81, wherein the cell is a diseased cell.

83. The method of any one of claims 80-82, wherein the cell is a cancer cell.

84. A method of determining the disease or disorder state of a cell, the method comprising delivering to the cell the feedback controller circuit or cell state classifier of any one of claims 1-64.

85. The method of claim 84, wherein the cell is a diseased cell.

86. The method of claim 84 or 85, wherein the cell is a cancer cell.

87. The method of any one of claims 84-86, further comprising detecting the output molecule.

88. The method of claim 87, wherein detecting the output molecule in the cell indicates the disease or disorder.

89. The method of claim 87, wherein lack of detection of the output molecule in the cell indicates the disease or disorder.

90. A composition comprising the feedback controller circuit or cell state classifier of any one of claims 1-64.

91. A pharmaceutical composition comprising the composition of claim 90, and a pharmaceutically acceptable excipient.

92. A method of treating a disease or disorder, the method comprising administering an effective amount of the composition of claim 90 or 91 to a subject in need thereof, wherein the output molecule is a therapeutic molecule that is effective for treating the disease or disorder.

93. A method of diagnosing a disease or disorder comprising administering an effective amount of the composition of claim 90 or 91 to a subject in need thereof, and detecting the output molecule.
